(19) United States
(12) Patent Application Publication
(10) Pub. No.: US 2005/0009849 A1
(43) Pub. Date: Jan. 13, 2005

(54) Pyridopyrimidine kinase inhibitors

(76) Inventors: Darren R. Veach, New York, NY (US); William Bornmann, New York, NY (US); Bayard D. Clarkson, New York, NY (US); Nikolas von Bubonoff, Munich (DE); Justus Duyster, Munich (DE)

Correspondence Address: Choate, Hall & Stewart, Exchange Place, 53 State Street, Boston, MA, 02109, US

(21) Appl. No.: 10/751,703

(22) Filed: Jan. 5, 2004

Related U.S. Application Data
(60) Provisional application No. 60/437,936, filed on Jan. 3, 2003.
(60) Provisional application No. 60/500,978, filed on Sep. 8, 2003.

(51) Int. Cl. A61K 31/519; C07D 487/02

(52) U.S. Cl. 514/264.11; 544/280

(57) ABSTRACT

The present invention provides compounds of formula (0): 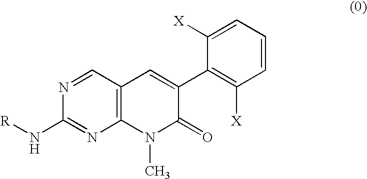 as described generally and in classes and subclasses herein. The present invention additionally provides pharmaceutical compositions comprising compounds of formula (0) and provides methods of treating cancer comprising administering a compound of formula (0).

### PRIORITY INFORMATION

The present application claims priority under 35 U.S.C. § 119(e) to co-pending provisional applications U.S. Ser. No. 60/437,936, filed Jan. 3, 2003, entitled “Pyridopyrimidine Kinase Inhibitors”, and U.S. Ser. No. 60/500,978, filed Sep. 8, 2003; the entire contents of each of which are incorporated herein by reference. The subject matter in the present application is also related to the subject matter in provisional application 60/376,520, filed Apr. 30, 2002, entitled “Crystal Structures of the Kinase Domain of c-Abl complexed to Small Molecule Inhibitors and Design of Better c-Abl Inhibitors,” now expired, the entire contents of which are incorporated herein by reference.

### BACKGROUND OF THE INVENTION

Tyrosine kinases are a family of tightly regulated enzymes, and the aberrant activation of various members of this family is one of the hallmarks of cancer. In chronic myelogenous leukemia (CML), the Abelson tyrosine kinase is improperly activated by the accidental fusion of the bcr gene with the gene encoding the intracellular non-receptor tyrosine kinase, c-Abl. Wild-type c-Abl is a large (˜1150 residue) protein. The N-terminal half (˜530 residues) of c-Abl bears 42% sequence identity to the Src family of tyrosine kinases (excluding the N-terminal unique domain) and shares a similar domain organization, containing two modular peptide binding units (the SH2 and SH3 domains) followed by a tyrosine kinase domain. However, c-Abl is distinct from the Src kinases in that it lacks a critical tyrosine residue that follows the kinase domain of c-Src. Phosphorylation of this tyrosine residue results in the inactivation of the Src kinases. The C-terminal half of c-Abl contains DNA and actin-binding domains interspersed with sites of phosphorylation and other short recognition motifs, including proline-rich segments and nuclear localization signals (Van Etten, Trends Cell Biol. 9:179-186, 1999, incorporated herein by reference). Under normal conditions, c-Abl exists in a regulated state with very low kinase activity (Jackson et al., Embo. J 8:449-456, 1989, incorporated herein by reference). In CML, however, the fusion of Bcr to the N-terminus of c-Abl results in the constitutive activation of Abl kinase activity, by a mechanism that is not well understood. Bcr-Abl phosphorylates cellular proteins extensively and transforms cells, making them growth factor independent (Druker et al. Mol. Cell. Biol. 13:1728-1736, 1993; incorporated herein by reference).

The catalytic domains of eukaryotic Ser/Thr and tyrosine kinases are highly conserved in sequence and structure. The kinase domain has a bilobal structure. The N-terminal lobe (N-lobe) contains a β-sheet and one conserved α-helix (helix C). The C-terminal lobe (C-lobe) is largely helical. At the interface between the two lobes, a number of highly conserved residues form the ATP-binding pocket and the catalytic machinery. Small molecule inhibitors of protein kinases that have been discovered to date appear to invariably bind to the kinase domain at this interfacial cleft between the two lobes, displacing ATP.

Of importance for the development of treatments for CML are a number of small molecule compounds that can inhibit the tyrosine kinase activity of the Bcr-Abl protein with some degree of selectivity. A member of the class of 2-phenylaminopyrimidine compounds, STI-571 (originally called CGP57148B and now known as Gleevec, FIG. 1A), was identified in 1996 by Novartis and shows promise as a therapeutic agent (Druker et al. Nat. Med 2:561-66, 1996; incorporated herein by reference). STI-571 has now been approved by the FDA for treatment of CML.

The crystal structure of the kinase domain of Abl complexed to a variant of STI-571 (AblK:STI-571 (variant)), lacking the piperazinyl group has been reported (FIG. 1A) (Schindler et al. Science 289:1938-1942, 2000; incorporated herein by reference). The structure showed that STI-571 recognizes an inactive and unphosphorylated conformation of Abl specifically. The inactive conformation of the Abl kinase domain appears to be crucial to the selectivity of STI-571, since this conformation differs from the inactive conformations of other tyrosine kinases, such as the closely related Src kinases, against which STI-571 is inactive. The potency of STI-571 against the activated forms of Bcr-Abl presumably arises from the dynamic nature of kinase molecules, which can switch between the inactive and active forms transiently, allowing STI-571 to gain entry.

It has been shown recently that a class of inhibitors based on the pyrido-[2,3-d]pyrimidine core compounds are also active against Abl (Dorsey et al. Cancer Res. 60:3127-3131, 2000; incorporated herein by reference). A closely related compound (PD173955, Parke-Davis) was shown to block cell proliferation and mitotic progression through inhibition of the Src family of tyrosine kinases (Moasser et al. Cancer Res. 59:6145-6152, 1999; incorporated herein by reference). In subsequent studies we have shown that PD173955 is effective at shutting down the kinase activity of Bcr-Abl with an IC₅₀ of 1-2 nM in vitro. In vivo studies of PD173955 on Bcr-Abl containing cell lines also reveal it to be a highly effective inhibitor, with IC₅₀ ranging from 2 to 35 nM on CML cell lines (Swendeman et al., accompanying paper). Like STI-571, PD173955 is also a potent inhibitor of Kit-ligand dependent cell proliferation through inhibition of the receptor tyrosine kinase c-Kit (IC₅₀˜50 nM). Comparisons with STI-571 reveal that PD173955 is even more effective at shutting down the kinase activity of c-Abl. In contrast to STI-571, PD173955 is also a potent inhibitor of Src kinases (Moasser et al. Cancer Res. 59:6145-6152, 1999; incorporated herein by reference).

There remains a need for other compounds such as pyrido-[2,3-d]pyrimidine derivatives which perform superior to the known inhibitors of Bcr-Abl with fewer side effects and the inhibitory activity is maintained against clinically relevant mutant forms of BCR-ABL.

### DESCRIPTION OF THE DRAWINGS

FIG. 1 shows chemical structures of A, STI-571 and B, PD173955. The core compounds from which these two inhibitors were developed are shown in bold lines. In A, a red box outlines the STI-571 variant and a red circle denotes the position where a carbon atom is replaced by a nitrogen atom in the variant.

FIG. 2 shows conformational changes in the activation loop upon activation of protein kinases. On the left is shown three tyrosine kinases (Hck, Irk and Abl) in their inactive states showing distinct conformations of the activation loop. On the right is shown the crystal structure of Lck, which illustrates the conformation of the activation loop that all kinases bear upon activation by phosphorylation.

FIG. 3 is a ribbon representation of the structure of the Abl kinase domain (green) in complex with A, STI-571 (stick model) and B, PD173955 (stick model). The activation loops and the van der Waals surfaces corresponding to the inhibitors are colored blue and red for STI-571 and PD173955, respectively. The DFG motif situated at the N-terminus of the activation loop is colored gold. Helix αC and the inter-lobe connector are colored dark green.

FIG. 4 is a schematic diagram of the interactions made by A, STI-571 and B, PD173955 with Abl. Protein residues are labeled and shown in stick representation. Nitrogen atoms are colored blue, oxygen atoms are colored red, chlorine atoms are colored green, sulphur atoms are colored yellow, protein carbon atoms are colored brown, and inhibitor carbon atoms are colored green and magenta for STI-571 and PD173955, respectively. Hydrogen bonds are indicated with dotted lines along with their distances and residues making van der Waal interactions with the inhibitor are circled with dotted lines. Additional water-mediated interactions have been omitted.

FIG. 5. A, Comparison of STI-571 variant (red) with STI-571 (green) binding to Abl. Residues whose sidechains are shown and labeled are those that make additional van der Waal interactions with the piperazinyl ring of STI-571. Also shown are the two hydrogen bonds (dotted lines) made by the carbonyl oxygen atoms (red) of Ile 360 and His 361 with the piperazinyl ring. Parts of the protein have been cut away for clarity. B, Comparison of STI-571 (green) and PD173955 (magenta) binding to Abl.

FIG. 6 shows a comparison of the activation loops of A, Abl kinase bound to STI-571 (green), B, Abl kinase bound to PD173955 (red), and C, Active Lck (magenta). Dotted lines indicate salt-bridges between the phosphorylated Tyr 394 and Arg 387 in Lck.

FIG. 7. A, PD173955 shown as a van der Waals surface (red) modeled into the Abl kinase domain (green) conformation that binds to STI-571. No major clashes occur with the activation loop (blue), or any other parts of the protein. B, van der Waals representation of STI-571 (blue) in the conformation of Abl kinase (green) that crystallized with PD173955. In this case, major clashes occurs with STI-571 and the activation loop (red). The P-loop is shown in dark green in both A and B.

FIG. 8 shows results from activity assays of Abl kinase in the presence of A, STI-571 and B, PD173955. The dose response of phosphorylated Abl (pAbl) is shown in blue and that of dephosphorylated Abl is shown in pink. The reaction rates are corrected for the rate of a control reaction in the absence of Abl kinase. The amount of Src used did not give a significant signal in the spectrophotometric assay.

FIG. 9 shows the chemical structures of analogues of PD173955.

FIG. 10 is a comparison of the inhibitory activities of PD16, 17, and other analogs; PD16 is the most potent pyridopyrimidine we have yet studied for Bcr-Abl expressing cells (eg R10 Negative subclone of M07e/p210^{bcr-abl}).

FIGS. 11 and 12. PD16 is about 4-fold more inhibitory than PD17 to highly enriched CD34+GM progenitors from a patient in chronic phase as well as to blast cells from a patient in blastic phase. In these examples, the blastic phase cells were more sensitive to both PD16 and 17 (and ST1571) than the chronic phase cells, but the inhibitory concentrations vary among patients and one must always test different drugs vs the same cells under identical conditions for valid comparison. FIG. 12 shows blast cells from the same patient exposed to inhibitors with and without G-CSF and GM-CSF. Like KL, these cytokines have some protective effect vs the inhibitors and the IC₅₀ is 3-5× higher with all 3 drugs than in the absence of cytokines.

FIGS. 13 and 14 show the relative sensitivity of various human tumor cell lines to PD173955 and to PD173955 and STI 571.

FIG. 15 is a model showing that PD16 probably forms an additional hydrogen bond that may account for its tighter binding and ˜4-fold greater inhibition. Unlike ST1571 which forms 11 hydrogen bonds and 21 van der Waals interactions when bound to (inactive) abl kinase, PD17 only forms 2 hydrogen bonds and has only 11 van der Waals interactions due to the substitution of a hydroxymethyl group at position 3 on the phenyl ring in PD16 for the methylthioether in PD17.

FIG. 16 shows representative experiments comparing the effects of PD17 on highly enriched normal and CML CD34+ cells stimulated with appropriate cytokines to induce them to differentiate and grow mainly along the granulocyte/monocyte or erythroid lineages. In many experiments the CD34+ blasts were further depleted of GM, megakaryocyte or lymphocyte progenitors (CD 13, 14, 15, 46, 61, 19-) or of erythroid and megakaryocyte progenitors (CD36-, Glycophorin-, CD41/61-) in order to study the drug's effects on purer erythroid or GM progenitors.

FIG. 17 shows data from proliferative assays using R10-cells. From these assays, the concentration of each compound needed to inhibit growth by 50% or by 95-99% is shown.

FIG. 18 shows the chemical structures of inventive compounds. All compounds are based on a central bicyclic pyrido-[2,3-d]pyrimidine with a dichlorophenyl substituent on the right (FIG. 18A). R denotes different substituents bound to the pyrimidine ring at position 2 (FIG. 18B). All substituents were based on a phenylamine, itself carrying various substituents either at position 3 or 4.

FIG. 19 shows the effect of PD166326 on Ba/F3 cells. PD166326 are potent and specific inhibitors of a Bcr-Abl dependent phenotype in Ba/F3 cells expressing wild-type, activation loop, and P loop mutant forms of BCR-ABL, but not BCR-ABL/T3151. Ba/F3 cells were transformed with wild-type and mutant forms of BCR-ABL. Parental and Bcr-Abl dependent Ba/F cells were incubated for 24 and 48 hours without and in the presence of inhibitor at the indicated concentrations. Proliferation was measured in an MTS tetrazolium-based assay by absorbance of formazam at 490 nm. Two independent experiments per compound were performed. Values are expressed as mean ± SEM of triplicates. Representative results of one experiment after 48 hours of incubation with PD166326.

FIG. 20 shows the effect of SKI DV-MO 16 on Ba/F3 cells. SKI DV-MO 16 are potent and specific inhibitors of a Bcr-Abl dependent phenotype in Ba/F3 cells expressing wild-type, activation loop, and P loop mutant forms of BCR-ABL, but not BCR-ABL/T3151. Ba/F3 cells were transformed with wild-type and mutant forms of BCR-ABL. Parental and Bcr-Abl dependent Ba/F cells were incubated for 24 and 48 hours without and in the presence of inhibitor at the indicated concentrations. Proliferation was measured in an MTS tetrazolium-based assay by absorbance of formazam at 490 nm. Two independent experiments per compound were performed. Values are expressed as mean ± SEM of triplicates. Representative results of one experiment after 48 hours of incubation with SKI DV-M016 (FIG. 3) are shown.

FIG. 21 shows the effect of PD180970 on Ba/F3 cells. PD180970 is inferior to the most inhibitory compounds in terms of potency and range of specific inhibition of a Bcr-Abl dependent phenotype. Cellular proliferation has been measured as described in the legend for FIGS. 19 and 20. PD180970 requires five-fold higher concentrations for inhibition and causes unspecific inhibition of parental Ba/F3 cells at concentrations that are required for complete inhibition of P loop mutants.

FIG. 22 is a graphical representation of wild-type and mutant Bcr-Abl cellular sensitivities towards pyrido-pyrimidine-compounds. Shown are concentrations where 50 (FIG. 22A, IC50) or 95 percent (FIG. 22B, IC₉₅) of growth inhibition occurred after 48 hours of culture as determined by MTS tetrazolium-based proliferation assays. The compounds are ordered from left to right by the extent of inhibition of Ba/F3 cells transformed by wild-type BCR-ABL, and from front to rear by the extent of inhibition exerted on Ba/F3 cells expressing wild-type and different mutant forms of BCR-ABL.

FIG. 23 shows the fold increases in cellular IC₅₀ and IC₉₅ values for mutant forms of Bcr-Abl relative to wild-type. FIG. 23A. Shown is the mean relative activity of 13 pyrido-pyrimidines towards the activation loop mutant H396P and the P loop mutants E255K, Y253H, and E255V in relation to wild-type cellular IC50 and IC95 values. Thus, the factor for the activation loop mutant H396P is 1, representing equal sensitivity. The fold increase for E255K, Y253H, and E255V mirrors the lower sensitivity of P loop mutants. FIG. 23B. The mean fold increase is lower in a subgroup of five inhibitors, SKI DV-M016, SKI DV 2-87, PD173958, PD173956, and SKI DV 1-10-biotinyl. FIG. 23C. Relative activity of the subgroup of pyrido-pyrimidines shown in FIG. 6B (light grey) as compared to imatinib (dark grey). The IC₅₀ value for cellular inhibition of H396P by imatinib in Ba/F3 cells was 2.5 μM, compared to 0.2 μM in Ba/F3 cells expressing wild-type Bcr-Abl (Factor: 10) (von Bubnoff, N., Schneller, F., Peschel, C., and Duyster, J. BCR-ABL gene mutations in relation to clinical resistance of Philadelphia-chromosome-positive leukaemia to ST1571: a prospective study. Lancet, 359: 487-491, 2002; incorporated herein by reference). IC₅₀ values for imatinib in P loop mutants was not reached even after increasing the imatinib concentration to 10 μM. Therefore, respective factors exceed 40.

FIG. 24 demostrates that growth suppression is accompanied by inhibition of Bcr-Abl autophosphorylation. Ba/F3 cells expressing wild-type and mutant Bcr-Abl were incubated without and in the presence of PD166326 (FIG. 24A), SKI DV-M016 (FIG. 24B), and PD180970 (FIG. 24C) at the indicated concentrations. Whole cell lysates were subjected to SDS-PAGE. Blots were probed with antibodies to phosphotyrosine (left panel) and Abl (right panel).

FIG. 25 shows that inhibition of Bcr-Abl autophosphorylation and suppression of growth is followed by induction of apoptosis in a Bcr-Abl-dependent phenotype. Parental Ba/F cells (FIG. 25G) and Bcr-Abl transformed Ba/F3 cells (FIG. 25A: Bcr-Abl wild-type; FIG. 8B: Bcr-Abl H396P; FIG. 25C: Bcr-Abl E255K; FIG. 25D: Bcr-Abl Y253H; FIG. 25E: Bcr-Abl E255V; FIG. 25F: Bcr-Abl T3151) were measured for induction of apoptosis when cultured in the presence of PD166326 at different concentrations. Annexin V-positive cells at indicated time points are depicted. Two experiments per cell line were performed. Results of one representative experiment are shown.

FIG. 26 shows the steric clash of Bcr-Abl/T3151 with imatinib and pyrido-pyrimidines. Ribbon representation of the c-Abl kinase domain. Positions of imatinib and PD173955 towards the threonine at position 315 (as present in Bcr-Abl wild-type), and isoleucine 315 (as present in the Bcr-Abl mutant T3151) are shown. The extra hydrocarbon group in the side chain of the isoleucine may cause a steric clash not only with the phenylaminopyrimidine of imatinib, but also it may interfere with the dichlorphenyl substituent present in all pyrido-pyrimidine compounds that were tested. This figure was generated with PDB 11EP and PDB 1M52.

FIG. 27 shows the pharmacokinetic curve for PD166326 (po vs. iv).

FIG. 28 shows the pharmacokinetic curve for SKI-DV2-281 (po vs. iv).

FIG. 29 shows the pharmacokinetic curve for SKI-DV2-43 (po vs. iv).

FIG. 30 is a comparison of six of the most inhibitory pyrido-pyrimidine compounds of Bcr-Abl in inhibiting recombinant Abl and Src kinases in vitro.

FIG. 31 shows the percent inhibition of Abl protein tyrosine kinase activity in vitro by the eight most inhibitory pyrido-pyrimidine compounds in cellular assays.

### DESCRIPTION OF THE INVENTION

In recognition of the need to develop novel and effective kinase inhibitors as cancer therapies, the present invention provides novel compounds with such activity. In certain embodiments, the inventive compounds are useful in the treatment of cancer. In certain embodiments, these compounds have higher specificity, higher inhibitory potency, and/or increased solubility in aqueous solution.

General Description of Compounds of the Invention

The compounds of the invention include compounds of the general formula (O) as further defined below: 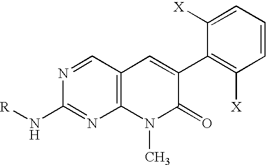
wherein each X is independently hydrogen, fluorine, chlorine, bromine, or iodine; and

R is a substituted or unsubstituted aliphatic, heteroaliphatic, aryl, heteroaryl, alkylaryl, or alkylheteroaryl moiety; and pharmaceutically acceptable derivatives thereof. In certain embodiments, X is chlorine. In certain embodiments, at least one X is chlorine. In other embodiments, both X are chlorine. In certain embodiments, R is a substituted or unsubstituted, cyclic aliphatic or heteroaliphatic moiety. In certain embodiments, R is a substituted or unsubstituted aryl or heteroaryl moiety. In certain embodiments, R is a substituted or unsubstituted carbocyclic aromatic moiety. In certain other embodiments, R is a substituted or unsubstituted heterocyclic aromatic moiety. In other embodiments, R is a substituted or unsubstituted five- or six-membered aromatic moiety. In other embodiments, R is phenyl or pyridinyl.

In certain embodiments, compounds of the formula (Oa) are provided: 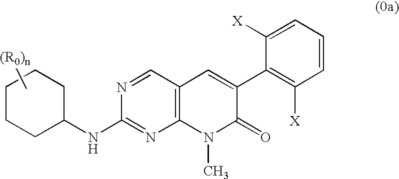
wherein each X is independently hydrogen, fluorine, chlorine, bromine, or iodine;
   n is 1, 2, 3, 4, or 5; and
   each R₀ is independently hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof. In certain embodiments, X is chlorine. In certain embodiments, n is 1, 2, or 3; in other embodiments, n is 1. In certain embodiments, R₀ is amino, hydroxy, hydroxymethyl, acetamido, iodo, fluoro, bromo, chloro, ethyl, propyl, cyclopropyl, butyl, cyclopbutyl, pentyl, hexyl, cyclohexyl, (6-biotinamido)hexamido, (2,3-dihydroxypropoxy)methyl, (2,3-dihydropropyl)amino, (acrylamido)phenylamido, or 4-methylpiperazinylcarboxy.

Compounds of formula (0) include:

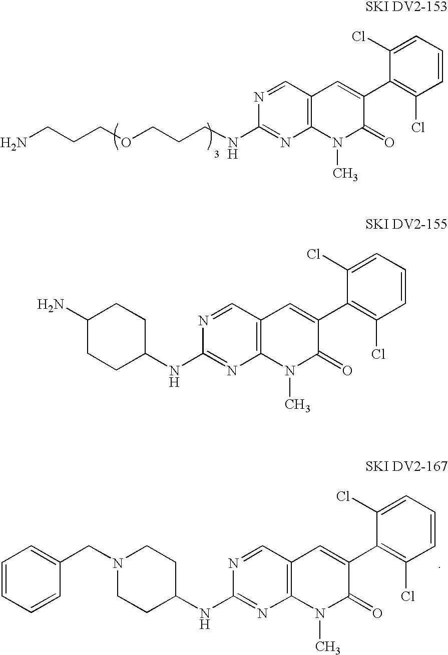

In certain embodiments, compounds of the formula (I) are provided: 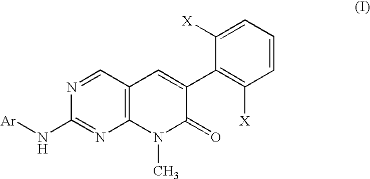
wherein each X is independently hydrogen, fluorine, chlorine, bromine, or iodine; and
Ar is a substituted or unsubstituted aryl, heteroaryl, alkylaryl, or alkylheteroaryl moiety; and pharmaceutically acceptable derivatives thereof. In certain embodiments, X is chlorine. In certain embodiments, Ar is a substituted or unsubstituted carbocyclic aromatic moiety. In certain other embodiments, Ar is a substituted or unsubstituted heterocyclic aromatic moiety. In other embodiments, Ar is a substituted or unsubstituted five- or six-membered aromatic moiety. In other embodiments, Ar is phenyl or pyridinyl.

In certain embodiments, compounds of the formula (II) are provided: 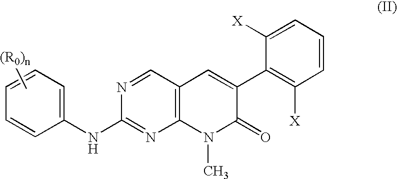
wherein each X is independently hydrogen, fluorine, chlorine, bromine, or iodine;
   n is 1, 2, 3, 4, or 5; and
   each R₀ is independently hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof. In certain embodiments, X is chlorine. In certain embodiments, n is 1, 2, or 3; in other embodiments, n is 1. In certain embodiments, R₀ is amino, hydroxy, hydroxymethyl, acetamido, iodo, fluoro, bromo, chloro, ethyl, propyl, cyclopropyl, butyl, cyclopbutyl, pentyl, hexyl, cyclohexyl, (6-biotinamido)hexamido, (2,3-dihydroxypropoxy)methyl, (2,3-dihydropropyl)amino, (acrylamido)phenylamido, or 4-methylpiperazinylcarboxy.

In certain embodiments, compounds of the formula (III) are provided: 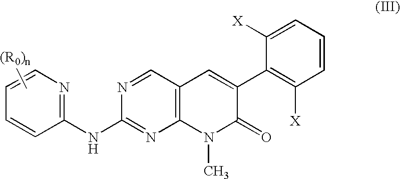
wherein each X is independently hydrogen, fluorine, chlorine, bromine, or iodine;
n is 1, 2, 3, 4, or 5; and
each R₀ is independently hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof. In certain embodiments, X is chlorine. In certain embodiments, n is 1, 2, or 3; in other embodiments, n is 1. In certain embodiments, R₀ is amino, hydroxy, hydroxymethyl, acetamido, iodo, fluoro, bromo, chloro, ethyl, propyl, cyclopropyl, butyl, cyclopbutyl, pentyl, hexyl, cyclohexyl, (6-biotinamido)hexamido, (2,3-dihydroxypropoxy)methyl, (2,3-dihydropropyl)amino, (acrylamido)phenylamido, or 4-methylpiperazinylcarboxy.

In other embodiments, compounds of the formula (IV) are provided 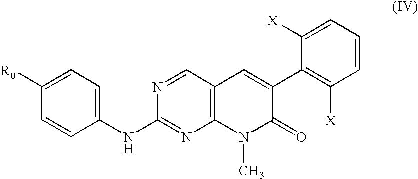
wherein each X is independently hydrogen, fluorine, chlorine, bromine, or iodine; and
R₀ is hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof. In certain embodiments, X is chlorine. In certain embodiments, R₀ is amino, hydroxy, hydroxymethyl, acetamido, iodo, fluoro, bromo, chloro, ethyl, propyl, cyclopropyl, butyl, cyclopbutyl, pentyl, hexyl, cyclohexyl, (6-biotinamido)hexamido, (2,3-dihydroxypropoxy)methyl, (2,3-dihydropropyl)amino, (acrylamido)phenylamido, or 4-methylpiperazinylcarboxy.

Compounds of formula (IV) include:

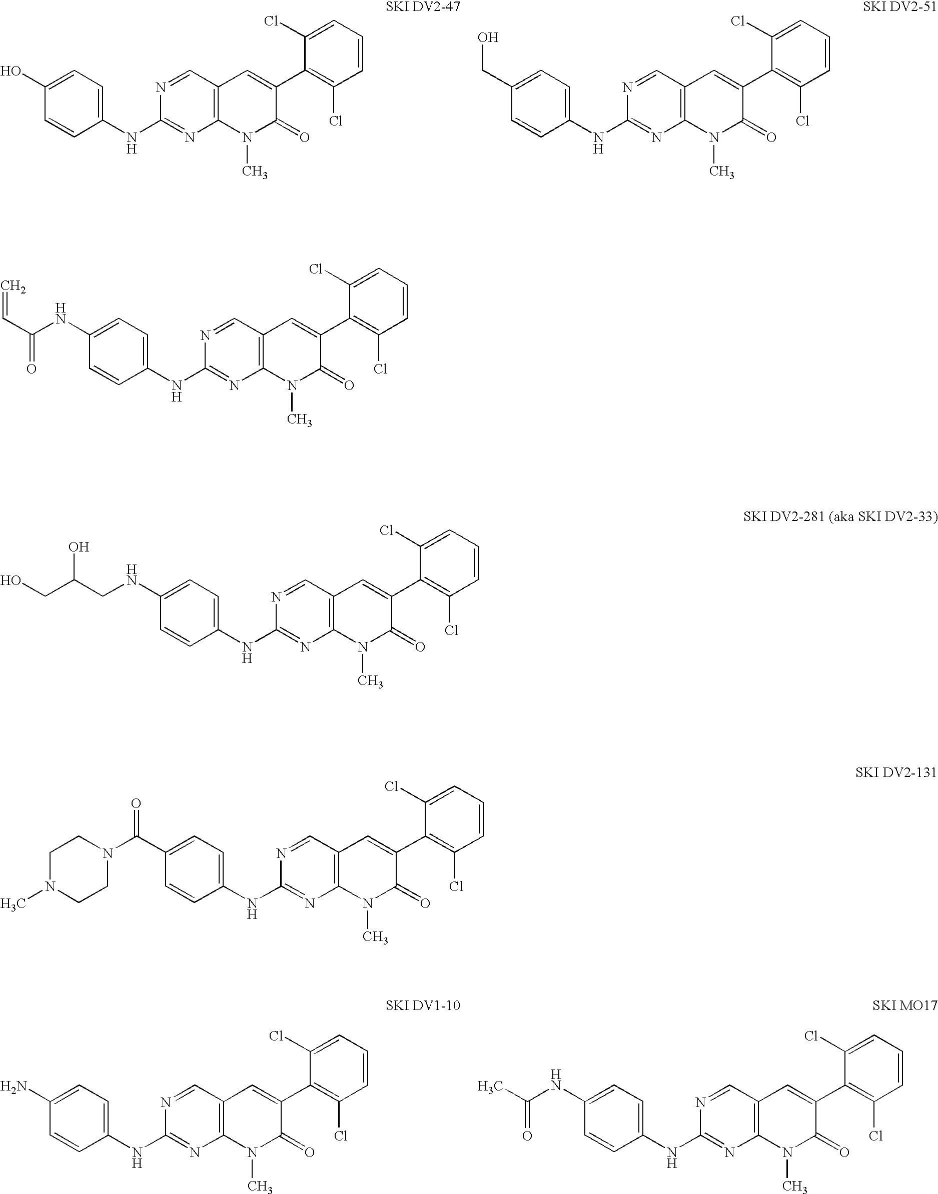

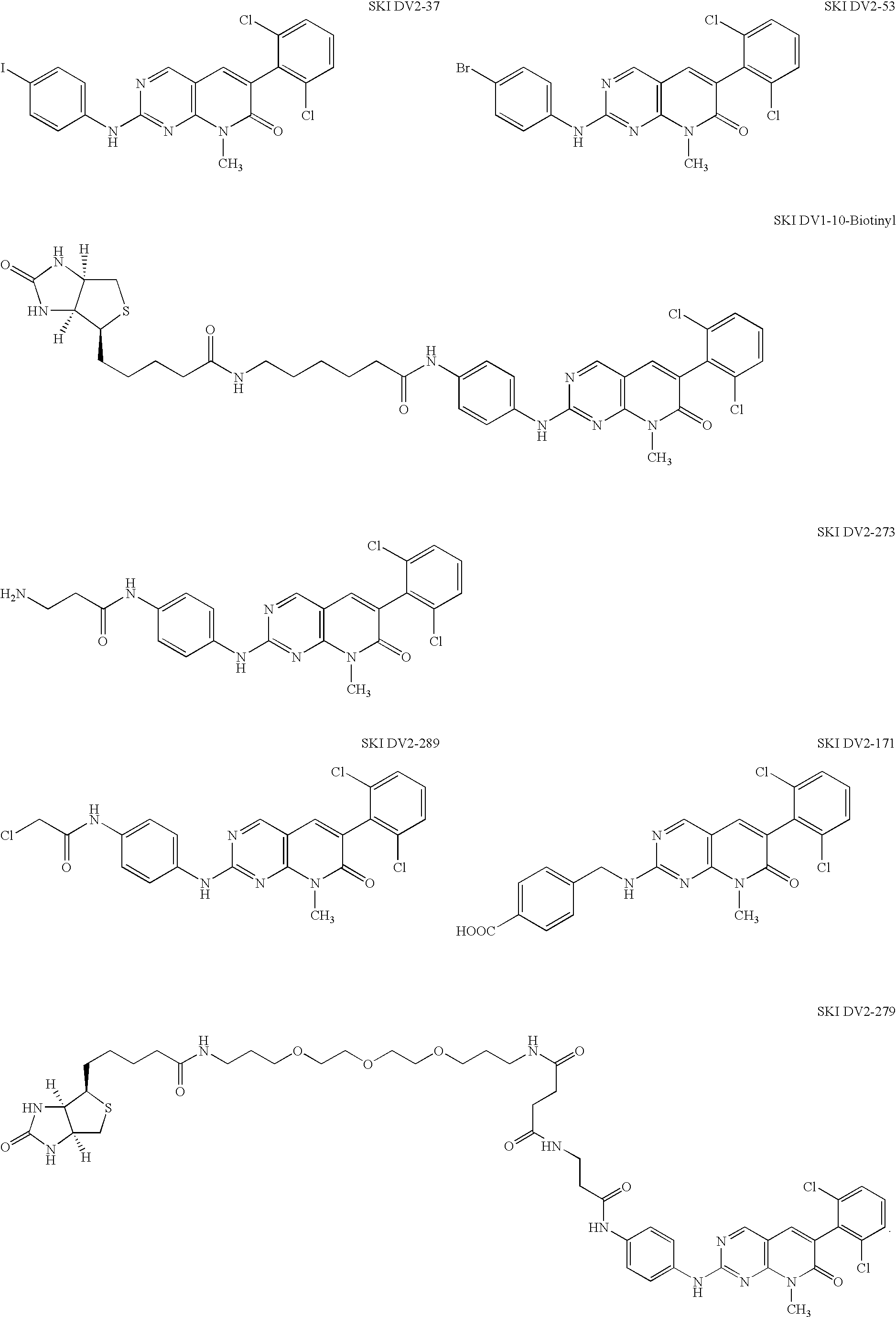

In other embodiments, compounds of the formula (V) are provided 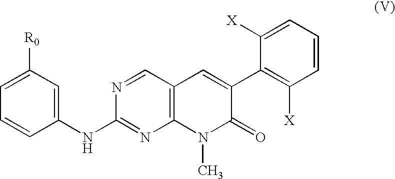
wherein each X is independently hydrogen, fluorine, chlorine, bromine, or iodine; and
   R₀ is hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof. In certain embodiments, X is chlorine. In certain embodiments, R₀ is amino, hydroxy, hydroxymethyl, acetamido, iodo, fluoro, bromo, chloro, ethyl, propyl, cyclopropyl, butyl, cyclopbutyl, pentyl, hexyl, cyclohexyl, (6-biotinamido)hexamido, (2,3-dihydroxypropoxy)methyl, (2,3-dihydropropyl)amino, (acrylamido)phenylamido, or 4-methylpiperazinylcarboxy.

Compounds of formula (V) include:

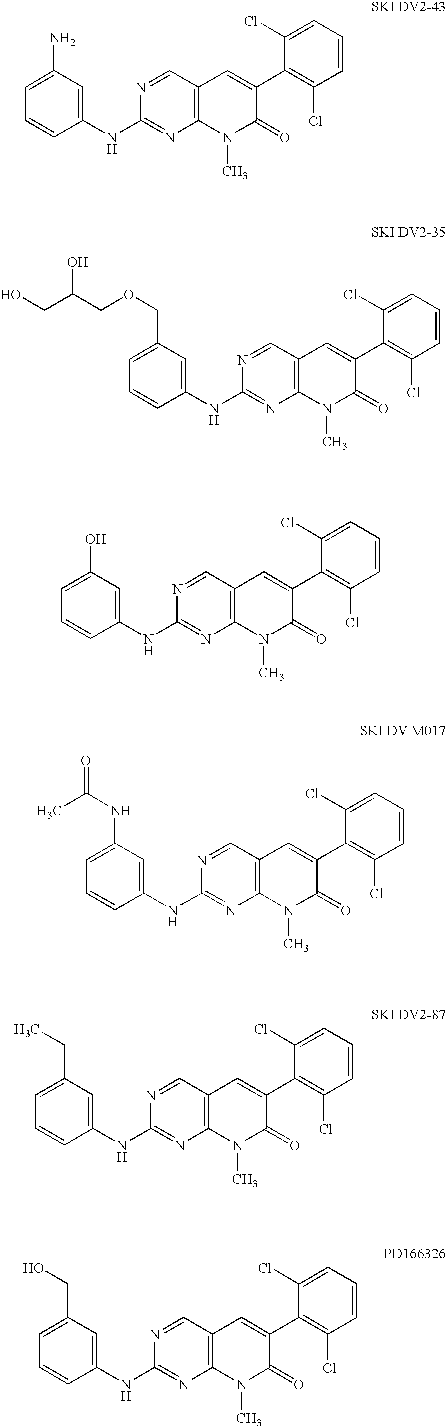

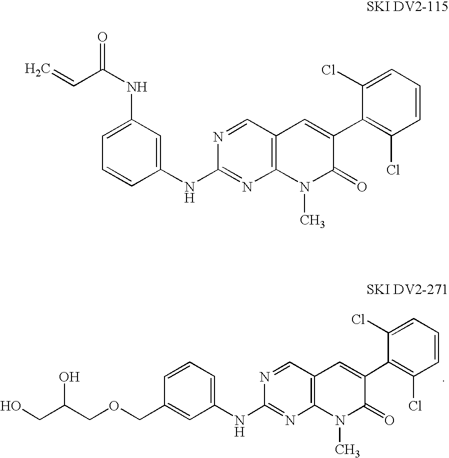

In other embodiments, compounds of the formula (VI) are provided 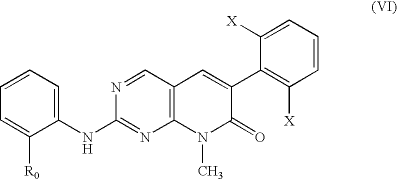
wherein each X is independently hydrogen, fluorine, chlorine, bromine, or iodine; and
   R₀ is hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof. In certain embodiments, X is chlorine. In certain embodiments, R₀ is amino, hydroxy, hydroxymethyl, acetamido, iodo, fluoro, bromo, iodo, ethyl, propyl, cyclopropyl, butyl, cyclopbutyl, pentyl, hexyl, cyclohexyl, (6-biotinamido)hexamido, (2,3-dihydroxypropoxy)methyl, (2,3-dihydropropyl)amino, (acrylamido)phenylamido, or 4-methylpiperazinylcarboxy.

In other embodiments, compounds of the formula (VII) are provided 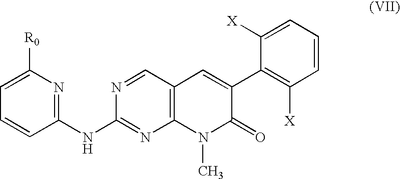
wherein each X is independently hydrogen, fluorine, chlorine, bromine, or iodine; and
   R₀ is hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof. In certain embodiments, X is chlorine. In certain embodiments, R₀ is amino, hydroxy, hydroxymethyl, acetamido, iodo, fluoro, bromo, chloro, ethyl, propyl, cyclopropyl, butyl, cyclopbutyl, pentyl, hexyl, cyclohexyl, (6-biotinamido)hexamido, (2,3-dihydroxypropoxy)methyl, (2,3-dihydropropyl)amino, (acrylamido)phenylamido, or 4-methylpiperazinylcarboxy.

Compounds of formula (VII) include:

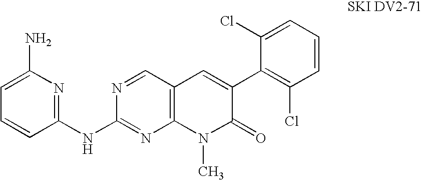

In other embodiments, compounds of the formula (VIII) are provided 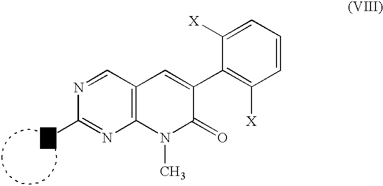
wherein each X is independently hydrogen, fluorine, chlorine, bromine, or iodine; and 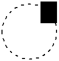
   is a substituted or unsubstituted heterocyclic moiety, wherein 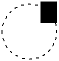
   is aromatic or nonaromatic; and pharmaceutically acceptable derivatives thereof. In certain embodiments, X is chlorine. In certain embodiments, 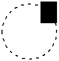
   is a pyrrolidine, piperidine, aziridine, azetidine, pyridine, pyrrole, oxazole, thiazole, indole, purine, carbazole, imidazole, isoxazole, pyrazole, or isothiazole moiety.

Compounds of formula (VIII) include:

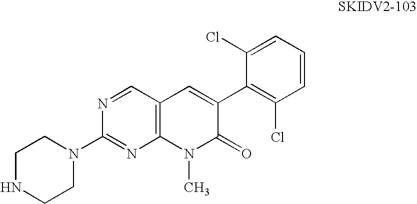

It will be appreciated that some of the foregoing classes and subclasses of compounds can exist in various isomeric forms. The invention encompasses the compounds as individual isomers substantially free of other isomers and alternatively, as mixtures of various isomers, e.g., racemic mixtures of stereoisomers. The invention also encompasses tautomers of specific compounds as described above. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of this invention which are of particular interest include those which:
exhibit cytotoxic or growth inhibitory effect on cancer cell lines maintained in vitro or in animal studies using a scientifically acceptable cancer cell xenograft model;
exhibit the ability to inhibit kinases, preferably specifically inhibit a kinase such as c-Abl or Bcr-Abl;
exhibit enhanced water solubility over other known kinase inhibitors such as ST1571 and PD173955, or additionally or alternatively exhibit sufficient solubility to be formulated in an aqueous medium; and
exhibit a therapeutic profile (e.g., optimum safety and curative effect) that is superior to that of known kinase inhibitors.

In another aspect, the present invention provides methods of identifying compounds which inhibit kinases and the compounds identified. In certain embodiments, these compounds discriminate between different kinases (e.g., between Abl and Src). In other embodiments, these compounds discriminate between different forms of the same kinase. The method includes contacting the compound with at least one kinase and determining whether the kinase activity of the protein is decreased. Comparing the inhibitory activity among several different kinases or forms of kinases would allow one to identify a compound which is selective. Compounds may also be identified based on co-crystallization studies and computer modeling. Compounds which interact with and specifically inhibit the target kinase through specific covalent or non-covalent interactions can be identified in this manner. For example, a greater number of hydrogen bonds and van der Waals interactions with the target kinase predicts a better inhibitor which is more specific. In addition, the identified compound can then be compared to the binding pocket of other kinases to further determine the selectivity of the identified compound.

This invention also provides a pharmaceutical preparation comprising at least one of the compounds as described above and herein, or a pharmaceutically acceptable derivative thereof, which compounds are capable of inhibiting the growth of or killing cancer cells, and, in certain embodiments of special interest are capable of inhibiting the growth of or killing multidrug resistant cancer cells.

The invention further provides a method for inhibiting tumor growth and/or tumor metastasis. In certain embodiments of special interest, the invention provides a method of treating cancers by inhibiting tumor growth and/or tumor metastasis for tumors containing multidrug resistant cancer cells. The method involves the administration of a therapeutically effective amount of the compound or a pharmaceutically acceptable derivative thereof to a subject (including, but not limited to a human or animal) in need of it. In certain embodiments, specifically for treating cancers comprising multidrug resistant cancer cells, the therapeutically effective amount is an amount sufficient to kill or inhibit the growth of multidrug resistant cancer cell lines. In certain embodiments, the inventive compounds are useful for the treatment of solid tumors.

3) Compounds and Definitions

As discussed above, this invention provides novel compounds with a range of biological properties. Compounds of this invention have biological activities relevant for the treatment of diseases or other disorders such as proliferative diseases, including, but not limited to cancer.

Compounds of this invention include those specifically set forth above and described herein, and are illustrated in part by the various classes, subgenera and species disclosed elsewhere herein

It will be appreciated by one of ordinary skill in the art that asymmetric centers may exist in the compounds of the present invention. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, a mixtures of stereoisomers or diastereomers are provided. Additionally, the invention encompasses both (Z) and (E) double bond isomers (or cis and trans isomers) unless otherwise specifically designated.

Additionally, the present invention provides pharmaceutically acceptable derivatives of the inventive compounds, and methods of treating a subject using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety that is susceptible to removal in vivo yielding the parent molecule as the pharmacologically active species. An example of a pro-drug is an ester that is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other funcational groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative disorders, including, but not limited to cancer. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl(propargyl), 1-propynyl and the like.

The term “alkoxy”, or “thioalkyl” as used herein refers to an alkyl group, as previously defined, attached to the parent molecular moiety through an oxygen atom or through a sulfur atom. In certain embodiments, the alkyl group contains 1-20 alipahtic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiments of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one, two or three of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of RX independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties that contain one or more oxygen, sulfur, nitrogen, phosphorus or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

“Labeled”: As used herein, the term “labeled” is intended to mean that a compound has at least one element, isotope or chemical compound attached to enable the detection of the compound. In general, labels fall into three classes: a) isotopic labels, which may be radioactive or heavy isotopes, including, but not limited to, ²H, ³H, ³²P, ³⁵S, ⁶⁷Ga, ^{99m}Tc (Tc-99m), ¹¹¹In, ¹²³I, ¹²⁵I, ¹⁶⁹Yb and ¹⁸⁶Re; b) immune labels, which may be antibodies or antigens; and c) colored or fluorescent dyes. It will be appreciated that the labels may be incorporated into the compound at any position that does not interfere with the biological activity or characteristic of the compound that is being detected. In certain embodiments of the invention, photoaffinity labeling is utilized for the direct elucidation of intermolecular interactions in biological systems. A variety of known photophores can be employed, most relying on photoconversion of diazo compounds, azides, or diazirines to nitrenes or carbenes (See, Bayley, H., Photogenerated Reagents in Biochemistry and Molecular Biology (1983), Elsevier, Amsterdam.), the entire contents of which are hereby incorporated by reference. In certain embodiments of the invention, the photoaffinity labels employed are o-, m- and p-azidobenzoyls, substituted with one or more halogen moieties, including, but not limited to 4-azido-2,3,5,6-tetrafluorobenzoic acid.

“Polymer”: The term “polymer”, as used herein, refers to a composition comprising chains that may be open, closed, linear, branched or cross-linked of repeating units (monomers) that may be the same or different. It will be appreciated that in certain embodiments the term polymer refers to biopolymers, which, as used herein, is intended to refer to polymeric materials found in nature or based upon those materials found in nature, including, but not limited to nucleic acids, peptides, and mimetics thereof. In certain other embodiments, the term polymer refers to synthetic polymers, such as biodegradable polymers or other polymeric materials. It will be appreciated that polymeric solid supports are also encompassed by the polymers of the present invention. Inventive compounds can be attached to polymeric supports and thus certain synthetic modifications can be conducted on the solid phase. As used herein, the term “solid support” is meant to include, but is not limited to, pellets, disks, capillaries, hollow fibers, needles, pins, solid fibers, cellulose beads, pore-glass beads, silica gels, polystyrene beads optionally cross-linked with divinylbenzene, grafted co-poly beads, poly-acrylamide beads, latex beads, dimethylacrylamide beads optionally crosslinked with N-N′-bis-acryloylethylenediamine, and glass particles coated with a hydrophobic polymer. One of ordinary skill in the art will realize that the choice of particular solid support will be limited by the comparability of the support with the reaction chemistry being utilized. An exemplary solid support is a Tentagel amino resin, a composite of 1) a polystyrene bead crosslinked with divinylbenzene and 2) PEG (polyethylene glycol). Tentagel is a particularly useful solid support because it provides a versatile support for use in on-bead or off-bead assays, and it also undergoes excellent swelling in solvents ranging from toluene to water.

4) Synthetic Methodology:

The synthesis of compounds of the invention are described below in the Examples. General synthetic methodologies are included in Klutchko et al. J. Med Chem. 41(17):3276-92, Aug. 13, 1998, and Boschelli et al. J. Med Chem. 41(22):4365-77, Oct. 22, 1998; each of which is incorporated herein by reference. In recognition of the need for improved or additional synthetic methodologies to efficiently generate the inventive compounds and analogues thereof in large quantities, the present invention provides an efficient and modular route for the synthesis of the inventive compounds and analogues thereof. Although the synthesis of certain exemplary compounds is described in the Exemplification herein, it will be appreciated that this methodology is generally applicable to the generation of analogues and conjugates as discussed above for each of the classes and subclasses described herein.

5) Uses, Formulation and Administration

Pharmaceutical Compositions

As discussed above, the present invention provides novel compounds having antitumor and antiproliferative activity, and thus the inventive compounds are useful for the treatment of cancer. Accordingly, in another aspect of the present invention, pharmaceutical compositions are provided, wherein these compositions comprise any one of the compounds as described herein, and optionally comprise a pharmaceutically acceptable carrier. In certain embodiments, these compositions optionally further comprise one or more additional therapeutic agents. In certain other embodiments, the additional therapeutic agent is an anticancer agent, as discussed in more detail herein.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-cancer compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; Cremophor; Solutol; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds and Pharmaceutical Compositions

In yet another aspect, according to the methods of treatment of the present invention, tumor cells are killed, or their growth is inhibited by contacting said tumor cells with an inventive compound or composition, as described herein. Thus, in still another aspect of the invention, a method for the treatment of cancer is provided comprising administering a therapeutically effective amount of an inventive compound, or a pharmaceutical composition comprising an inventive compound to a subject in need thereof, in such amounts and for such time as is necessary to achieve the desired result. In certain embodiments of the present invention a “therapeutically effective amount” of the inventive compound or pharmaceutical composition is that amount effective for killing or inhibiting the growth of tumor cells. For example, the amount effective to kill 50%, 90%, 95%, or 99% of the cells in a cell culture such as described below in the Examples. The compounds and compositions, according to the method of the present invention, may be administered using any amount and any route of administration effective for killing or inhibiting the growth of tumor cells. Thus, the expression “amount effective to kill or inhibit the growth of tumor cells”, as used herein, refers to a sufficient amount of agent to kill or inhibit the growth of tumor cells. The exact amount required will vary from subject to subject, depending on the species, age, and general condition of the subject, the severity of the infection, the particular anticancer agent, its mode of administration, and the like. The anticancer compounds of the invention are preferably formulated in dosage unit form for ease of administration and uniformity of dosage. The expression “dosage unit form” as used herein refers to a physically discrete unit of anticancer agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments of the invention, the inventive compounds as described herein are formulated by conjugating with water soluble chelators, or water soluble polymers such as polyethylene glycol as poly(1-glutamic acid), or poly(1-aspartic acid), as described in U.S. Pat. No. 5,977,163, the entire contents of which are hereby incorporated by reference. In certain embodiments, the compounds of the invention may be administered orally or parenterally at dosage levels of about 0.01 mg/kg to about 100 mg/kg, preferably from about 0.1 mg/kg to about 50 mg/kg, and more preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As discussed above, the compounds of the present invention are useful as anticancer agents, and thus may be useful in the treatment of cancer, by effecting tumor cell death or inhibiting the growth of tumor cells. In general, the inventive anticancer agents are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer, to name a few. In certain embodiments, the inventive anticancer agents are active against leukemia cells and melanoma cells, and thus are useful for the treatment of leukemias (e.g., myeloid, lymphocytic, myelocytic and lymphoblastic leukemias) and malignant melanomas. In still other embodiments, the inventive anticancer agents are active against solid tumors and also kill and/or inhibit the growth of multidrug resistant cells (MDR cells).

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

For example, other therapies or anticancer agents that may be used in combination with the inventive anticancer agents of the present invention include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In another aspect, the present invention provides combination comprising compound of the invention and medication known to combat the side effects of these compounds. For example, medication which relieves pain, anemia, nausea, hair loss, lethargy, etc. may be combined with the inventive compounds in a therapeutic combination. In particular, pain or nausea medication may be combined with the inventive compounds.

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### EXEMPLIFICATION

### Example 1

### Crystal Structures of the Kinase Domain of c-Abl Complexes to Small Molecule Inhibitors and Design of Better c-Abl Inhibitors

Materials and Methods

Protein Expression and Purification. The gene encompassing the kinase domain of murine c-Abl (residues 229-515) was cloned into a plasmid in which a hexa-histidine tag is fused to the N-terminus of the protein (pFastbac HTa, Gibco BRL). Due to a cloning artefact the construct contains an additional six amino acids (sequence GAMDPS) at the N-terminus. This recombinant plasmid was transformed into E. coli (DH10Bac), containing bacmid DNA and helper plasmid. After transposition between donor plasmid and bacmid has taken place (˜3-5 hrs), single colonies containing recombinant plasmid were identified and expanded. Bacmid DNA containing the Abl insert was isolated and transfected into Sf9 insect cells. Bacculovirus obtained from the transfection was then used to infect Sf9 cells grown in suspension to a density of 2.5×10⁶ cells per ml at a multiplicity of infection of 10. Cells were grown for 48 hours, centrifuged, and the pellet stored at −80° C. Cells were thawed, resuspended in buffer A (50 mM Tris/HCl pH 8.0, 10% glycerol, 15 mM β-mercaptoethanol) and lysed by sonication. The resulting suspension was diluted (1:2) and centrifuged at 18000 rpm for 1 hour. The supernatant was filtered and loaded onto a 65 ml Q-sepharose ion-exchange column equilibrated in buffer A. Protein was eluted from the column with a linear salt gradient (0-1 M NaCl). Fractions containing Abl protein (identified by anti-hexa-histidine Western blot) were pooled, loaded onto a Ni-NTA column (Qiagen) equilibrated in 20 mM Tris/HCl pH 8.0, 500 mM NaCl, 5% glycerol, 20 mM imidazole and 5 mM β-mercaptoethanol. The protein was eluted with a linear imidazole gradient (20 mM-1M). Abl-containing fractions were pooled and incubated with Tobacco Etch Virus protease overnight at 4° C. in order to cleave the hexa-histidine tag from Abl. Next, inhibitor compound (PD173955 or STI-571) dissolved in DMSO was added at 3 times the molar protein concentration with constant stirring at 4° C. The inhibitor-protein complex was then concentrated and loaded onto a Sephadex 75 gel filtration column (HiLoad 16/60) equilibrated in 20 mM Tris/HCl pH 8.0, 100 mM NaCl, 3 mM DTT. Abl:inhibitor complex containing fractions were pooled and concentrated to 10 mg/ml. Yersinia protein tyrosine phosphatase (YopH) used for enzymatic analysis was expressed as a GST-fusion in E. coli and purified on a GST-column.

Synthesis of STI-571 and PD173955. STI-571 and PD173955 were synthesized and purified in the Organic Synthesis Core Facility at MSKCC as described respectively in Buchdunger et al. J. Pharmacol. Exp. Ther. 295:139-145, 2000, and Kraker et al. Biochem. Pharmacol. 60:885-898, 2000; each of which is incorporated herein by reference. The compounds were dissolved as 20 mM aliquots in DMSO and stored at −80° C. until needed.

Crystallization and Data Collection. Using the hanging drop vapour diffusion method (1 μl protein solution +1 μL reservoir solution) in a sparse matrix screen, crystals of the AblK:STI-571 complex grew in 25% (w/v) PEG 4000, 100 mM MES/NaOH buffer pH 6.5, 0.2 M MgCl₂ at 4° C. (space group F222 with a=112.9 Å, b=147.4 Å, c=153.4 Å and two molecules in the asymmetric unit) and those of AblK:PD173955 in 12% (w/v) PEG 20000, 100 mM MES/NaOH buffer pH 6.5 at 20° C. (space group P2₁2₁2 with a=115.8 Å, b=125.7 Å, c=56.7 Å and two molecules in the asymmetric unit). The crystal form for AblK:STI-571 is similar to that reported previously for the variant of STI-571 (Schindler et al. Science 289:1938-1942, 2000; incorporated herein by reference). The crystals were cryoprotected with the addition of 20% (v/v) ethylene glycol or 20% glycerol (v/v) for AblK:STI-571 and AblK:PD173955, respectively. X-ray diffraction data were collected on crystals flash frozen in liquid propane stored in liquid nitrogen at the Advanced Light Source (beamline 5.0.2) for AblK:PD173955 and CHESS (beamline F1) for AblK:STI-571. All data were integrated and scaled with DENZO and Scalepack (Otwinowski et al. Methods in Enzymology 276:307-326, 1997; incorporated herein by reference).

Structure Determination and Refinement. The structure of AblK:STI-571 was solved by computing a difference Fourier electron density map after rigid-body refinement of the previously determined structure (PDB entry 1FPU), from which the inhibitor was removed. The structure of AblK:PD173955 was solved by molecular replacement with the program AMORE (Navaza Acta Crystallogr A. 50:157-163, 1994; incorporated herein by reference), using one molecule of Abl (PDB code 1FPU), without inhibitor, as a search model. Both models were subsequently refined with the program CNS (Brunger et al. Acta Crystallogr D. Biol. Crystallogr 54(Pt. 5):905-921, 1998; incorporated herein by reference) using simulated annealing, conjugate gradient least-squares minimization and individual B-factor refinement with intervening rounds of manual model building using 0 (Jones et al. Acta Crystallogr.-Section A-Foundations of Crystallography 54(Pt. 5):905-921, 1998; incorporated herein by reference) making extensive use of simulated annealing omit maps. With AbIK:PD173955, noncrystallographic symmetry (NCS) restraints were maintained and only two B-factors per residue were applied until the final round of refinement, at which point NCS restraints were released and individual B-factors were refined.

Kinase Assays. We used a continuous spectrophotometric kinase assay to measure the kinase activity of the catalytic domain of Abl. In this assay, ADP that is produced as a result of phosphorylation by the enzyme is coupled to the oxidation of NADH to NAD⁺, which produces a decrease in absorbance at 340 nm (Barker et al. Biochemistry 34:14843-14851, 1995; incorporated herein by reference). The assays were carried out in 100 mM Tris/HCl pH 7.5, 10 mM MgCl₂, 0.5 mM ATP, 1 mM phosphoenolpyruvate, 0.28 mM NADH, 89 units/ml pyruvate kinase, 124 units/ml lactate dehydrogenase, 4% DMSO, and 0.5 mM peptide substrate (AEEEIYGEFEAKKKKG) at 30° C. in a 150 μL reaction volume. Reactions were initiated by the addition of 10 nM phosphorylated Abl (pAbl) to the mix containing varying amounts of STI-571 or PD173955. After the activity of pAbl was measured, 0.8 μM YopH (Yersinia protein tyrosine phosphatase) was added to measure the effect of the inhibitors on unphosphorylated Abl in situ. YopH treatment had no effect on the coupling enzymes in the assay, nor did it significantly change the basal activity of pAbl in the absence of inhibitors. Because our construct showed very inefficient autophosphorylation levels, we used Src to phosphorylate Abl on the activation loop (Schindler et al. Science 289:1938-1942, 2000; Warmuth et al. J. Biol. Chem. 272:33260-33270, 1997; Plattner et al. Genes Dev. 13:2400-2411, 1999; each of which is incorporated herein by reference) by incubating 1 λM Abl with 50 nM of activated c-Src (Gibco BRL) in buffer containing 100 mM Tris/HCl pH 7.5, 30 mM MgCl₂ and 10 mM ATP for 2 hours at 4° C.

Results and Discussion

Overall features of the structures. The three-dimensional structures of the kinase domain of Abl in complex with STI-571 and PD173955 were determined at 2.1 Å and 2.6 Å, respectively. Both structures are of the unphosphorylated form of Abl. In both crystal forms there are two independent molecules in the crystallographic asymmetric unit. The relationship between the pairs of molecules in the two crystal forms is unrelated, and the crystal structures do not imply that the Abl kinase forms any particular kind of dimeric structure. The overall structure of the kinase domain in the two crystal forms is very similar, with the typical bilobal architecture that is conserved amongst eukaryotic Ser/Thr and tyrosine kinases. Residues 225 to 350 (mouse c-Abl spliceform I numbering) make up the N-terminal lobe of the kinase (N-lobe) and the residues 354 to 498 comprise the C-terminal lobe (C-lobe). The amino acid sequence of murine Abl differs from that of human Abl by one residue.

The conformation of the activation loop is very different in the STI-571 and PD173955 complexes. There is one region in which the structure of the kinase domain differs markedly between the STI-571 and PD173955 complexes, and this is restricted to the activation loop (residues 381 to 402 in Abl), a centrally located regulatory element in protein kinases.

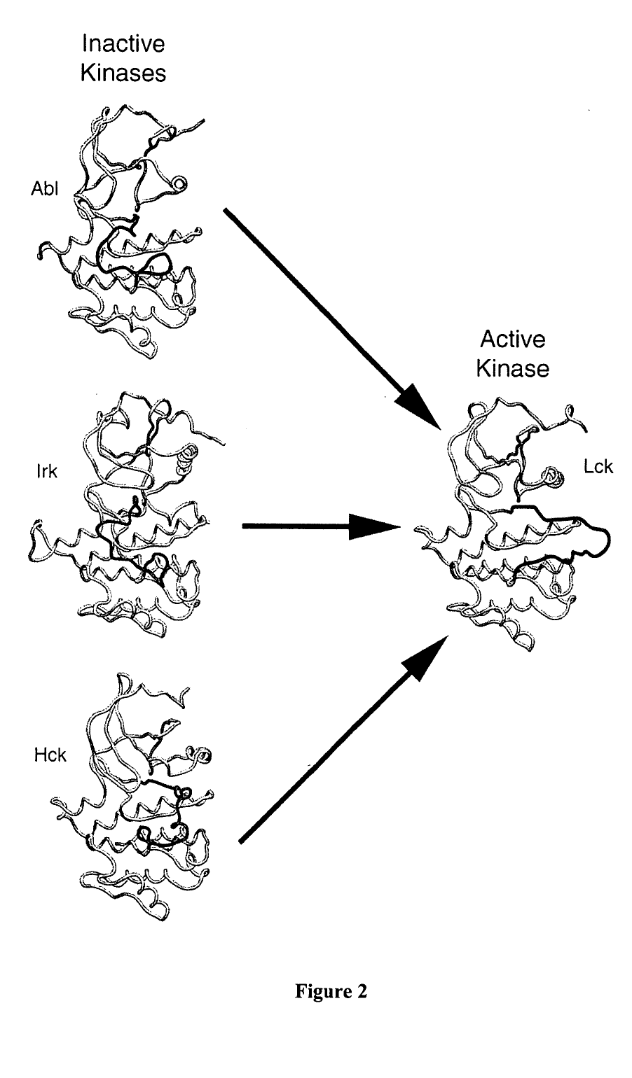

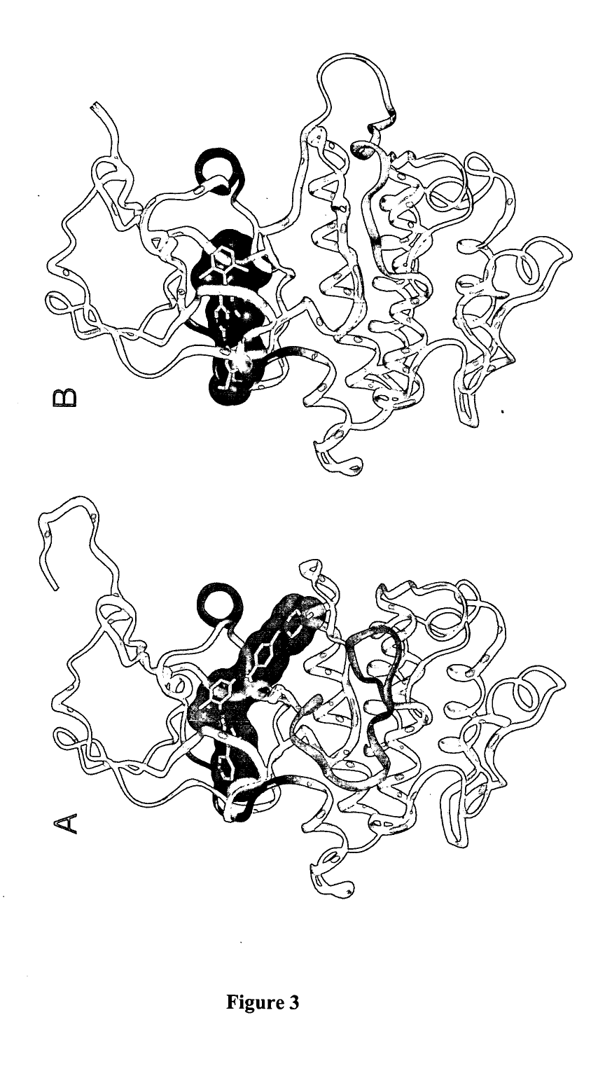

While the conformations of protein kinases that are fully active are very similar, there are striking differences in the inactive conformations of kinases from different sub-families (FIG. 2). These differences include alterations in the inter-lobe orientation and the disposition of helix αC in the N-lobe. A crucial aspect of the conformational transition between the active and inactive states is the activation loop or segment, which is of varying length and sequence, and is often the site of activating phosphorylation in the kinase domain.

In structures of protein kinases that are in a fully active state, the activation loop is in an extended or open conformation. There are two crucial aspects to this “active” conformation of the activation loop. First, an aspartic acid residue (Asp 381 in Abl) within a strictly conserved Asp-Phe-Gly (DFG) motif at the N-terminal base of the activation loop is positioned so as to interact properly with a magnesium ion that coordinates the phosphate groups of ATP. Second, the rest of the loop is positioned away from the catalytic center so that the C-terminal portion of the activation loop provides a platform for substrate binding.

In the structure of the AblK:STI-571 complex, the conformation of the activation loop is essentially the same as that seen in the previously determined structure of Abl complexed to the STI-571 variant (Schindler et al. Science 289:1938-1942, 2000; incorporated herein by reference) (FIG. 3A). The N-terminal portion of the activation loop, including the DFG motif, is rotated drastically with respect to the active conformation so that it is Phe 382 of the DFG motif that points towards the ATP binding site rather than Asp 381. As we discuss below, this altered conformation of Phe 382 is crucial for the proper binding of STI-571. The rest of the activation loop adopts a conformation in which the region surrounding Tyr 393 (the site of activating phosphorylation that is unphosphorylated in this structure) mimics a substrate binding to the enzyme, thereby blocking the active site.

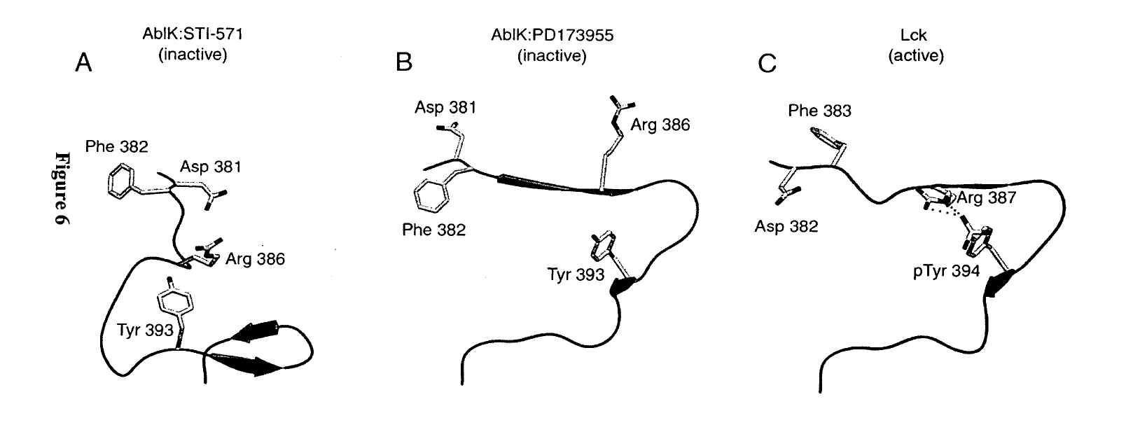

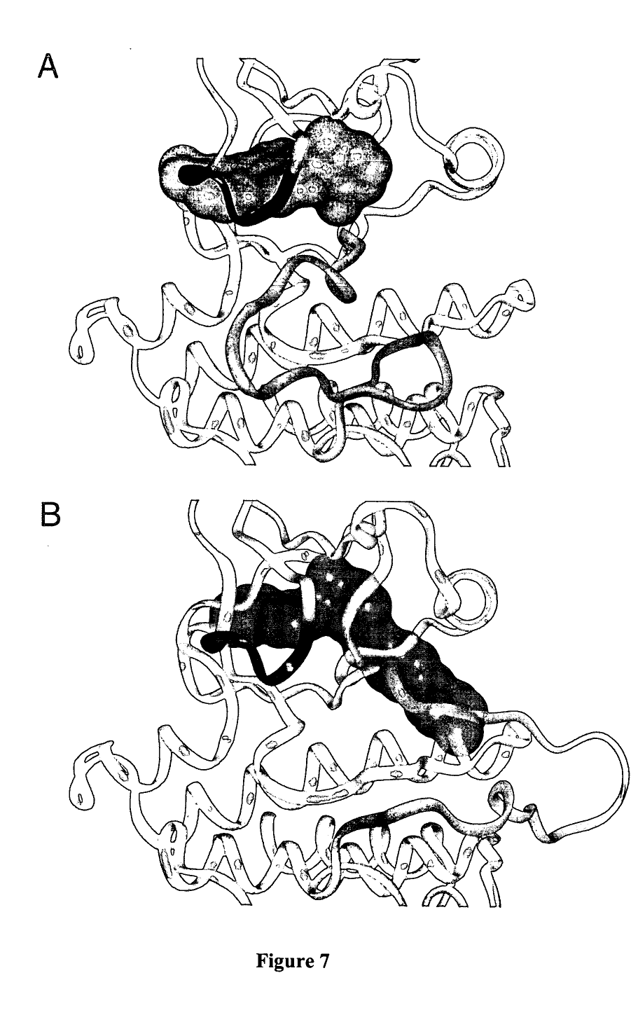

In the AblK:PD173955 complex, most of the activation loop is in a conformation very similar to that seen in active protein kinases, leaving the catalytic center of the enzyme unblocked (FIG. 3B). The conformation of the DFG motif is different from that seen in active kinases (FIG. 6), and it is likely that this is a consequence of the unphosphorylated state of the enzyme. Detailed examination of the structure (see below) indicates that the binding of PD173955 is insensitive to whether the activation loop is in the fully active conformation or not. This is in contrast to STI-571, whose binding to the kinase domain is blocked by the active conformation of the activation loop.

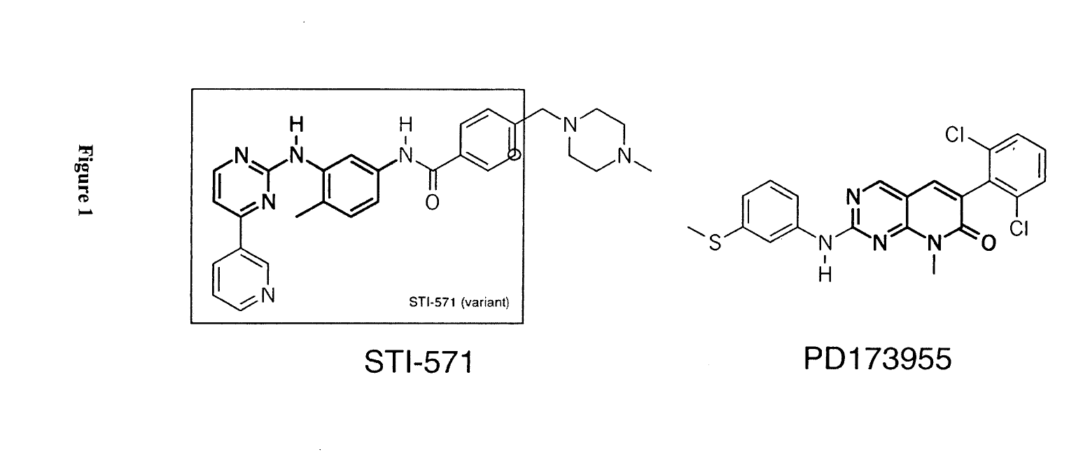

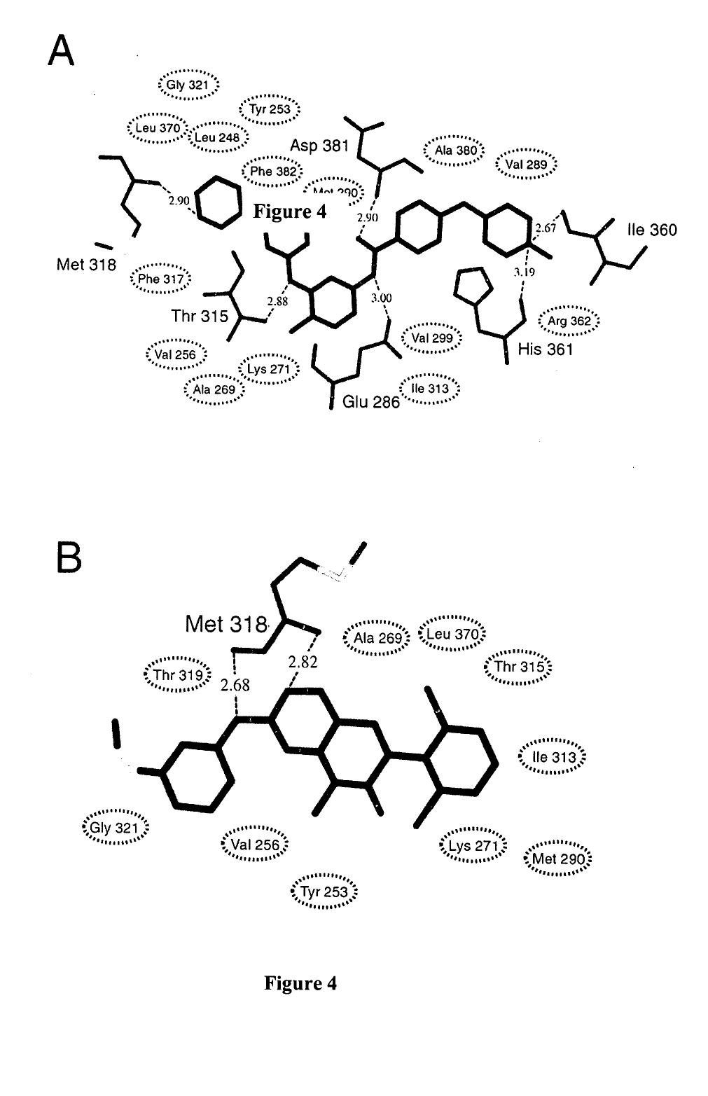

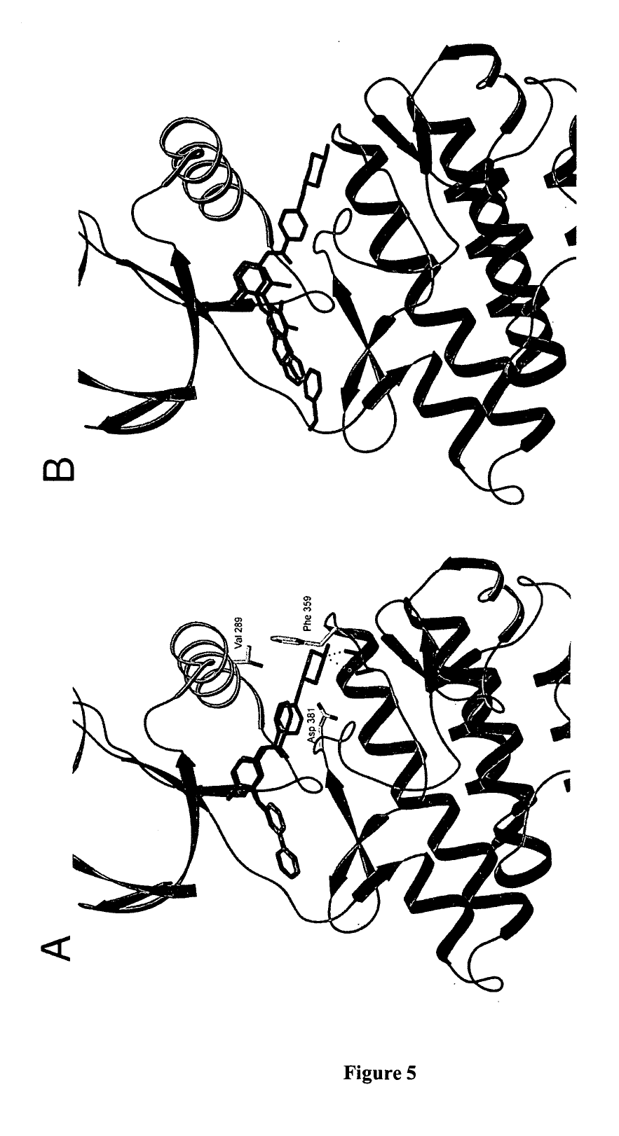

Structure of Abl kinase domain bound to STI-571. STI-571 is derived from a 2-phenylaminopyrimidine scaffold. With reference to the orientation shown in FIG. 1A, STI-571 consists of the core scaffold (bold lines) plus a pyridine substituent on the bottom-left side and a peptide bond followed by a phenyl ring and a piperazinyl ring to the right of the core. The drug is sandwiched between the N- and C-lobes of the kinase domain and penetrates through the central region of the kinase, from one side to the other (FIG. 3A). Only the leftmost part of STI-571 (pyridine and pyrimidine rings) occludes the region where the adenine ring of ATP normally binds. The rest of the compound penetrates further into the hydrophobic core of the kinase and wedges itself between the activation loop and helix αC, freezing the kinase in an inactive conformation. In total, the compound makes 6 hydrogen bonds with the protein, and the majority of contacts are mediated by van der Waal interactions (FIG. 4A). A total of 125I A² of surface area is buried between the drug and the protein.

The adenine group of ATP normally makes two hydrogen bonds with backbone atoms of the peptide chain connecting the N- and C-lobes of kinase domains (Zheng et al. Biochemistry 32:2154-2161, 1993; incorporated herein by reference). The extracyclic amino group of ATP donates a hydrogen bond to the carbonyl oxygen of the residue corresponding to Glu 316 in Abl, and nitrogen N1 of the purine ring accepts a hydrogen bond from the amide nitrogen of residue Met 318. Most small molecule inhibitors of protein kinases (including PD173955, see below) are anchored to the kinase domain by a pair of hydrogen bonds that mimic those formed by adenine. The pattern of hydrogen bonds formed by STI-571 and the previously analyzed variant (Schindler et al. Science 289:1938-1942, 2000; incorporated herein by reference) are similar and both lack a direct hydrogen bond to the inter-lobe connector, corresponding to that formed by the amino group of adenine (a water-mediated hydrogen bond is formed instead). This results in STI-571 being bound more towards the mouth of the kinase domain (that is, displaced away from the inter-lobe connector) than is typically the case for small molecule inhibitors.

The outward displacement of STI-571 appears to be facilitated by the formation of a hydrophobic cage around the pyridine and pyrimidine rings systems of the drug. This cage is formed by a distorted structure adopted by the phosphate binding P-loop and by the activation loop of c-Abl. The P-loop, comprised of the first two β-strands of the N-lobe and the loop connecting them, is normally in an extended conformation. In the STI-571 complex (and in the complex with PD173955, see below) the loop adopts a compact structure which moves Tyr 253 over into close contact with the inhibitor. The hydrophobic cage is completed by resides Leu 370 and Phe 382. A similar hydrophobic cage has been seen previously in the structure of the FGF receptor bound to an inhibitor (Mohammadi et al. Science 276:955-960, 1997; incorporated herein by reference).

The structure of the AblK:STI-571 complex is essentially the same as the structure of AblK:STI-571 (variant). The ‘left’ half of the compound overlaps almost exactly but tilts slightly relative to the variant, beginning at the rightmost phenyl ring (FIG. 5A). The additional piperazinyl ring (not present in the variant) lies along a partially hydrophobic pocket on the surface, making van der Waals interactions with Val 289, Phe 359 and Asp 381, and hydrogen bonds with the carbonyl oxygen atoms of Ile 360 and His 361. The presence of the piperazinyl ring does not change the conformation of the activation loop and the mechanism of STI-571 inhibition derived from the AblK:STI-571 (variant) structure remains unchanged.

The kinase domain of c-Abl is about 47% identical in sequence to that of the Src family of tyrosine kinases. Src kinases cannot bind STI-571 even though most of the residues that make contact with the drug in c-Abl are conserved in Src kinases (Druker et al. Nat. Med. 2:561-566, 1996; incorporated herein by reference). One explanation for this may be due to the differences observed in the inactive conformations of Src and Abl (FIG. 2). It is possible that the activation loop of Src cannot adopt the conformation seen in inactive Abl, although why this is so is unclear. In both Src and Abl, the unphosphorylated activation loop positions the site of activating tyrosine phosphorylation (Tyr 416 in Src, Tyr 393 in Abl) such that the tyrosine sidechain points into the active site, blocking it. The difference is that in the c-Abl structure the central portion of the activation loop closely mimics the binding of a peptide substrate to the kinase, whereas in Src kinases it does not. The conformation of the activation loop in Abl, including the peptide substrate-mimicking element, resembles in general terms the activation loop of unphosphorylated insulin receptor kinase (IRK) (Hubbard et al. Nature 372:746-754, 1994; incorporated herein by reference. Despite this similarity IRK is not able to bind STI-571 (Druker et al. Nat. Med. 2:561-566, 1996; incorporated herein by reference). A simple explanation for this is that Thr 315, which makes an important hydrogen bond with STI-571 in the c-Abl complex, is replaced by a bulkier methionine residue in IRK, which would occlude the STI-571 binding site. STI-571 binding is also prevented by a number of subtle differences in the positions of key residues in insulin receptor kinase that are likely to prevent the accommodation of STI-571. For example, Phe 1151 of the DFG motif in inactive IRK completely overlaps the pyrimidine ring of STI-571. Additionally, there are clashes between the piperazinyl ring and Phe 1128 of the catalytic loop. Finally, the conformations of the P-loops in IRK and Abl are quite distinct and this causes Lys 1030 of IRK to collide with the compound.

Structure of Abl kinase domain bound to PD173955. PD173955 is based on the pyrido-[2,3-d]pyrimidine series of compounds where the name refers to the central bicyclic ring of the compound (FIG. 1B). PD173955 has two substituents: an amino phenyl methyl sulfide on the left and a dichlorophenyl on the right, with reference to the orientation in FIG. 1B. PD173955 binds between the N- and C-lobes of the kinase domain. It is, however, a smaller molecule than STI-571 and does not reach anywhere near as deeply into the kinase domain (FIGS. 3B and 5B). The pyrido-pyrimidine ring occupies the place of the pyrimidine and pyridine rings of STI-571, with the additional phenyl and methyl thioether extending outwards to solvent. The dichlorophenyl ring occupies a similar position to the core phenyl ring of STI-571. Both compounds have this overlapping phenyl ring rotated such that the plane of the ring is perpendicular to the preceding parts of the compounds. The majority of the interactions between PD173955 and the protein are also mediated by van der Waal interactions (FIG. 4B). Met 318, which hydrogen bonds to STI-571, also hydrogen bonds to PD173955, again through backbone interactions. These are the only hydrogen bonds that PD173955 makes with the protein. Thr 315, which makes an important hydrogen bond with STI-571 only makes van der Waals interactions with PD173955. The two chlorine atoms on the phenyl ring of PD173955 are buried inside the protein, making several van der Waals interactions with Val 256, Ala 269 and Ala 381. The methyl thioether on the other end of the compound is solvent exposed. A total of 913 Å² of surface area on the inhibitor is buried in the complex.

Conformation of Abl kinase domain in complex with PD173955. The overall conformation of the kinase domain, including the inter-lobe orientation and the conformation of helix αC are essentially identical in the STI-571 and PD173955 complexes. However, as noted earlier, the conformations of the activation loops are dramatically different (FIG. 3 and FIG. 6). Whereas in the AblK:STI-571 structure this loop is folded in towards the protein (closed) and mimics substrate binding, in the AblK:PD173955 structure the loop protrudes outward in an extended conformation (open) and resembles that of an activated kinase (Yamaguchi et al. Nature 384:484-489, 1996; incorporated herein by reference). Beginning at the highly conserved Asp-Phe-Gly (DFG) motif, the loops of AblK:STI-571 and AblK:PD173955 diverge. Asp 381 of AblK:STI-571 continues inwards towards the protein, whereas in AblK:PD173955 it extends away from the protein. The loops then meet again at Pro 402.

Although the open conformation of the activation loop seen here resembles that seen in the structure of active kinases such as Lck, the DFG motifs are quite different (FIGS. 6B and 6C). Again, beginning at Asp 381 (Asp 382 in Lck), the residues kink in opposite directions such that Phe 383 in Lck occupies the space of Asp 381 in AblK:PD173955 and vice versa. Following this, the loops follow essentially the same path. The sites of phosphorylation in the activation loops, Tyr 393 in Abl and Tyr 394 in Lck, have essentially the same conformation. In Lck, Tyr 394 is phosphorylated and makes critical hydrogen bonds with the sidechain of Arg 387, which presumably stabilizes the open conformation. In Ablk:PD173955, Tyr 393 is not phosphorylated and does not make any hydrogen bonds. Arg 386 (equivalent to Arg 387 in Lck) has weak electron density, points into the solvent and does not contribute to the stabilization of the open conformation. This suggests that the c-Abl kinase domain, at least in isolation, can achieve the open conformation in the absence of phosphorylation, although in solution this conformation is probably short-lived relative to a Tyr 393-phosphorylated state due to the lack of ionic stabilizing interactions between pTyr 393 and Arg 386.

Differences between the recognition of STI-571 and PD173955 by Abl kinase. Although STI-571 and PD173955 bind to essentially the same site in Abl kinase, the difference in size of the compounds imparts differences in binding characteristics. Interestingly, a total of 21 protein residues interact with STI-571 compared to only 11 residues with PD173955 (FIG. 4), yet PD173955 is found to be significantly more inhibitory. Thus, the extent of the binding interface (125I Å² for STI-571, 913 Å² for PD173955), which is often correlated with binding energy, cannot explain the greater potency of PD173955.

To reconcile this, we modeled PD173955 and STI-571 into both the open and closed conformations of Abl. The model of PD173955 in the structure of the inactive conformation of Abl reveals that it can nestle into the binding pocket without any major clashes with protein atoms (FIG. 7A), although minor adjustments of the P-loop are necessary. This loop is known to be highly flexible in kinases due to its high glycine content. As suggested in the AblK:STI-571 (variant) structure, the conformation of the P-loop is likely induced by the presence of the inhibitor. In this case, a small shift in the position of Tyr 253 is required in order to accommodate the additional sulphur-containing phenyl group of PD173955, which faces solvent.

In marked contrast, STI-571 cannot be accommodated in the form of Abl that PD173955 recognizes (FIG. 7B). Beyond the secondary amino group of STI-571, both the phenyl ring and piperazinyl ring collide with residues of the activation loop in the open conformation, most notably Asp 381 and Leu 384. Thus, PD173955 can probably bind to Abl regardless of the conformation of the activation loop, whereas STI-571 requires that the activation loop be in the closed conformation. Once STI-571 is bound to Abl, it jams between the activation loop and helix αC, preventing the activation loop from changing conformations. In the smaller PD173955 compound, the smaller size of the compound prevents the activation loop from being jammed. The fact that only the open conformation of the activation loop is observed in the structure of Ablk:PD173955 is perhaps a consequence of the requirements for crystal lattice formation. In solution, the isolated kinase domain of Abl probably exists in dynamic equilibrium between the open and closed conformations of the activation loop.

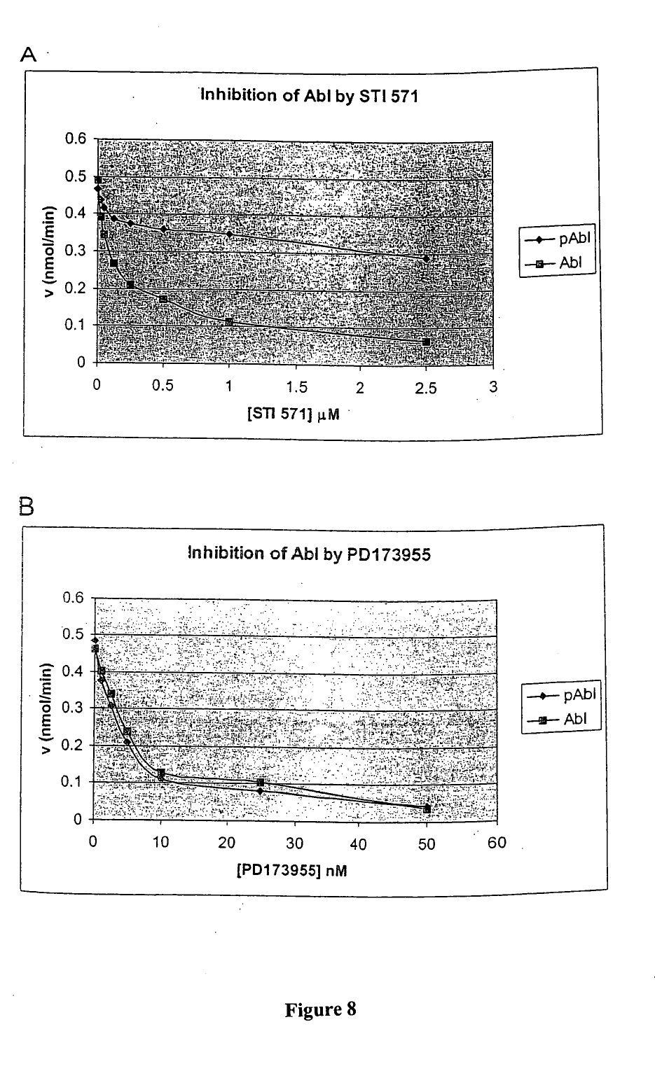

To test whether PD173955 is sensitive to activation loop phosphorylation, we performed a kinase inhibition assay with varying concentrations of PD173955 and STI-571 on both phosphorylated and unphosphorylated forms of the kinase domain of Abl. We have previously shown that STI-571 is an effective inhibitor only when the kinase is unphosphorylated (Schindler et al. Science 289:1938-1942, 2000; incorporated herein by reference). Based on the above modelling, we expect PD173955 to be insensitive to the phosphorylation state of Abl. Our results indicate that this is indeed the case. In the presence of STI-571, phosphorylated Abl displays significant levels of kinase activity (i.e. STI-571 is not inhibiting). Only upon dephosphorylation of Abl does STI-571 display significant inhibitory action (FIG. 8A), as previously seen (Schindler et al. Science 289:1938-1942, 2000; incorporated herein by reference). In contrast, the inhibition profile of PD173955 shows that the drug is effective against Abl regardless of its phosphophorylation state (FIG. 8B). Furthermore, based on the inhibition curves, it was found that PD173955 binds much more tightly than STI-571, inhibiting with an IC₅₀ of about 5 nM (independent of the phosphorylation state), while STI-571 inhibits at 100 nM (dephosphorylated Abl only). This suggests that although PD173955 makes fewer contacts with Abl, it inhibits more potently because it can recognize multiple forms of the kinase. STI-571 on the other hand, requires a specific conformation of the kinase before it can bind. If it were possible to isolate the Abl kinase domain in only the closed conformation, one might expect that the affinity of STI-571 for such a “frozen” Abl would likely surpass the affinity of PD173955 for Abl, since STI-571 interacts so much more extensively with the kinase.

Phosphorylation switch of the Abl kinase domain. In CML cells, c-Abl is fused with Bcr causing it to be constitutively active. Thus, the activation loop in Bcr-Abl would, for the most part, be phosphorylated and in the open conformation. Given that STI-571 cannot recognize this open conformation, how then does STI-571 achieve its great inhibitory effect? One possibility is that the phosphorylation state of the activation loop is dynamic. The action of cellular phosphatases constantly counteracts the kinase activity of the Bcr-Abl complex, and when the activation loop is transiently dephosphorylated, STI-571 can bind and inactivate the kinase. Another possibility is that STI-571 traps newly synthesized Bcr-Abl and shuts off its phosphorylation capability early on. PD173955, on the other, can probably inhibit Abl regardless of the phosphorylation state it is in.

It is hoped that by understanding how small molecule inhibitors such as STI-571 and PD173955 exert their influence at the molecular level, we can gain insight into making rational modifications to them in the hopes of producing drugs for improved leukemia therapy.

Current Studies to Identify New Potent Inhibitors of Bcr-Abl Kinase. We have been trying to develop an even better inhibitor of Bcr-Abl kinase than ST1571. Because PD173955 is quite insoluble, we have made various modifications of the compound to increase its solubility and hopefully also its potency and specificity. The new compounds are compared with PD173955 for their inhibitory properties. One compound, PD166326, was found to be ˜4-fold more inhibitory to Bcr-Abl-expressing cell lines than PD173955, and, as noted above, we are now mainly using PD16 in our current studies. Meanwhile, we are currently designing and synthesizing additional derivatives to attempt to further improve specificity and potency.

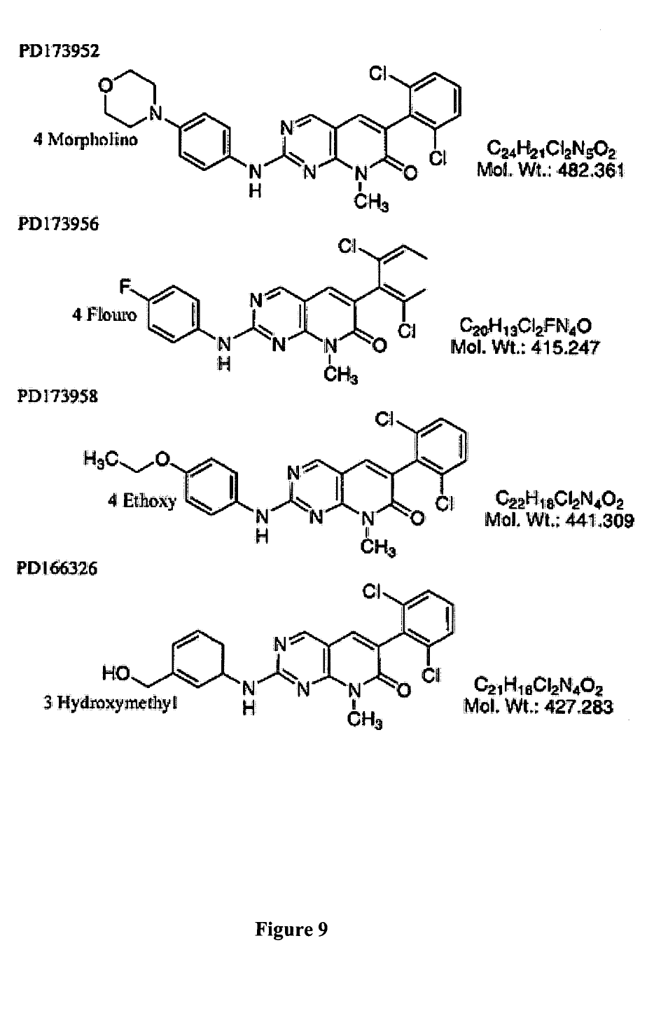

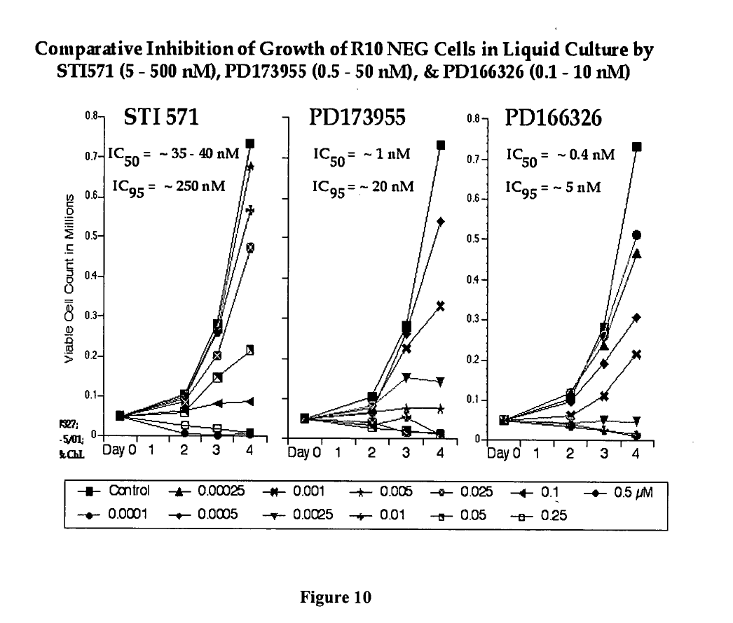

The structures of 4 of the analogues including, PD166326, is shown in FIG. 9 and their relative inhibitory activities for R10 Negative cells are shown in FIG. 10.

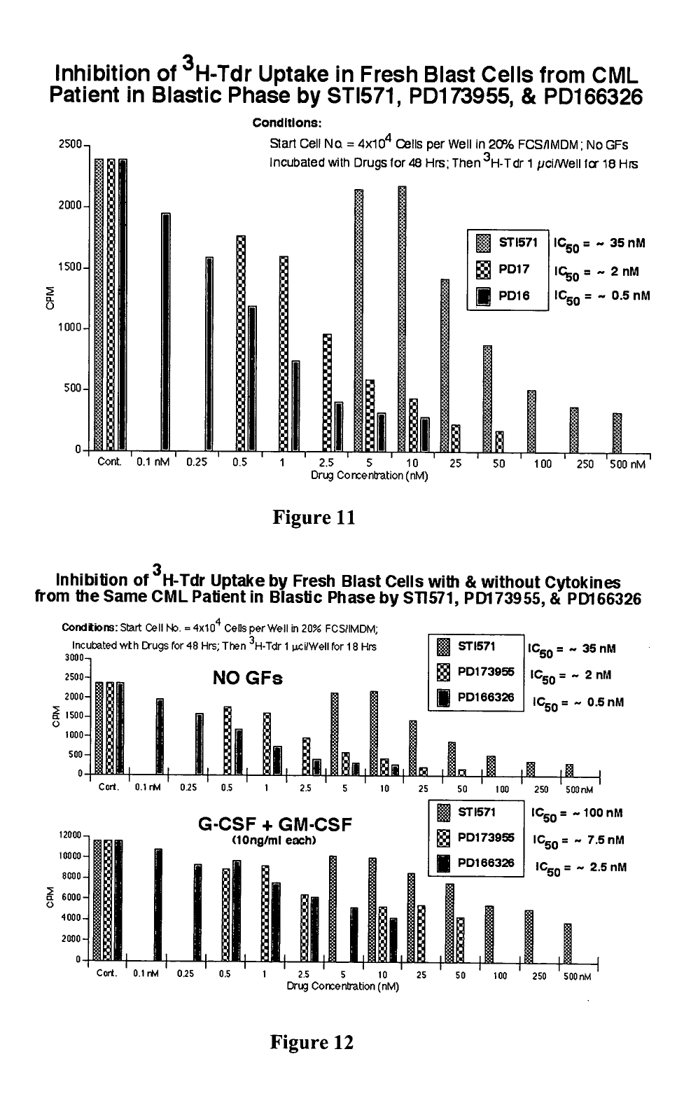

When comparing inhibitory activities of ST1571, PD17, and PD16 vs the R10-Negative subclone of M07/p210^{bcr-abl}; the comparative inhibitory results with the R10-Positive subclone are very similar. The estimated ratios of inhibition of c-kit (M07 cells growing in KL) to Bcr-Abl (R10-Negative cells) suggest that although PD17 and PD16 are 2.5- and 8-fold respectively more inhibitory to c-kit than ST1571, they are relatively more selective inhibitors of Bcr-Abl (ratios 25:1 and 40:1 for PD17 and PD16 respectively vs 3:1 for ST1571). As noted earlier and illustrated in FIGS. 11-12, PD16 is also 3-4-fold more inhibitory than PD17 to primary CML progenitor cells.

Because of the observations that ST1571 has shown significant therapeutic activity in GIST tumors (gastrointestinal stromal tumors) and some activity in SCL (small cell lung) cancer cells, and because PD173955 is even more inhibitory to c-kit and PDGF-R than ST1571, we have compared the activities of these inhibitors in several other human tumor cell lines which are known to have activating mutations, autocrine loops, or overexpression involving c-kit or PDGFR. We are currently trying to acquire one or more GIST cell lines from another institution (ie Dana Farber through Tyler Jacks) as none is available at MSKCC.

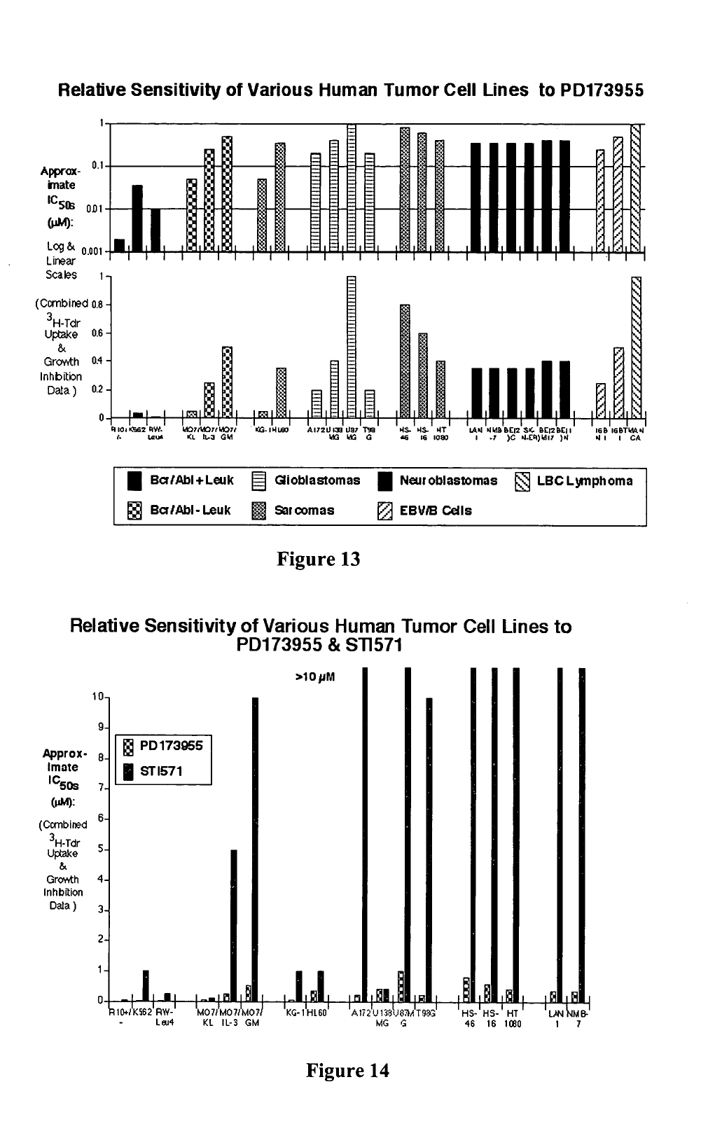

We have so far observed that PD173955 is considerably more inhibitory than ST1571 to 4 human glioblastoma cells lines and 4 human sarcoma cell lines, and we have found in our laboratory that PD173955 has similar inhibitory activities against 6 human neuroblastoma lines and 6 Ewing's sarcoma lines. The average IC₅₀s of PD173955 in these non-Bcr-Abl expressing cell lines ranged from ˜400 nM to 1 uM as compared to ˜2 nM for the Bcr-Abl-expressing R10 Negative and R10 Positive cell lines (ie 200-fold or less inhibitory). In FIG. 13, we have summarized some of the data on inhibition of the other human tumor cell lines by PD173955, including comparative experiments with ST1571 (FIG. 14). We have less data on PD16, but in the same 4 glioblastoma cell lines, in 3 of them PD16 was slightly less inhibitory than PD17.

Future Studies on Design and Synthesis of Inhibitors. Unlike STI571, which only binds to the inactive form of Abl, both PD173955 and PD166326 do not discriminate between the active and inactive forms of Abl. Co-crystallization studies have shown that while ST1571 has 6 hydrogen bonds, 21 van der Waals interactions with residues in the ATP binding pocket, and a significantly greater binding interface than PD173955, the latter has only 11 van der Waals interactions and forms only 2 hydrogen bonds. Thus while this might suggest that ST1571 may bind more tightly than PD17 in the inactive conformation of Abl, other considerations favor PD17's binding. In solution the isolated kinase domain of Abl probably exists in dynamic equilibrium between the open and closed conformations of the activation loop. The crystal structural studies suggest that PD17 inhibits Abl regardless of its phosphorylation state whereas ST1571 only binds when the kinase is unphosphorylated and this was confirmed experimentally. Based on the inhibition curves, PD17 inhibits with an IC₅₀ of ˜5 nM independent of the phosphorylation state while the IC50 for ST1571 is ˜100 nM for the dephosphorylated form only with no effect on the phosphorylated form.

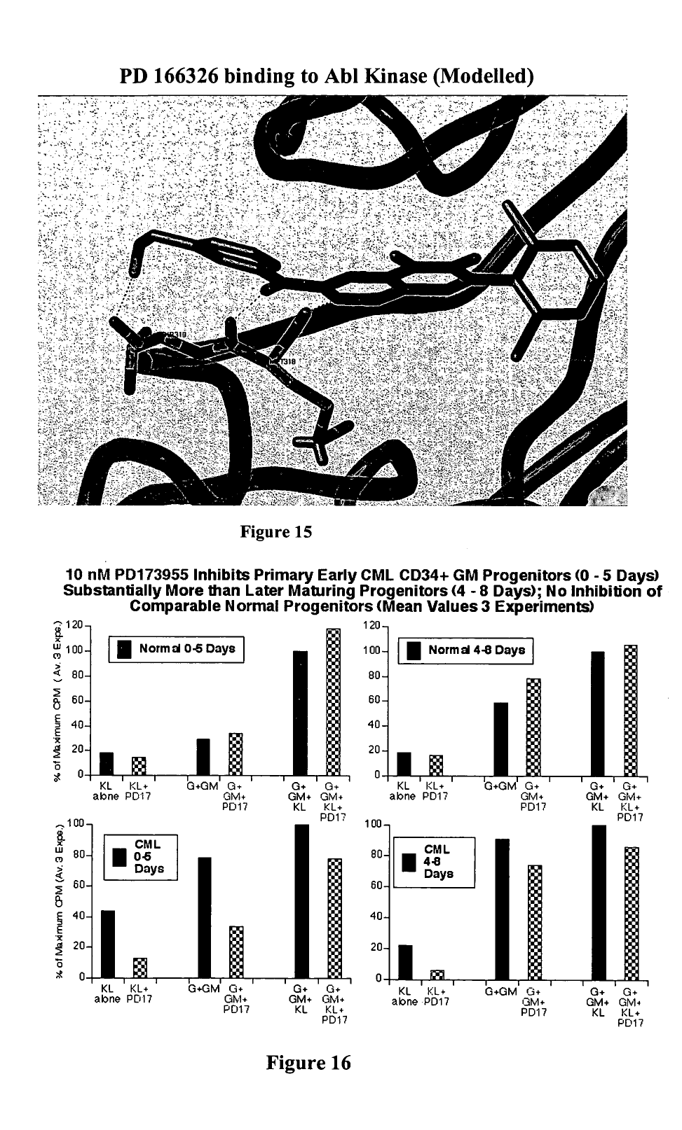

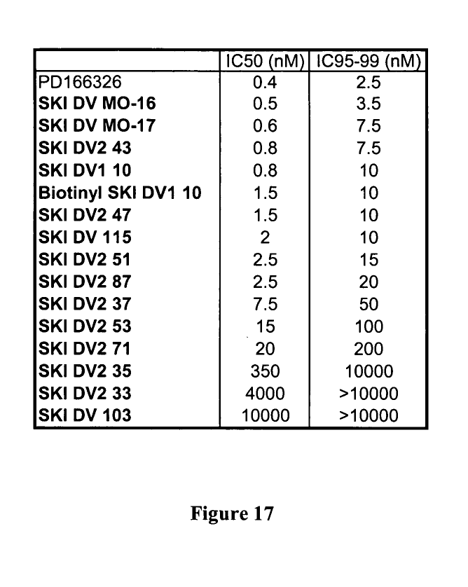

Of the pyridopyrimidine compounds so far synthesized PD166326 is the most inhibitory, probably because based on modeling of PD16 exchanged for PD17, it appears that the hydroxymethyl group on the phenyl ring forms an extra hydrogen bond not present with PD17 (FIG. 15).

We are using the structural data to model, design, and synthesize additional modified molecules with improved binding, solubility, and other desirable physical properties. Models have shown that the Thr³¹⁵→Ile³¹⁵ substitution as a result of the C→T mutation described by Sawyer's group [Gorre 2001] would result in a steric clash between PD17 and the methyl group of Ile³¹⁵ (as is also true of ST1571) which would probably prevent binding even though it has no hydrogen bond with Thr³¹⁵ as does ST1571.

We have recently synthesized several analogs that may circumvent this clash, but it must be stressed that this one C→T mutation is only one aspect of the overall problem of resistance. Sawyers' group and other investigators [von Bubnoff 2002.] have subsequently reported many other point mutations in the ATP binding pocket that also interfere with inhibitor binding and cause resistance. At least a dozen mutations have been reported in various locations and around the ATP pocket, some of which were predicted to confer much more resistance to ST1571 than others. We are now modeling the effects of these mutants on predicted resistance to PD16. As previously noted, several other important but unrelated mechanisms of resistance to ST1571 have also been reported, including resistance of quiescent CML stem cells.

We are also currently conducting or planning studies on the activities of PD16 in a variety of additional human tumor cell lines and animal models. We are also conducting or planning toxicological and pharmacological studies in mice and dogs, including determining the maximum tolerated doses, determining plasma and tissue levels and bioavailability after oral and parenteral administration, and developing optimal formulations for both oral and parenteral use. We will continue to evaluate the activities of PD16 in two CML murine model systems, the efficacy of ST1571 and PD16 in treating human mesothelioma xenografted tumors in mice, and compare PD16 and ST1571 in his retroviral transduction and transplantation murine CML model system.

From the knowledge gained from structural analysis and modeling studies we are optimistic that it will be possible to design and synthesize even more specific and potent inhibitors that also have improved solubility and other favorable physical characteristics that will be suitable for clinical use.

Differences in Response of Normal and CML Progenitors Committed to Different Lineages to Specific Cytokines and Inhibitors. Our recent studies comparing subpopulations of early and later normal and CML progenitors have shown that early CML GM progenitors are much more responsive to some single early-acting growth factors (e.g., KL, G-CSF, GM-CSF, EPO) than comparable normal progenitors. CML GM progenitors are also more responsive to low concentrations of G- + GM-CSF (0.03 ηg/ml of each) than normal progenitors; the former have an average 63% of the response to maximally stimulating concentrations of G + GM-CSF (10 ηg/ml) whereas normal GM progenitors only have 35%. The early CML progenitors are especially sensitive to low doses of the inhibitors. For example, 10 nM of PD17 caused an average inhibition of CML GM progenitors of 83% in KL alone at 50-100 ng/ml, 68% in G- + GM-CSF at 0.03 ng/ml, 61% in G- + GM-CSF at 10 ng/ml, and 34% in G- + GM + KL whereas at this dose of PD173955 (10 nM) there is no inhibition of normal progenitors with any of these cytokines singly or in combination (FIG. 16).

PD17 is on average about 20-fold more inhibitory to Bcr-Abl expressing cells than STI 571. (IC₅₀s=˜2 nM and 40 nM respectively for PD17 and ST1571 for R10 Negative and R10 Positive cells). Whereas the IC₅₀ of PD17 for CML progenitors varies in different patients between 1.5 and 7.5 nM, there is no detectable effect on normal progenitors at concentrations below 50 nM. As noted, the IC₅₀ of PD17 for normal progenitors is 350 nM. Inmost experiments with primary progenitors we used 10 nM of PD17 because it is well below the concentration inhibitory to normal progenitors.

As with the GM progenitors, the CML erythroid progenitors are much more sensitive to inhibition by PD17 than the normal progenitors. With 10 nM of PD17, normal erythroid progenitors are unaffected while the CML progenitors' exaggerated response to EPO alone or KL alone is almost completely blocked. Again, as with GM progenitors, KL partially protects CML erythroid progenitors from inhibition by PD17, and PD17 partially restores the normal synergistic response to EPO + KL.

We have less experimental data concerning the effects of PD166326 on primary progenitors, but, as in the case of the Bcr-Abl expressing cell lines, the comparative studies so far have shown it is ˜3-4-fold more inhibitory than PD17 to primary CML progenitors. Illustrative studies comparing PD17 and PD16 in inhibiting CD34+cells from a chronic phase patient and another in primary blasts from a patient in blastic phase are shown in FIGS. 11 & 12. FIG. 12 again also clearly illustrates the protective effect of some other cytokines besides KL, (i.e., here G-, and GM-CSF) against the inhibitors; the IC₅₀ is 3-5× higher with all 3 inhibitors in the presence of these cytokines than in their absence.

The studies briefly described above provide direct evidence that signaling pathways constitutively activated by Bcr-Abl kinase in primary primitive CML progenitors cooperates with signal transduction pathways activated by early-acting single cytokines (eg KL, G- and GM-CSF, EPO) to partially alleviate the normal requirement for the synergistic interaction of multiple cytokines in causing their activation. The receptors for these cytokines are present and fully operative on primitive CML progenitors, but it appears that their heightened response to single cytokines is not due to increased sensitivity of the receptors per se, but rather perhaps to Bcr-Abl constitutively altering the threshold or duration of activation of interactive downstream cytokines receptor signaling pathways, eg RAS, STAT5, PI-3 kinase—all of which have been implicated in Bcr-Abl mediated transformation.

### Example 2

### Synthesis of Pyrido-Pyrimidines

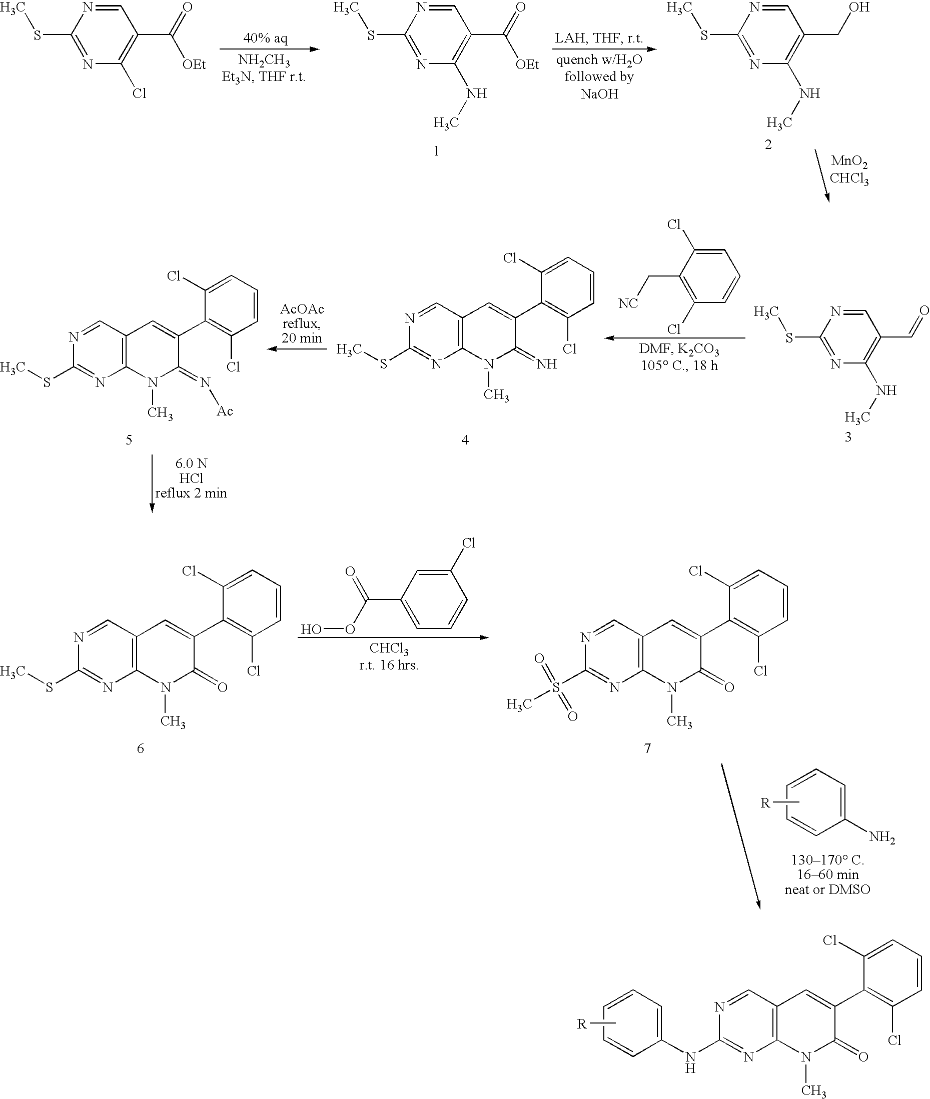 4-methylamino-2-methylsulfanyl-5-pyrimidinecarboxylate Ethyl Ester, 1.

To a solution of 50.261 g (0.216 mol) of commercially available 4-chloro-2-methylsulfanyl-5-pyrimidinecarboxylate ethyl ester in 600 mL of THF was added 91 mL (0.244 mol) of triethylamine followed by 93 mL of 40% aqueous methylamine. The solution warmed up slightly and was stirred for 1 hour. The THF was evaporated and the remaining aqueous slurry of white solid was partitioned between 300 mL of CHCl₃ and 200 mL of saturated aqueous 1NaHCO₃. The organic layer was washed with 200 mL of brine and dried over MgSO₄. The drying agent was removed by filtration and the CHCl₃ was concentrated to find a white solid. The solid was triturated in hexane, collected and dried under high vacuum to provide 43.71 g (89%) of 4-methylamino-2-methylsulfanyl-5-pyrimidinecarboxylate ethyl ester. The spectral data matched literature values.

(4-Methylamino-2-methylsulfanyl-pyrimidin-5-yl)-methanol, 2.

Lithium aluminum hydride (11.48 g, 302 mmol) was suspended in 600 mL of dry THF in a 2 L three-necked flask equipped with an overhead stirrer. The flask was purged with argon. In a separate 250 mL round-bottomed flask, 43.71 g (0.192 mol) of 4-methylamino-2-methylsulfanyl-5-pyrimidinecarboxylate ethyl ester was dissolved in 200 mL of dry THF. The solution of pyrimidine was transferred by cannula into a 250 mL addition funnel. The pyrimidine was added dropwise at room temperature over 30 minutes. The reaction was stirred for 1 hour and then quenched by carefully adding 25 mL of water dropwise over 30 minutes. Then, 25 mL of 15% w/v aqueous NaOH was added and lastly, another 75 mL portion of water was added. The greenish color faded and formed a pale green slurry which was stirred one additional hour. The precipitated aluminum salts were removed by vacuum filtration and the solids were washed well with EtOAc. The filtrate was evaporated to find a pale yellow slurry. This was suspended in 250 mL of 25% EtOAc/hexane. A colorless solid was isolated by vacuum filtration. After vacuum drying, 31.9 g (90%) of (4-methylamino-2-methylsulfanyl-pyrimidin-5-yl)-methanol was obtained. The spectral data matched literature values.

4-Methylamino-2-methylsulfanyl-pyrimidine-5-carbaldehyde, 3.

In a dry 3 L, three-necked round-bottomed flask fitted with an overhead mechanical stirrer and an argon inlet, 32.74 g (176.7 mmol) of (4-methylamino-2-methylsulfanyl-pyrimidin-5-yl)-methanol was dissolved in 1.6 L of CHCl₃. Manganese dioxide (152 g, 1.75 mol) was added portionwise with stirring. The reaction warmed slightly and was stirred for 7 hours. The manganese dioxide was removed by vacuum filtration through a pad of Celite and was washed well with two 300 mL portions of chloroform. The filtrate was concentrated to find a white solid. After vacuum drying, 30.6 g (94%) of 4-methylamino-2-methylsulfanyl-pyrimidine-5-carbaldehyde was isolated. The spectral data matched literature values.

6-(2,6-Dichlorophenyl)-8-methyl-2-methylsulfanyl-8H-pyrido[2,3-d]pyrimidin-7-ylideneamine, 4.

4-Methylamino-2-methylsulfanyl-pyrimidine-5-carbaldehyde (22.00 g, 120.1 mmol) and 2,6-dichlorophenylacetonitrile (26.90 g, 144.6 mmol) were added to a dry 1 L three-necked flask equipped with an overhead stirrer. The solids were dissolved in 125 mL of dry DMF. The pale yellow solution turned slightly orange upon addition of 70 g (506 mmol) of K₂CO₃. The reaction was heated to 100° C. and stirred for 18 hours at this temperature. The reaction became dark reddish-orange in color. The flask was cooled to 50° C. and the resulting precipitate was filtered. The pink solid was washed with 100 mL of 3:1 DMF/water. After vacuum drying, 13.45 g (32%) of 6-(2,6-dichloro-phenyl)-8-methyl-2-methylsulfanyl-8H-pyrido[2,3-d]pyrimidin-7-ylideneamine were obtained. The spectral data matched literature values.

6-(2,6-Dichlorophenyl)-8-methyl-2-methylsulfanyl-8H-pyrido[2,3-d]pyrimidin-7-one, 6.

6-(2,6-Dichlorophenyl)-8-methyl-2-methylsulfanyl-8H-pyrido[2,3-d]pyrimidin-7-ylideneamine (11.00 g, 31.3 mmol) was suspended in 75 mL of acetic anhydride with stirring under argon in a 250 mL round-bottomed flask fitted with a reflux condenser. The reaction was heated to reflux for 5 minutes and allowed to cool to room temperature. A bright yellow precipitate formed and the reaction was cooled further in an ice bath for 10 minutes. The acetylated product was filtered under vacuum and the cake rinsed well with 25 mL of acetic anhydride and 25 mL of diethyl ether. N-[6-(2,6-Dichlorophenyl)-8-methyl-2-methylsulfanyl-8H-pyrido[2,3-d]pyrimidin-7-ylidene]-acetamide, 5, was dried on the funnel and placed in a 250 mL round-bottomed flask. 6.0 N HCl (100 mL) was added to the acetylimine. This yellow suspension was heated rapidly to reflux, at which point, the yellow color paled significantly and more solid seemed to precipitate. Two minutes after reflux, the reaction was cooled to room temperature, then chilled further in an ice bath for 10 minutes. The solid was isolated by vacuum filtration and was washed with four 40 mL portions water, 40 mL of 2-propanol and 40 mL of diethyl ether. The faintly yellow-green powder was dried under vacuum. 6-(2,6-Dichlorophenyl)-8-methyl-2-methylsulfanyl-8H-pyrido[2,3-d]pyrimidin-7-one was isolated in 91% (9.96 g) yield. The spectral data matched literature values.

6-(2,6-Dichlorophenyl)-2-methanesulfonyl-8-methyl-8H-pyrido[2,3-d]pyrimidin-7-one, 7.

6-(2,6-Dichlorophenyl)-8-methyl-2-methylsulfanyl-8H-pyrido[2,3-d]pyrimidin-7-one (3.97 g, 11.3 mmol) was added to 300 mL of dry CHCl₃ in a 500 mL round-bottomed flask fitted with a mechanical stirrer. 3-Chloroperoxybenzoic acid (8.84 g, 28.2 mmol, assumed 55% w/w) was added slowly portionwise to the reaction. The reaction warmed 15-20° C. and was stirred for 2 hours. An additional portion (1.77 g, 6 mmol) was added and the reaction was stirred an additional hour. The reaction was poured into a separatory funnel and the organic layer washed with 200 mL saturated aqueous NaHCO₃. The organic layer was washed two times more with 100 mL of NaHCO₃, two times with 100 mL portions of water and once with 150 mL of saturated aqueous NaCl. The organic layer was dried over Na₂SO₄ for two hours. The drying agent was removed by filtration and the chloroform removed to find a pale-green solid. The 6-(2,6-dichlorophenyl)-2-methanesulfonyl-8-methyl-8H-pyrido[2,3-d]pyrimidin-7-one was triturated with CHCl₃ and then diethyl ether and dried under vacuum to find 4.30 g (99%) of the pale green powder. The spectral data matched literature values.

### General Methods for Displacement Reaction of Sulfone 7

Method A

6-(2,6-Dichloro-phenyl)-2-methanesulfonyl-8-methyl-8H-pyrido[2,3-d]pyrimidin-7-one and aniline derivative (3-4 molar equivalents) were combined in a flame-dried 10 mL flask. The mixture was mixed thoroughly. The flask was purged well with three vacuum/argon cycles. The flask was placed in a heated bath equilibrated at 170° C. for 5-30 minutes. The reaction was removed and allowed to cool for 5 minutes, and then 5 mL of ethyl acetate was added. This solution was allowed to stir for 30 minutes, over which time a precipitate formed. The solid was isolated by vacuum filtration and washed with a 2 mL of ethyl acetate and a 2 mL of diethyl ether. The crude product was purified by gradient silica gel column chromatography (0-15% MeOH in CH₂Cl₂).

Method B—Used when the product did not precipitate from ethyl acetate as in method A 6-(2,6-Dichloro-phenyl)-2-methanesulfonyl-8-methyl-8H-pyrido[2,3-d]pyrimidin-7-one and aniline derivative (3-4 molar equivalents) were combined in a flame-dried 10 mL flask. The mixture was mixed thoroughly. The flask was purged well with three vacuum/argon cycles. The flask was placed in a heated bath equilibrated at 170° C. for 5-30 minutes. The reaction was removed and allowed to cool for 5 minutes, and then 5 mL of ethyl acetate and 1 mL of methanol was added to dissolve any residual solids. The solution was poured into a separatory funnel containing 100 mL of ethyl acetate. The solution was washed 3 times with 50 mL of 1N HCl, 50 mL of water, 50 mL of saturated sodium bicarbonate and finally with 50 mL of saturated brine. The organic layer was dried over MgSO₄ for 15 min. The drying agent was removed by vacuum filtration and the solvent removed by rotary evaporation. The crude product was purified by gradient silica gel column chromatography (0-15% MeOH in CH₂Cl₂).

| | Method | | | |
|---|---|---|---|---|
| | of | | | |
| | Sulfone | | | |
| | 7 | | | |
| | Dis- | | | |
| | place- | | | |
| Aniline Derivative | ment | Pyridopyrimidine | | Yield |
| 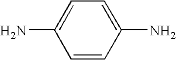 | Method A | SKI DV1- 10 | 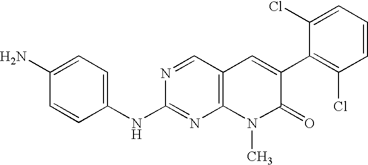 | 79% |
| 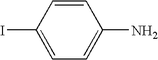 | Method A | SKI DV2- 37 | 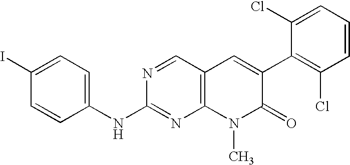 | 19% |
| 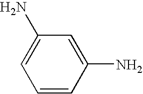 | Method A | SKI DV2- 43 | 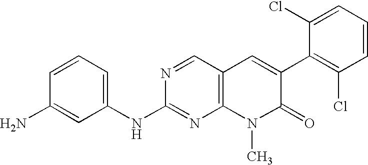 | 56% |
| 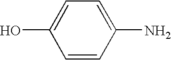 | Method A | SKI DV2- 47 | 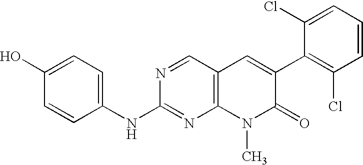 | 25% |
| 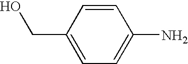 | Method B | SKI DV2- 51 | 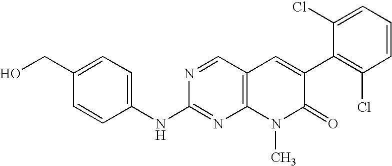 | 13% |
| 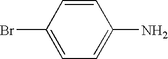 | Method A | SKI DV2- 53 | 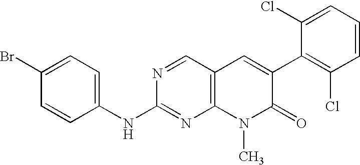 | 69% |
| 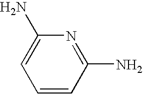 | Method A | SKI DV2- 71 | 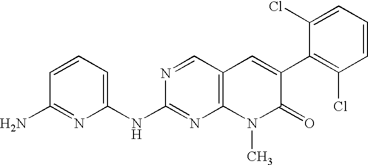 | 40% |
| 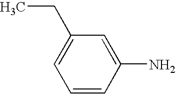 | Method A | SKI DV2- 87 | 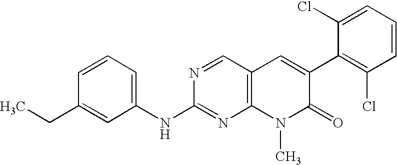 | 64% |
| 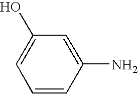 | Method A | SKI DV2- 89 | 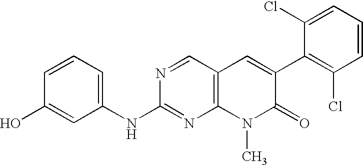 | 21% |
| 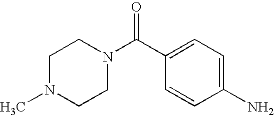 | Method B | SKI DV2- 131 | 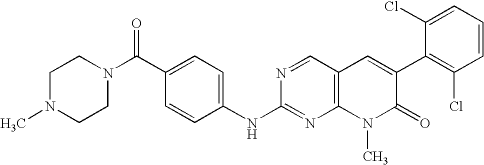 | 7% |

2-(3-Aminophenylamino)-6-(2,6-dichlorophenyl)-8-methyl-8H-pyrido[2,3-d]pyrimidin-7-one, SKI DV2-43 (50 mg) was placed in a 5 mL conical microwave reaction tube with stirrer. Acetic anhydride, 1.0 mL, was added to the tube and it was capped. The tube was irradiated for 180 s at 150° C. in a Smith Synthesizer® microwave reactor. The white solid was isolated by vacuum filtration and washed twice with 1 mL of isopropanol and twice with 1 mL of diethyl ether. The product was pure by standard analytical techniques. 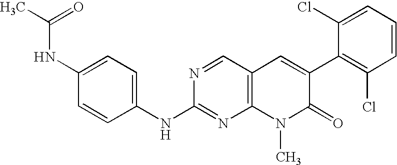
2-[4-(Acetamido)phenylamino]-6-(2,6-dichlorophenyl)-8-methyl-8H-pyrido[2,3-d]pyrimidin-7-one, SKI DV-M017

2-(4-Aminophenylamino)-6-(2,6-dichlorophenyl)-8-methyl-8H-pyrido [2,3-d]pyrimidin-7-one, SKI DV1-10 (50 mg, ×mmol) was placed in a 5 mL conical microwave reaction tube with stirrer. Acetic anhydride, 1.0 mL, was added to the tube and it was capped. The tube was irradiated for 180 s at 150° C. in a Smith Synthesizer® microwave reactor. The white solid was isolated by vacuum filtration and washed twice with 1 mL of isopropanol and twice with 1 mL of diethyl ether. The product was pure by standard analytical techniques. 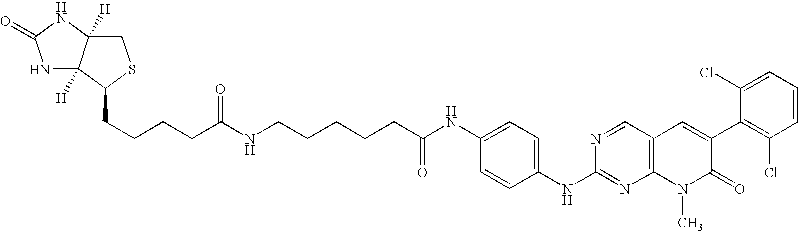
2-{4-[6-(Biotinamido)hexanamido]phenylamino}-6-(2,6-dichlorophenyl)-8-methyl-8H-pyrido[2,3-d]pyrimidin-7-one, SKI DV1-10 Biotinyl

2-(4-Aminophenylamino)-6-(2,6-dichlorophenyl)-8-methyl-8H-pyrido[2,3-d]pyrimidin-7-one, SKI DV1-10, (55 mg, 0.133 mmol), N-(+)-biotinyl-6-aminocaproic acid (57.2 mg, 0.160 mmol), 1-[3-(dimethylamino)propyl]-3-ethylcarbodiimide hydrochloride (123 mg, 0.64 mmol), and 4-dimethylaminopyridine (78 mg, 0.64 mmol) were combined in a 10 mL flame-dried round bottomed flask under argon. The solids were suspended in 5 mL of methylene chloride and a few drops of dimethylformamide. The solution was stirred for 12 hours. The reaction was poured into a separatory funnel containing 50 mL of methylene chloride. The organic layer was washed with three 50 mL portions of water and once with 50 mL of saturated brine. The organic layer was dried over magnesium sulfate, the drying agent removed by filtration and the solvent removed. The crude residue was purified by gradient silica gel column chromatography (0-15% 7M ammonia/methanol in methylene chloride). The product containing fractions were combined and evaporated. The product was pure by standard analytical techniques. 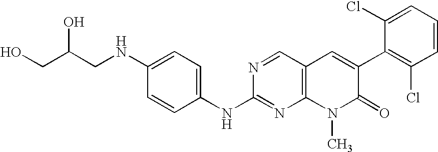
6-(2,6-Dichlorophenyl)-2-[4-(2,3-dihydroxypropylamino)-phenylamino]-8-methyl-8H-pyrido[2,3-d]pyrimidin-7-one, SKI DV2-33

2-(4-Aminophenylamino)-6-(2,6-dichlorophenyl)-8-methyl-8H-pyrido[2,3-d]pyrimidin-7-one, SKI DV1-10, (100 mg, 0.24 mmol) was dissolved in 1.0 mL of dry DMF. Cesium carbonate (240 mg, 0.73 mmol) was added in one portion. The yellow solution darkened to a golden color upon addition of base. Epichlorohydrin (40 mg, 38 uL, 0.49 mmol) was added by syringe. The reaction was stirred for 48 hours. The yellow color faded over the duration of the reaction. Approximately 1.0 mL of water was added to this and stirred for another 24 hours. The DMF and water was removed and the residue was take up in 5 mL of water, stirred vigorously and the precipitate isolated by filtration. The material was washed twice with 1 mL of water and twice with 1 mL of diethyl ether and dried under high vacuum to find 22 mg (19%) of a tan powder. 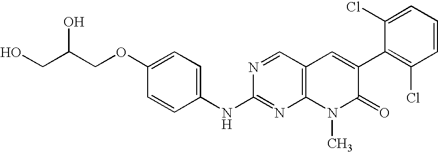
6-(2,6-Dichlorophenyl)-2-[3-(2,3-dihydroxypropoxymethyl)-phenylamino]-8-methyl-8H-pyrido[2,3-d]pyrimidin-7-one, SKI DV2-35

6-(2,6-Dichlorophenyl)-2-(3-hydroxymethylphenylamino)-8-methyl-8H-pyrido[2,3-d]pyrimidin-7-one, PD16326, (48 mg, 0.11 mmol) was dissolved in 1.0 mL of dry DMF. Cesium carbonate (110 mg, 0.34 mmol) was added in one portion. The yellow solution darkened to a golden color upon addition of base. Epichlorohydrin (20 mg, 18 uL, 0.22 mmol) was added by syringe. The reaction was stirred for 48 hours. The yellow color faded over the duration of the reaction. Approximately 1.0 mL of water was added to this and stirred for another 24 hours. The DMF and water was removed and the residue was take up in 5 mL of water, stirred vigorously and the precipitate isolated by filtration. The material was washed twice with 1 mL of water and twice with 1 mL of diethyl ether and dried under high vacuum to find 13 mg (25%) of an off-white powder. 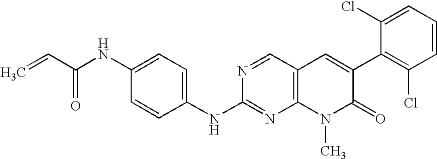
2-[4-(acrylamido)phenylamino]-6-(2,6-dichlorophenyl)-8-methyl-8H-pyrido[2,3-d]pyrimidin-7-one, SKI DV1-115

2-(4-Aminophenylamino)-6-(2,6-dichlorophenyl)-8-methyl-8H-pyrido[2,3-d]pyrimidin-7-one, SKI DV1-10, (41 mg, 0.099 mmol), acrylic acid (8.6 mg, 0.119 mmol), 1-[3-(dimethylamino)propyl]-3-ethylcarbodiimide hydrochloride (76 mg, 0.40 mmol), and diisopropylethylamine (0.070 mL, 0.40 mmol) were combined in a 10 mL flame-dried round bottomed flask under argon. The solids were suspended in 5 mL of methylene chloride and a few drops of dimethylformamide. The solution was stirred for 12 hours. The reaction was poured into a separatory funnel containing 50 mL of methylene chloride. The organic layer was washed with three 50 mL portions of water and once with 50 mL of saturated brine. The organic layer was dried over magnesium sulfate, the drying agent removed by filtration and the solvent removed. The crude residue was purified by gradient silica gel column chromatography (0-15% MeOH/CH₂Cl₂). The product containing fractions were combined and evaporated. The product was pure by standard analytical techniques.
Synthesis of Aniline Sidechain for SKI DV2-131 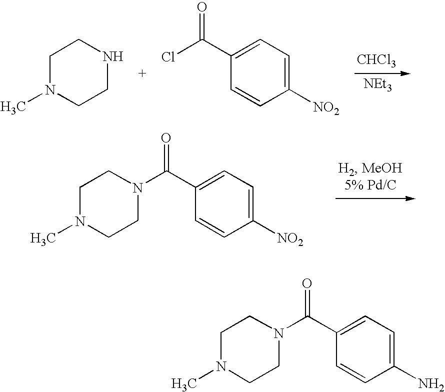
4-Methyl-1-(4-nitrobenzoyl)piperazine

1-Methylpiperazine (13.49 g, 135 mmol) and triethylamine (16.4 g, 162 mmol) were added to 200 mL of CHCl₃ in a 500 mL round-bottomed flask equipped with a 250 mL addition funnel under argon with a magnetic stirring bar. 4-nitrobenzoyl chloride (25.00 g, 135 mmol) was dissolved in 200 mL of CHCl₃ with slight warming. The solution of benzoyl chloride was placed in the addition funnel and was added slowly to the solution of piperazine. The solution warmed slightly and was allowed to stir 1 hour after the addition was complete. The reaction was concentrated until the product began to precipitate. The product was isolated by vacuum filtration and rinsed with two 25 mL portions of cold diethyl ether. After recrystallization from 95% ethanol, the product, 4-methyl-1-(4-nitrobenzoyl)piperazine, was pure by standard analytical techniques.

4-Methyl-1-(4-aminobenzoyl)piperazine

4-Methyl-1-(4-nitrobenzoyl)piperazine (5 g, 20.0 mmol) and 5% palladium on carbon (427 mg, 0.20 mmol) were suspended in 50 mL of dry methanol in a 200 mL threaded pressure bottle under argon with magnetic stirrer bar. The flask was vacuum/argon purged three times, vacuum/H₂ gas purged twice and charged with 40 psi of H₂ gas. The reaction warmed considerably and the H₂ absorption subsided after 45 minutes. The system was flushed with argon and the Pd/C removed by filtration over Celite. The methanol was removed to find a slightly yellow oil. This oil was suspended in dry diethyl ether and re-evaporated several times to remove traces of methanol. The product, 4-methyl-1-(4-aminobenzoyl)piperazine, was pure by standard analytical techniques. This aniline derivative was used directly for the synthesis of SKI DV2-131.

### Example 3

### Effects of Inhibitors in Animal Models of CML

The present treatment of chronic myelogenous leukemia (CML) is unsatisfactory and the majority of patients are still dying of the disease. Recent treatment protocols with chemotherapy and interferon have prolonged life by about a year, but more intensive treatment protocols have not resulted in significant further improvement. The only curative treatment is by intensive chemotherapy and/or irradiation followed by rescue with allogeneic bone marrow transplantation. Only a minority of patients with CML have suitable matched donors and are eligible for transplantation; elderly patients, many of whom have other diseases, are unable to tolerate the intensive therapy required to cure the disease. More effective and less toxic treatment are clearly needed.

CML is an excellent target for development of selective treatment because of its highly consistent genetic abnormality and qualitatively different fusion gene product with constitutive tyrosine kinase activity, p210^{bcr-abl}. The p210^{bcr-abl} fusion protein has been shown to have a key role in constitutively phosphorylating a large number of proteins involved in signaling pathways, thereby severely dysregulating a number of critical regulatory networks. The p210^{bcr-abl} protein appears to be solely responsible for all the initial manifestations of the chronic phase of this disease, and CML is thus an excellent model of an early form of human cancer due to a single acquired genetic abnormality.

Except for a minority of patients who have apparently been cured by marrow ablative therapy and bone marrow transplantation the majority of patients are still dying of their disease. The overall median survival of patients with Ph positive CML in chronic phase from diagnosis treated with conventional chemotherapy has varied from around 3-5 years in different series, with a range of less than a year to over 20 years. Survival after development of an accelerated phase is usually less than a year and after blastic transformation only a few months, although patients with lymphoblastic transformation may live longer with appropriate chemotherapy

Because it has been known for about 20 years that the increased protein tyrosine kinase (PTK) activity of the oncogenic Bcr-Abl fusion proteins is essential for transformation, many investigators have examined various PTK inhibitors, hoping to find one that will selectively inhibit Bcr-Abl kinase. One of the most potent and selective inhibitors of Bcr-Abl kinase activity so far discovered is the Novartis compound ST1571 (Gleevec) (formerly Ciba-Geigy compound CGP57148), and STI571 has recently been undergoing clinical trials in patients with CML. ST1571 acts as a competitive inhibitor of ATP at the ATP binding site of the tyrosine kinase domains of both the normal Abl and Bcr-Abl. However, molecular or cytogenetic causes of resistance to Gleevec, including various mutations in the ATP binding site of Bcr-Abl, increased expression of Bcr-Abl protein, amplification of the Bcr-Abl gene, and novel cytogenetic aberrations. ST1571 is also effective in inducing partial and sometimes complete hematologic and cytogenetic responses in accelerated and blastic phase disease, but the responses are generally less complete and of shorter duration, and resistance may develop rapidly.

Thus while Gleevec provides a very good example of how effective a selective, molecularly-targeted small molecule can be in producing a high incidence of remissions with relatively little toxicity, it is doubtful if any of the patients have been cured with this single agent. Moreover the majority of patients treated for long periods have already developed one or more mechanisms of resistance to the drug, leading to diminished responsiveness or relapse.

PD173955 is a member of a new class of highly potent tyrosine kinase inhibitors based on the pyrido[2,3-d]pyrimidine core template (Trumpp-Kallmeyer et al. J. Med. Chem. 41:1752-1763, 1998; incorporated herein by reference). PD17 is on average about 20-fold more inhibitory to CML progenitors than STI 571. The IC₅₀ of PD17 for CML progenitors is ˜2 nM, but there is no detectable affect on normal progenitors at concentrations below 50 nM. In most experiments with primary progenitors 10 nM of PD17 was used because it is well below the concentration inhibitory to normal progenitors. Another pyridopyrimidine compound synthesized more recently, PD166326, is ˜3-4-fold more inhibitory than PD17 to CML progenitors both in chronic phase and in blastic phase as well as in R10 Negative cells.

The CML erythroid progenitors are much more sensitive to inhibition by PD17 than the normal progenitors. With 10 ηM of PD17, normal erythroid progenitors are unaffected while the CML progenitors' exaggerated response to EPO alone or KL alone is almost completely blocked. Again, as with GM progenitors, KL partially protects CML erythroid progenitors from inhibition by PD17 and PD17 partially restore the normal synergistic response to EPO+KL.

Using the vital stain, CFSE, it is possible to determine the concentrations of different inhibitors required to arrest cell division and also to quantitate the number of quiescent cells. For example, some R10-Negative cells stained with CFSE make one division by Day 2 after exposure to 25 nM of PD17, but subsequent divisions are blocked. However with exposure to ST1571 at the same concentration many cells continue dividing and 80% are still viable after 6 days; even at 100 nM ST1571, some cells are able to continue dividing and the viability is still 24% at 6 days. Primitive quiescent early progenitors are relatively insensitive to ST1571, and suggest similar insensitivity of Go progenitors to PD16.

Co-crystallizing PD173955 with the Abl kinase domain revealed that PD173955 binds to a conformation in which the activation loop resembles that of an active kinase domain. Furthermore, modeling has shown that PD173955 can also be accommodated in the kinase domain when the activation loop is in the inactive conformation. In contrast, as noted previously, ST1571 is able to associate with Abl kinase only when the activation loop is in the inactive conformation. Abl kinase can assume both active and inactive conformations independent of phosphorylation of Tyr393, the major site of phosphorylation in the Abl activation loop. Although the conformational changes are very rapid and dynamic, Bcr presumably forces Abl to adopt mainly an active conformation, thereby favoring its association with PD173955 over ST1571.

Designing Optimal Abl Inhibitors and Exploring the Conformational Flexibility of Abl Kinase.

The compounds of the present invention are based on work using the structural data to model, design, and synthesize additional modified molecules with improved binding, solubility, and other desirable physical properties. Models have shown that the Thr³¹⁵→Ile³¹⁵ substitution as a result of the C→T mutation described by Sawyer's group (Gorre et al. Science 293:876-880, 2001; incorporated herein by reference) would result in a steric clash between PD17 and the methyl group of Ile³¹⁵ (as is also true of ST1571) which would probably prevent binding even though it has no hydrogen bond with Thr³¹⁵ as does ST1571. Some of the compounds of the present invention may circumvent this clash, but it must be stressed that this one C→T mutation is only one aspect of the overall problem of resistance. Sawyers and other investigators (Deininger et al. Cancer Research 61:8005-8013, 2001; Jena et al. 2002; Parada et al. J. Biol. Chem. 276:23572-23580, 2001; von Bubnoffet al. Lancet 359:487-491, 2002; each of which is incorporated herein by reference) have subsequently reported other point mutations in the ATP binding pocket that also interfere with inhibitor binding and cause resistance, and we have recently started to study the effects of multiple analogues on cells resistant to ST1571. As previously noted, several other unrelated important mechanisms of resistance to ST1571 have been reported including resistance of quiescent CML stem cells. Thus there are many other factors to be considered besides this one mutation in designing an optimally-specific Abl inhibitor.

PD173955 is quite insoluble; therefore, 21 modifications of the compound have been made to increase its solubility and hopefully also its potency and specificity. The inhibitory properties of these new analogues were compared with PD173955 and PD166326; and 4 others in addition to PD166326 are ˜4-fold more inhibitory to Bcr-Abl-expressing cell lines than PD173955 while others are much less inhibitory.

Effect of Inhibitors in Animal Models of CML

Toxicological and pharmacological studies in mice and dogs include determining the maximum tolerated doses, measuring plasma and tissue levels and bioavailability after oral and parenteral administration, and developing optimal formulations for both oral and parenteral use. It has already been found by measuring serial plasma levels in the mice that bioavailability of PD166326 after oral administration is only about 8% of that after intravenous or intraperitoneal injection. One way to administer the drug is by continuous infusion, and we are currently exploring the use of small osmotic pumps that can be inserted into the peritoneal cavities of mice that will deliver a constant concentration continuously. All animal studies are performed under the standard guidelines. Initial dose escalation studies are performed via oral delivery on control mice in order to determine the maximum tolerated dose (MTD). Studies proceed at or below half-MTD. The inherent kinetics for disease engraftment of each model system and the appropriate windows for duration of drug administration are then determined. In the human mesothelioma xenograft model, tumor formation is evaluated, allowing for the establishment of 1-2 mm³ tumors before drug administration. Survival data are presented using the Kaplan-Meyer method. We will compare the administration of the inhibitors by i.v. or i.p. bolus injection with continuous i.p. infusion using an osmotic pump. In order to investigate the effects of PD166326 or any subsequent analogues in vivo using a modified murine CML model initially described by Pear et al. (Blood 92:3780-3792, 1998; incorporated herein by reference), where murine bone marrow stem cells are infected with a retrovirus that expresses Bcr-Abl and then adoptively transferred into a lethally irradiated host (Wolff et al. Blood 98:2808-2816, 2001; incorporated herein by reference).

Primary normal and CML progenitor cells are also tested in assays to determine optimally effective therapeutic concentrations. The inhibitory concentrations on purified Abl kinase in vitro of all the analogues are measured in order to compare these with the inhibitory concentrations in cellular assays using Bcr-Abl-expressing cell lines. Such comparisons are essential in determining whether differences found in cellular assays are due to differences in pharmacological properties rather than inhibition of Bcr-Abl kinase per se.

### Example 4

### Inhibition of Wild-Type and Mutant Bcr-Abl by Pyrido-Pyrimidine-Type Small Molecule Kinase Inhibitors

Introduction

The discovery of the Philadelphia chromosome, a reciprocal translocation between the long arms of chromosomes 9 and 22 (Nowell, P. C. and Hungerford, D. A minute chromosome in human granulocytic leukaemia. Science, 132: 1497-1501, 1960; Rowley, J. D. Letter: A new consistent chromosomal abnormality in chronic myelogenous leukaemia identified by quinacrine fluorescence and Giemsa staining. Nature, 243: 290-293, 1973; each of which is incorporated herein by reference), is the characteristic molecular abnormality present in approximately 95% of cases of chronic myelogenous leukemia (CML) and in up to 20% of adult acute lymphoblastic leukemia (ALL) (Faderl, S., Talpaz, M., Estrov, Z., O'Brien, S., Kurzrock, R., and Kantarjian, H. M. The biology of chronic myeloid leukemia. N. Engl. J. Med., 341: 164-172, 1999; Sawyers, C. L. Chronic myeloid leukemia. N. Engl. J. Med., 340: 1330-1340, 1999; each of which is incorporated herein by reference). Bcr-Abl, the resulting fusion protein, is a deregulated, constitutively active oncogenic protein tyrosine kinase. When Bcr-Abl is introduced in mice, it causes CML (Daley, G. Q., Van Etten, R. A., and Baltimore, D. Induction of chronic myelogenous leukemia in mice by the P210bcr/abl gene of the Philadelphia chromosome. Science, 247: 824-830, 1990; incorporated herein by reference), and transformation is strictly dependent on its tyrosine kinase activity (Lugo, T. G., Pendergast, A. M., Muller, A. J., and Witte, 0. N. Tyrosine kinase activity and transformation potency of bcr-abl oncogene products. Science, 247: 1079-1082, 1990; incorporated herein by reference). Thus, Bcr-Abl constitutes an attractive target for molecular-based therapy of Ph+ ALL and CML.

A high throughput screen of compound libraries at Ciba-Geigy (now Novartis) identifified imatinib mesylate, a derivative of the initial 2-phenylaminopyrimidine lead compound, to inhibit several tyrosine kinases, among them the protein tyrosine kinase Abl (Zimmermann, J., Buchdunger, E., Mett, H., Meyer, T., and Lydon, N. B. Potent and selective inhibitors of the ABL-kinase: phenylaminopyrimidine (PAP) derivatives. Bioorg Med Chem Lett, 7:187-192, 1997; Buchdunger, E., Zimmermann, J., Mett, H., Meyer, T., Muller, M., Druker, B. J., and Lydon, N. B. Inhibition of the Abl protein-tyrosine kinase in vitro and in vivo by a 2-phenylaminopyrimidine derivative, Cancer Res., 56: 100-104, 1996; each of which is incorporated herein by reference). Imatinib was demonstrated to inhibit the corresponding oncogenic fusion protein Bcr-Abl p210 (Druker, B. J., Tamura, S., Buchdunger, E., Ohno, S., Segal, G. M., Fanning, S., Zimmermann, J., and Lydon, N. B. Effects of a selective inhibitor of the Abl tyrosine kinase on the growth of Bcr-Abl positive cells. Nat. Med., 2: 561-566, 1996; incorporated herein by reference), and p185 (Beran, M., Cao, X., Estrov, Z., Jeha, S., Jin, G., O'Brien, S., Talpaz, M., Arlinghaus, R. B., Lydon, N. B., and Kantarjian, H. Selective inhibition of cell proliferation and BCR-ABL phosphorylation in acute lymphoblastic leukemia cells expressing Mr 190,000 BCR-ABL protein by a tyrosine kinase inhibitor (CGP-57148). Clin. Cancer Res., 4: 1661-1672, 1998; incorporated herein by reference), and displayed activity in a murine model of Bcr-Abl-induced leukemia (Buchdunger, E., Zimmermann, J., Mett, H., Meyer, T., Muller, M., Druker, B. J., and Lydon, N. B. Inhibition of the Abl protein-tyrosine kinase in vitro and in vivo by a 2-phenylaminopyrimidine derivative, Cancer Res., 56: 100-104, 1996; le Coutre, P., Mologni, L., Cleris, L., Marchesi, E., Buchdunger, E., Giardini, R., Formelli, F., and Gambacorti-Passerini, C. In vivo eradication of human BCR/ABL-positive leukemia cells with an ABL kinase inhibitor, J. Natl. Cancer Inst., 91: 163-168, 1999; each of which is incorporated herein by reference). Impressive and encouraging response rates resulted from the conducted Phase I and II clinical studies determining the activity of imatinib in chronic phase CML (Druker, B. J., Talpaz, M., Resta, D. J., Peng, B., Buchdunger, E., Ford, J. M., Lydon, N. B., Kantarjian, H., Capdeville, R., Ohno-Jones, S., and Sawyers, C. L. Efficacy and safety of a specific inhibitor of the BCR-ABL tyrosine kinase in chronic myeloid leukemia. N. Engl. J. Med., 344: 1031-1037, 2001; Kantarjian, H., Sawyers, C., Hochhaus, A., Guilhot, F., Schiffer, C., Gambacorti-Passerini, C., Niederwieser, D., Resta, D., Capdeville, R., Zoellner, U., Talpaz, M., Druker, B., Goldman, J., O'Brien, S. G., Russell, N., Fischer, T., Ottmann, O., Cony-Makhoul, P., Facon, T., Stone, R., Miller, C., Tallman, M., Brown, R., Schuster, M., Loughran, T., Gratwohl, A., Mandelli, F., Saglio, G., Lazzarino, M., Russo, D., Baccarani, M., and Morra, E. Hematologic and cytogenetic responses to imatinib mesylate in chronic myelogenous leukemia. N. Engl. J. Med., 346: 645-652, 2002; each of which is incorporated herein by reference), but also in advanced-phase CML, such are accelerated phase and blast crisis CML, and Ph+ALL (Druker, B. J., Sawyers. C. L., Kantarjian, H., Resta, D. J., Reese, S. F., Ford, J. M., Capdeville, R., and Talpaz, M. Activity of a specific inhibitor of the BCR-ABL tyrosine kinase in the blast crisis of chronic myeloid leukemia and acute lymphoblastic leukemia with the Philadelphia chromosome. N. Engl. J. Med., 344: 1038-1042, 2001; Talpaz, M., Silver, R. T., Druker, B. J., Goldman, J. M., Gambacorti-Passerini, C., Guilhot, F., Schiffer, C. A., Fischer, T., Deininger, M. W., Lennard, A. L., Hochhaus, A., Ottmann, O. G., Gratwohl, A., Baccarani, M., Stone, R., Tura, S., Mahon, F. X., Fernandes-Reese, S., Gathmann, I., Capdeville, R., Kantarjian, H. M., and Sawyers, C. L. Imatinib induces durable hematologic and cytogenetic responses in patients with accelerated phase chronic myeloid leukemia: results of a phase 2 study. Blood, 99: 1928-1937, 2002; Sawyers, C. L., Hochhaus, A., Feldman, E., Goldman, J. M., Miller, C. B., Ottmann, 0. G., Schiffer, C. A., Talpaz, M., Guilhot, F., Deininger, M. W., Fischer, T., O'Brien, S. G., Stone, R. M., Gambacorti-Passerini, C. B., Russell, N. H., Reiffers, J. J., Shea, T. C., Chapuis, B., Coutre, S., Tura, S., Morra, E., Larson, R. A., Saven, A., Peschel, C., Gratwohl, A., Mandelli, F., Ben Am, M., Gathmann, I., Capdeville, R., Paquette, R. L., and Druker, B. J. Imatinib induces hematologic and cytogenetic responses in patients with chronic myelogenous leukemia in myeloid blast crisis: results of a phase II study. Blood, 99: 3530-3539, 2002; Ottmann, O. G., Druker, B. J., Sawyers, C. L., Goldman, J. M., Reiffers, J., Silver, R. T., Tura, S., Fischer, T., Deininger, M. W., Schiffer, C. A., Baccarani, M., Gratwohl, A., Hochhaus, A., Hoelzer, D., Fernandes-Reese, S., Gathmann, I., Capdeville, R., and O'Brien, S. G. A phase 2 study of imatinib in patients with relapsed or refractory Philadelphia chromosome-positive acute lymphoid leukemias. Blood, 100: 1965-1971, 2002; each of which is incorporated herein by reference).

However, it became clear that the majority of patients suffering from advanced phase CML and Ph+ ALL experience a relapse of their disease despite continued treatment with the drug (Druker, B. J., Sawyers, C. L., Kantarjian, H., Resta, D. J., Reese, S. F., Ford, J. M., Capdeville, R., and Talpaz, M. Activity of a specific inhibitor of the BCR-ABL tyrosine kinase in the blast crisis of chronic myeloid leukemia and acute lymphoblastic leukemia with the Philadelphia chromosome. N. Engl. J. Med., 344: 1038-1042, 2001; Sawyers, C. L., Hochhaus, A., Feldman, E., Goldman, J. M., Miller, C. B., Ottmann, O. G., Schiffer, C. A., Talpaz, M., Guilhot, F., Deininger, M. W., Fischer, T., O'Brien, S. G., Stone, R. M., Gambacorti-Passerini, C. B., Russell, N. H., Reiffers, J. J., Shea, T. C., Chapuis, B., Coutre, S., Tura, S., Morra, E., Larson, R. A., Saven, A., Peschel, C., Gratwohl, A., Mandelli, F., Ben Am, M., Gathmann, I., Capdeville, R., Paquette, R. L., and Druker, B. J. Imatinib induces hematologic and cytogenetic responses in patients with chronic myelogenous leukemia in myeloid blast crisis: results of a phase II study. Blood, 99: 3530-3539, 2002; Ottmann, O. G., Druker, B. J., Sawyers, C. L., Goldman, J. M., Reiffers, J., Silver, R. T., Tura, S., Fischer, T., Deininger, M. W., Schiffer, C. A., Baccarani, M., Gratwohl, A., Hochhaus, A., Hoelzer, D., Femandes-Reese, S., Gathmann, I., Capdeville, R., and O'Brien, S. G. A phase 2 study of imatinib in patients with relapsed or refractory Philadelphia chromosome-positive acute lymphoid leukemias. Blood, 100: 1965-1971, 2002; each of which is incorporated herein by reference). Kinase domain mutations that interfere with drug binding while retaining ATP binding and catalytic activity constitute the major cause of resistance (Gorre, M. E., Mohammed, M., Ellwood, K., Hsu, N., Paquette, R., Rao, P. N., and Sawyers, C. L. Clinical resistance to STI-571 cancer therapy caused by BCR-ABL gene mutation or amplification. Science, 293: 876-880, 2001; Branford, S., Rudzki, Z., Walsh, S., Grigg, A., Arthur, C., Taylor, K., Herrmann, R., Lynch, K. P., and Hughes, T. P. High frequency of point mutations clustered within the adenosine triphosphate-binding region of BCR/ABL in patients with chronic myeloid leukemia or Ph-positive acute lymphoblastic leukemia who develop imatinib (ST1571) resistance. Blood, 99: 3472-3475, 2002; von Bubnoff, N., Schneller, F., Peschel, C., and Duyster, J. BCR-ABL gene mutations in relation to clinical resistance of Philadelphia-chromosome-positive leukaemia to ST1571: a prospective study. Lancet, 359: 487-491, 2002; Hofmann, W. K., Jones, L. C., Lemp, N. A., de Vos, S., Gschaidmeier, H., Hoelzer, D., Ottmann, O. G., and Koeffler, H. P. Ph (+) acute lymphoblastic leukemia resistant to the tyrosine kinase inhibitor ST1571 has a unique BCR-ABL gene mutation. Blood, 99: 1860-1862, 2002; Roche-Lestienne, C., Soenen-Comu, V., Grardel-Duflos, N., Lai, J. L., Philippe, N., Facon, T., Fenaux, P., and Preudhomme, C. Several types of mutations of the Abl gene can be found in chronic myeloid leukemia patients resistant to ST1571, and they can pre-exist to the onset of treatment. Blood, 100: 1014-1018, 2002; Shah, N. P., Nicoll, J. M., Nagar, B., Gorre, M. E., Paquette, R. L., Kuriyan, J., and Sawyers, C. L. Multiple BCR-ABL kinase domain mutations confer polyclonal resistance to the tyrosine kinase inhibitor imatinib (ST1571) in chronic phase and blast crisis chronic myeloid leukemia. Cancer Cell, 2: 117-125, 2002; Hochhaus, A., Kreil, S., Corbin, A. S., La Rosee, P., Muller, M. C., Lahaye, T., Hanfstein, B., Schoch, C., Cross, N. C., Berger, U., Gschaidmeier, H., Druker, B. J., and Hehlmann, R. Molecular and chromosomal mechanisms of resistance to imatinib (ST1571) therapy. Leukemia, 16: 2190-2196, 2002; each of which is incorporated herein by reference), detected in at least 70% of patients with CML or Ph+ ALL relapsing after an initial hematologic response to imatinib (von Bubnoff, N., Peschel, C., and Duyster, J. Resistance of Philadelphia-chromosome positive leukemia towards the kinase inhibitor imatinib (ST1571, Glivec): a targeted oncoprotein strikes back. Leukemia, in press: 2003; incorporated herein by reference). The causality between mutations in BCR-ABL and refractoriness of Ph+leukemia towards imatinib was proved by introducing mutated BCR-ABL into cell lines, thereby conferring resistance to the drug (Gorre, M. E., Mohammed, M., Ellwood, K., Hsu, N., Paquette, R., Rao, P. N., and Sawyers, C. L. Clinical resistance to STI-571 cancer therapy caused by BCR-ABL gene mutation or amplification. Science, 293: 876-880, 2001; von Bubnoff, N., Schneller, F., Peschel, C., and Duyster, J. BCR-ABL gene mutations in relation to clinical resistance of Philadelphia-chromosome-positive leukaemia to STI571: a prospective study. Lancet, 359: 487-491, 2002; each of which is incorporated herein by reference), and further substantiated by crystal structure analysis indicating that some of the mutations observed in resistant patients occur at positions that were predicted to be critical for binding imatinib (Schindler, T., Bornmann, W., Pellicena, P., Miller, W. T., Clarkson, B., and Kuriyan, J. Structural mechanism for STI-571 inhibition of abelson tyrosine kinase. Science, 289: 1938-1942, 2000; Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; each of which is incorporated herein by reference). Specific binding of imatinib to Abl implies a complex interaction to various residues within the ATP-binding site (Schindler, T., Bornmann, W., Pellicena, P., Miller, W. T., Clarkson, B., and Kuriyan, J. Structural mechanism for STI-571 inhibition of abelson tyrosine kinase. Science, 289: 1938-1942, 2000; incorporated herein by reference). In addition to the initially described exchange of threonine at position 315 to isoleucine (Gorre, M. E., Mohammed, M., Ellwood, K., Hsu, N., Paquette, R., Rao, P. N., and Sawyers, C. L. Clinical resistance to STI-571 cancer therapy caused by BCR-ABL gene mutation or amplification. Science, 293: 876-880, 2001; incorporated herein by reference), a number of residues within the BCR-ABL kinase domain were found to be mutated in cases of refractory or relapsed Ph+leukemia (Branford, S., Rudzki, Z., Walsh, S., Grigg, A., Arthur, C., Taylor, K., Herrmann, R., Lynch, K. P., and Hughes, T. P. High frequency of point mutations clustered within the adenosine triphosphate-binding region of BCR/ABL in patients with chronic myeloid leukemia or Ph-positive acute lymphoblastic leukemia who develop imatinib (ST1571) resistance. Blood, 99: 3472-3475, 2002; von Bubnoff, N., Schneller, F., Peschel, C., and Duyster, J. BCR-ABL gene mutations in relation to clinical resistance of Philadelphia-chromosome-positive leukaemia to ST1571: a prospective study. Lancet, 359: 487-491, 2002; Hofmann, W. K., Jones, L. C., Lemp, N. A., de Vos, S., Gschaidmeier, H., Hoelzer, D., Ottmann, O. G., and Koeffler, H. P. Ph (+) acute lymphoblastic leukemia resistant to the tyrosine kinase inhibitor ST1571 has a unique BCR-ABL gene mutation. Blood, 99: 1860-1862, 2002; Roche-Lestienne, C., Soenen-Cornu, V., Grardel-Duflos, N., Lai, J. L., Philippe, N., Facon, T., Fenaux, P., and Preudhomme, C. Several types of mutations of the Abl gene can be found in chronic myeloid leukemia patients resistant to ST1571, and they can pre-exist to the onset of treatment. Blood, 100: 1014-1018, 2002; Shah, N. P., Nicoll, J. M., Nagar, B., Gorre, M. E., Paquette, R. L., Kuriyan, J., and Sawyers, C. L. Multiple BCR-ABL kinase domain mutations confer polyclonal resistance to the tyrosine kinase inhibitor imatinib (ST1571) in chronic phase and blast crisis chronic myeloid leukemia. Cancer Cell, 2: 117-125, 2002; Hochhaus, A., Kreil, S., Corbin, A. S., La Rosee, P., Muller, M. C., Lahaye, T., Hanfstein, B., Schoch, C., Cross, N. C., Berger, U., Gschaidmeier, H., Druker, B. J., and Hehlmann, R. Molecular and chromosomal mechanisms of resistance to imatinib (ST1571) therapy. Leukemia, 16: 2190-2196, 2002; Barthe, C., Cony-Makhoul, P., Melo, J. V., and Mahon, J. R. Roots of clinical resistance to STI-571 cancer therapy. Science, 293: 2163, 2001; Hochhaus, A., Kreil, S., Corbin, A., La Rosee, P., Lahaye, T., Berger, U., Cross, N. C., Linkesch, W., Druker, B. J., Hehlmann, R., Passerini, C., Comeo, G., and D'Incalci, M. Roots of clinical resistance to STI-571 cancer therapy. Science, 293: 2163, 2001; Roumiantsev, S., Shah, N. P., Gorre, M. E., Nicoll, J., Brasher, B. B., Sawyers, C. L., and Van Etten, R. A. Clinical resistance to the kinase inhibitor STI-571 in chronic myeloid leukemia by mutation of Tyr-253 in the Abl kinase domain P-loop. Proc. Natl. Acad. Sci. U.S.A, 99: 10700-10705, 2002; each of which is incorporated herein by reference). The resulting amino acid exchanges can be devided into different categories: First, there are residues that make direct contact to imatinib, where an exchange presumably causes a steric clash with imatinib, such as F311, T315, and F317 (Shah, N. P., Nicoll, J. M., Nagar, B., Gorre, M. E., Paquette, R. L., Kuriyan, J., and Sawyers, C. L. Multiple BCR-ABL kinase domain mutations confer polyclonal resistance to the tyrosine kinase inhibitor imatinib (ST1571) in chronic phase and blast crisis chronic myeloid leukemia. Cancer Cell, 2: 117-125, 2002; Schindler, T., Bornmann, W., Pellicena, P., Miller, W. T., Clarkson, B., and Kuriyan, J. Structural mechanism for STI-571 inhibition of abelson tyrosine kinase. Science, 289: 1938-1942, 2000; Manley, P., Cowan-Jacob, S. W., Fabbro, D., Fendrich, G., Furet, P., Guez, V., Liebetanz, J., Mestan, J., and Meyer, T. Molecular interactions between Glivec and isoforms of the kinase domain of c-Abl kinase. Proc Am Assoc Cancer Res 4196. 2002; each of which is incorporated herein by reference), with T3151 being the most frequent exchange (Shah, N. P., Nicoll, J. M., Nagar, B., Gorre, M. E., Paquette, R. L., Kuriyan, J., and Sawyers, C. L. Multiple BCR-ABL kinase domain mutations confer polyclonal resistance to the tyrosine kinase inhibitor imatinib (ST1571) in chronic phase and blast crisis chronic myeloid leukemia. Cancer Cell, 2: 117-125, 2002; Corbin, A. S., La Rosee, P., Stoffregen, E. P., Druker, B. J., and Deininger, M. W. Several Bcr-Abl kinase domain mutants associated with imatinib mesylate resistance remain sensitive to imatinib. Blood, 2003; each of which is incorporated herein by reference). Second, mutations that affect the nucleotide-binding (P) loop, that has to adopt a specific conformation in order to allow imatinib to bind (Shah, N. P., Nicoll, J. M., Nagar, B., Gorre, M. E., Paquette, R. L., Kuriyan, J., and Sawyers, C. L. Multiple BCR-ABL kinase domain mutations confer polyclonal resistance to the tyrosine kinase inhibitor imatinib (ST1571) in chronic phase and blast crisis chronic myeloid leukemia. Cancer Cell, 2: 117-125, 2002; Veach, D. R., Swendeman, S., Nagar, B., Wisniewski, D., Strife, A., Lambek, C. L., Liu, C., Li, W. W., Bornmann, W. G., Kuriyan, J., Bertino, J. R., and Clarkson, B. Towards picomolar inhibition of Bcr-Abl: Synthesis and evaluation of a focused library of pyrido-[2,3-d]-pyrimidine tyrosine kinase inhibitors guided by X-ray crystallography and molecular modeling. Proc Am Assoc Cancer Res 43, 847. 2002; Manley, P., Cowan-Jacob, S. W., Fabbro, D., Fendrich, G., Furet, P., Guez, V., Liebetanz, J., Mestan, J., and Meyer, T. Molecular interactions between Glivec and isoforms of the kinase domain of c-Abl kinase. Proc Am Assoc Cancer Res 4196. 2002; each of which is incorporated herein by reference). The most frequent exchange falling into this category is E255K/V, followed by Y253H/F (Shah, N. P., Nicoll, J. M., Nagar, B., Gorre, M. E., Paquette, R. L., Kuriyan, J., and Sawyers, C. L. Multiple BCR-ABL kinase domain mutations confer polyclonal resistance to the tyrosine kinase inhibitor imatinib (ST1571) in chronic phase and blast crisis chronic myeloid leukemia. Cancer Cell, 2: 117-125, 2002; Corbin, A. S., La Rosee, P., Stoffregen, E. P., Druker, B. J., and Deininger, M. W. Several Bcr-Abl kinase domain mutants associated with imatinib mesylate resistance remain sensitive to imatinib. Blood, 2003; each of which is incorporated herein by reference). Third, mutations that are located within the activation loop, with H396P/R being the most frequent one (Shah, N. P., Nicoll, J. M., Nagar, B., Gorre, M. E., Paquette, R. L., Kuriyan, J., and Sawyers, C. L. Multiple BCR-ABL kinase domain mutations confer polyclonal resistance to the tyrosine kinase inhibitor imatinib (ST1571) in chronic phase and blast crisis chronic myeloid leukemia. Cancer Cell, 2: 117-125, 2002; Corbin, A. S., La Rosee, P., Stoffregen, E. P., Druker, B. J., and Deininger, M. W. Several Bcr-Abl kinase domain mutants associated with imatinib mesylate resistance remain sensitive to imatinib. Blood, 2003; each of which is incorporated herein by reference). Recently, an in vitro screen of randomly mutagenized variants of BCR-ABL demonstrated that mutations involving critical interdomain contact sites beyond the kinase domain as well can cause resistance to imatinib in vitro (Azam, M., Latek, R. R., and Daley, G. Q. Mechanisms of autoinhibition and STI-571/imatinib resistance revealed by mutagenesis of BCR-ABL. Cell, 112: 831-843, 2003; incorporated herein by reference).

It was recently found that PD166326, PD173955, and PD180970, pyrido-[2,3-d]pyrimidine small molecule inhibitors of ATP-binding, are potent inhibitors of Bcr-Abl (Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; Wisniewski, D., Lambek, C. L., Liu, C., Strife, A., Veach, D. R., Nagar, B., Young, M. A., Schindler, T., Bornmann, W. G., Bertino, J. R., Kuriyan, J., and Clarkson, B. Characterization of potent inhibitors of the Bcr-Abl and the c-kit receptor tyrosine kinases. Cancer Res., 62: 4244-4255, 2002; Dorsey, J. F., Jove, R., Kraker, A. J., and Wu, J. The pyrido[2,3-d]pyrimidine derivative PD180970 inhibits p210Bcr-Abl tyrosine kinase and induces apoptosis of K562 leukemic cells. Cancer Res., 60: 3127-3131, 2000; Veach, D. R., Swendeman, S., Nagar, B., Wisniewski, D., Strife, A., Lambek, C. L., Liu, C., Li, W. W., Bornmann, W. G., Kuriyan, J., Bertino, J. R., and Clarkson, B. Towards picomolar inhibition of Bcr-Abl: Synthesis and evaluation of a focused library of pyrido-[2,3-d]-pyrimidine tyrosine kinase inhibitors guided by X-ray crystallography and molecular modeling. Proc Am Assoc Cancer Res 43, 847. 2002; Veach, D. R., Lambek, C., Liu, C., Beresten, T., Wisniewski, D., Li, W., Wolff, N. C., Ilaria, R. L., Jr., Gelovani, J., Bornmann, W. G., and Clarkson, B. D. Synthesis, in silico, in vitro and in vivo characterization of pyrido[2,3-d]-pyrimidine tyrosine kinase inhibitors. Proc Am Assoc Cancer Res 44. 2003; Strife, A., Wisniewski, D., Liu, C., Lambek, C. L., Darzynkiewicz, Z., Silver, R. T., and Clarkson, B. Direct Evidence That Bcr-Abl Tyrosine Kinase Activity Disrupts Normal Synergistic Interactions Between Kit Ligand and Cytokines in Primary Primitive Progenitor Cells. Mol. Cancer Res., 1: 176-185, 2003; each of which is incorporated herein by reference). PD173955 has been co-crystalized with the kinase domain of murine c-Abl (residues 229-515) (Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; incorporated herein by reference), and PD166326 was crystalized in complex with larger Abl constructs, residues 1-531 of human c-Abl and residues 46-534 of murine c-Abl (Hantschel, O., Nagar, B., Guettler, S., Kretzschmar, J., Dorey, K., Kuriyan, J., and Superti-Furga, G. A myristoyl/phosphotyrosine switch regulates c-Abl. Cell, 112: 845-857, 2003; Nagar, B., Hantschel, O., Young, M. A., Scheffzek, K., Veach, D., Bornmann, W., Clarkson, B., Superti-Furga, G., and Kuriyan, J. Structural basis for the autoinhibition of c-Abl tyrosine kinase. Cell, 112: 859-871, 2003; each of which is incorporated herein by reference). Crystal structure analysis has demonstrated differences in the mode of binding of pyrido-pyrimidines versus imatinib, but at the same time suggested that positions critical for binding of either drug may also overlap (Schindler, T., Bornmann, W., Pellicena, P., Miller, W. T., Clarkson, B., and Kuriyan, J. Structural mechanism for STI-571 inhibition of abelson tyrosine kinase. Science, 289: 1938-1942, 2000; Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; each of which is incorporated herein by reference). Thus, we examined whether pyrido-[2,3-d]pyrimidine derivatives perform superior to imatinib in the potency of inhibition of Bcr-Abl, and whether activity is maintained against clinically relevant mutant forms of BCR-ABL, that cause resistance towards imatinib.

Materials and Methods

Inhibitors

Pyrido-[2,3-d]pyrimidine analogues were synthesized by Darren R. Veach and Wiliam G. Bornmann, Memorial Sloan-Kettering Cancer Center, New York, N.Y., USA. The compounds were dissolved at 10 mM in DMSO and stored at −20° C.

Generation of Cell Lines

Mutations within the kinase domain of Abl in cases of Ph+leukemia resistant to imatinib were identified as described (von Bubnoff, N., Schneller, F., Peschel, C., and Duyster, J. BCR-ABL gene mutations in relation to clinical resistance of Philadelphia-chromosome-positive leukaemia to ST1571: a prospective study. Lancet, 359: 487-491, 2002; incorporated herein by reference). Briefly, RT-PCR fragments derived from clinical samples were cloned into pBlueskript SK+ p185^{bcr-abl} (Stratagene, Amsterdam, NL), and subcloned into pcDNA3.1/Zeo (+) (Invitrogen, Leek, NL) and Mig EGFP (Bai, R. Y., Ouyang, T., Miething, C., Morris, S. W., Peschel, C., and Duyster, J. Nucleophosmin-anaplastic lymphoma kinase associated with anaplastic large-cell lymphoma activates the phosphatidylinositol 3-kinase/Akt antiapoptotic signaling pathway. Blood, 96: 4319-4327, 2000; incorporated herein by reference). Presence of single point mutations was verified by sequencing. Ba/F3 cells were maintained in RPMI 1640 growth media (Gibco, Karlsruhe, Germany) containing 10% fetal calf serum (Gibco) and P/S (Gibco) (200 U penicillin per mL and 200 μg streptomycin per mL). Parental cells were cultured in presence of 2 ng/mL interleukin-3 (IL-3, R&D, Wiesbaden, Germany). IL-3 independent Ba/F3 cells expressing wild-type and mutant BCR-ABL were generated by electroporation and transformed upon withdrawal of IL-3. Expression of Bcr-Abl was confirmed by western blot and flow-cytometric analysis.

Proliferation

Proliferation was measured using a MTS tetrazolium-based method by absorption of formazam at 490 nm (CellTiter 96, Promega, Madison, Wis., USA). Measures were taken as triplicates after 24 and 48 hours of culture without and in the presence of inhibitor at the indicated concentrations.

Tyrosine Phosphorylation

Ba/F3 cells were cultured for 2,5 hours without and in presence of inhibitor at the indicated concentrations. Cell lysis, SDS-PAGE and immunoblotting were done as described previously.⁴² Anti-Abl antibodies were obtained from Pharmingen (8E9) (BD Biosciences, Heidelberg, Germany) and Calbiochem-Novabiochem (Ab3) (Schwalbach, Germany). Antibodies to phosphotyrosine were purchased from Upstate Biotechnology (4G10) (Biozol, Eching, Germany) and Transduction (PY20) (BD Biosciences, Heidelberg, Germany). Bands were visualized using the ECL system (Amersham, Braunschweig, Germany).

In Vitro Kinase Assays

Kinase assays were carried out using the purified catalytic domain of Abl (Schindler, T., Bornmann, W., Pellicena, P., Miller, W. T., Clarkson, B., and Kuriyan, J. Structural mechanism for STI-571 inhibition of abelson tyrosine kinase. Science, 289: 1938-1942, 2000; incorporated herein by reference). A continuous spectrophotometric assay was used to measure kinase activity, as described previously (Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; incorporated herein by reference). Reactions contained 100 mM Tris-HCl (pH 7.5), 10 mM MgCl2, 0.5 mM ATP, 1 mM phosphoenolpyruvate, 0.28 mM NADH, 89 units/ml pyruvate kinase, 124 units/ml lactate dehydrogenase, 2% DMSO, 0.5 mM peptide substrate (AEEEIYGEFEAKKKKG), and varying concentrations of inhibitors. The reactions were carried out in duplicate at 30° C. and were initiated by the addition of 10 nM Abl catalytic domain.

Detection of Apoptosis

Ba/F3cells were cultured at a density of 1×10⁵/mL without and in presence of inhibitor at the indicated concentrations. At the indicated time points, 5×10⁴ cells were washed twice in phosphate-buffered saline, resuspended in 100 μL annexin V binding buffer (Pharmingen, BD Biosciences, Heidelberg, Germany), and stained with 2 μL annexin V-PE (Pharmingen, BD Biosciences, Heidelberg, Germany) for 20 minutes at RT. Thereafter, 5 μL of a 100 μg/mL 7-amino actinomycin D stock solution (Sigma-Aldrich Chemie GmbH, Steinheim, Germany) were added. Annexin V-positive cells were measured by flourescence-activated cell sorting analysis (FACS) using a Coulter EPICS XL-MCL four-colour cytometer (Beckman Coulter GmbH, Krefeld, Germany).

Results

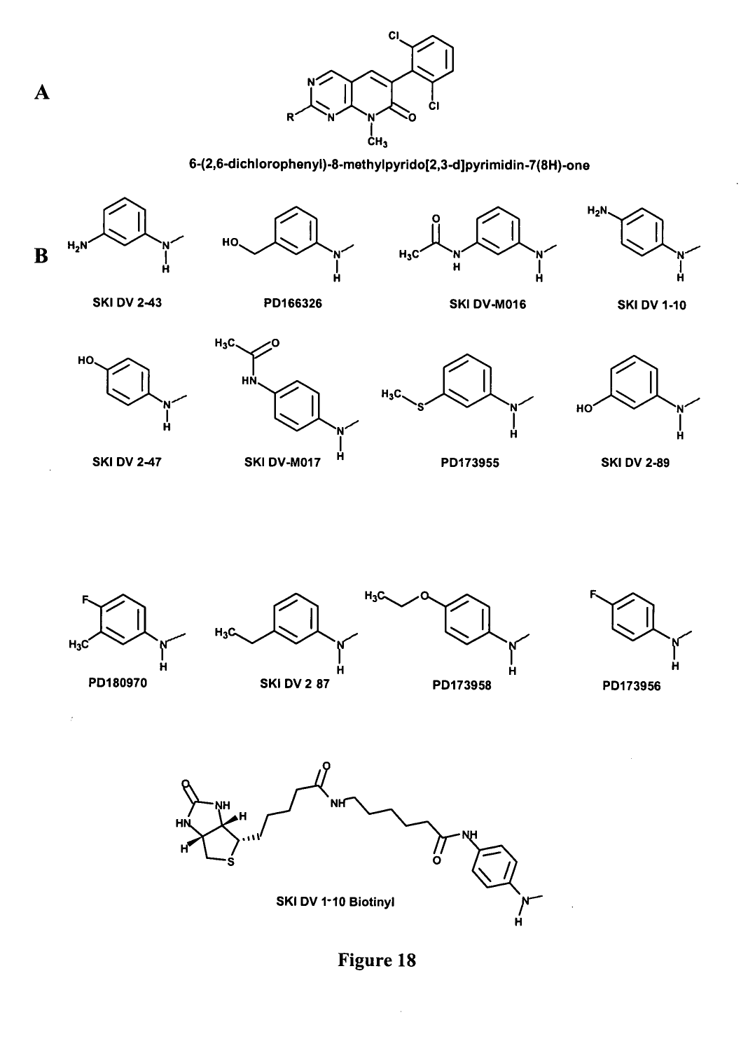

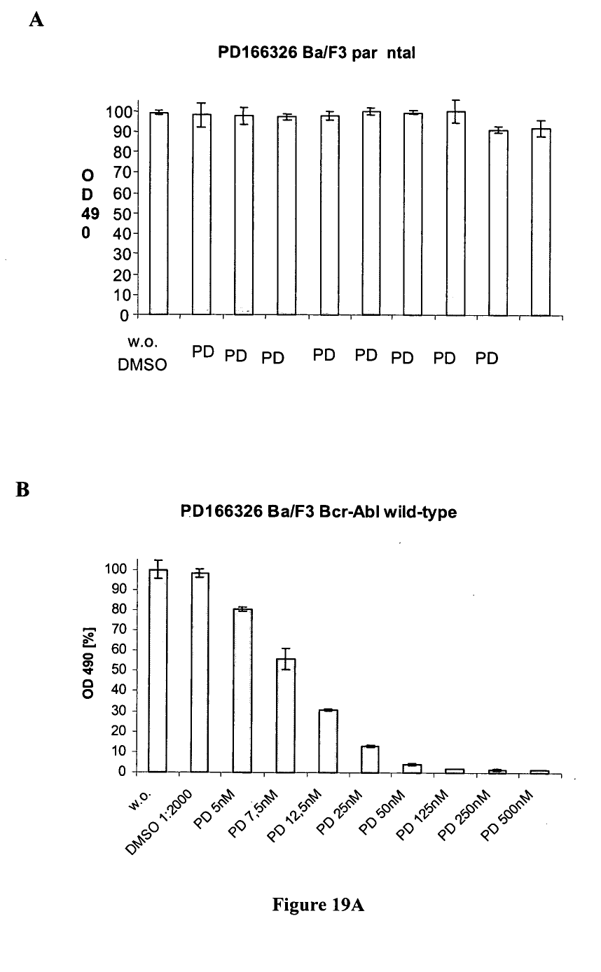

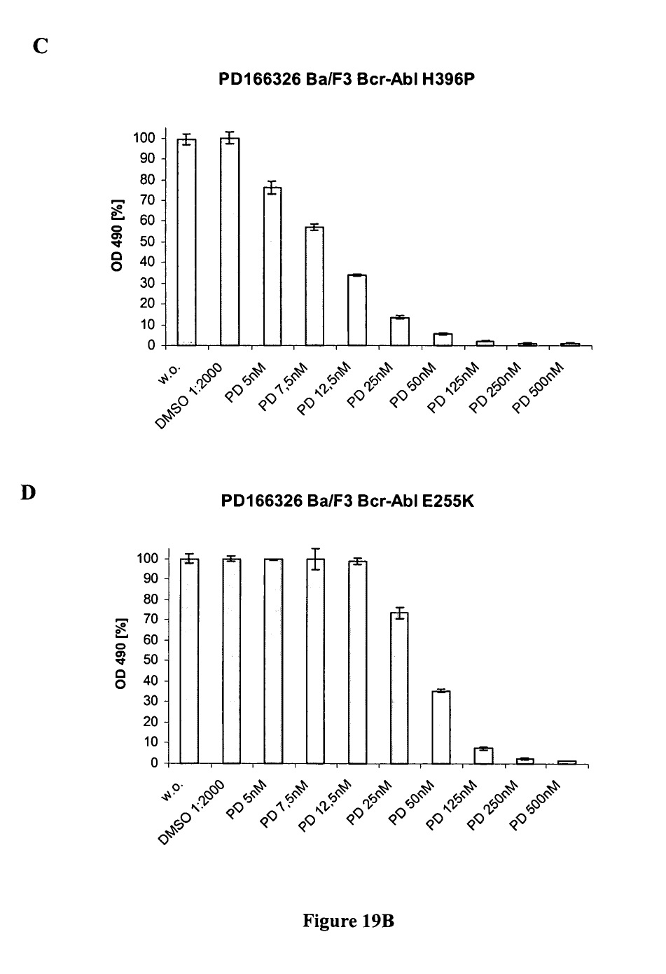

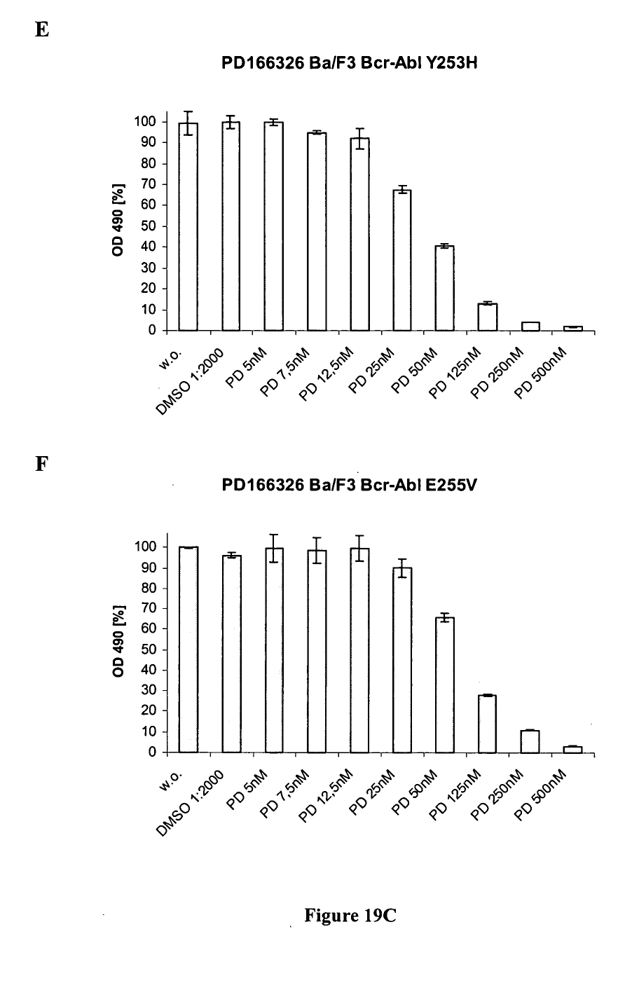

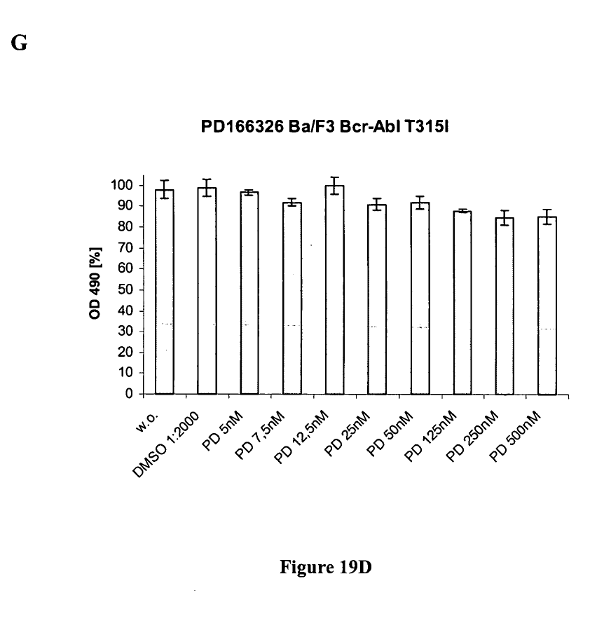

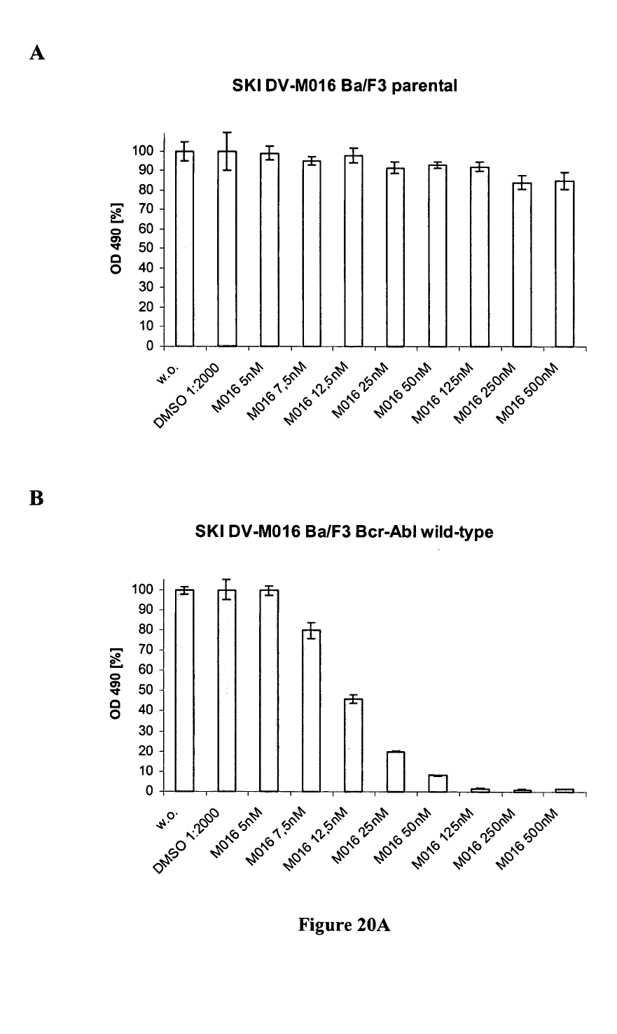

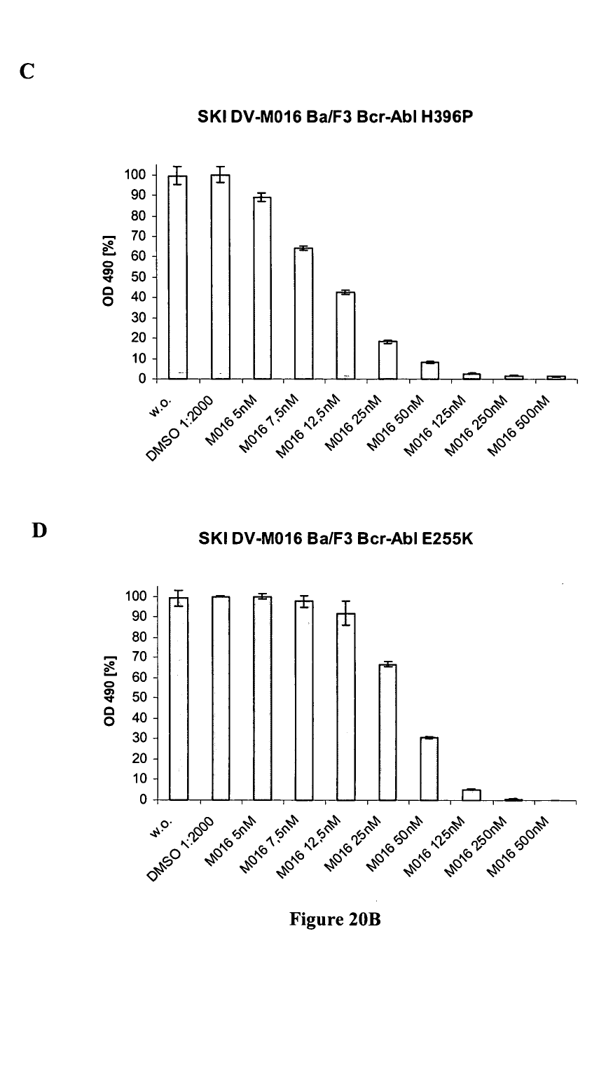

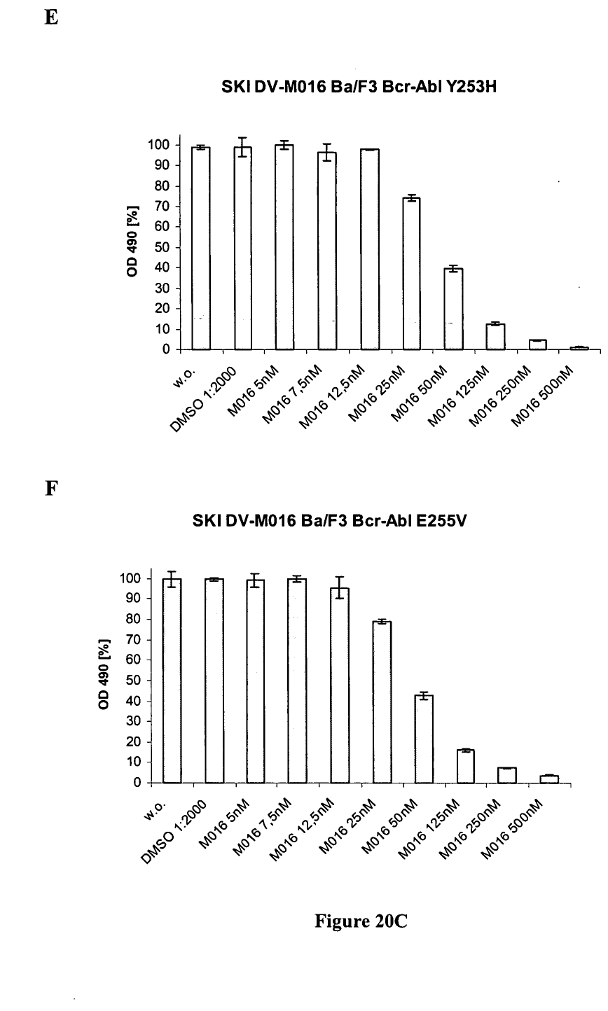

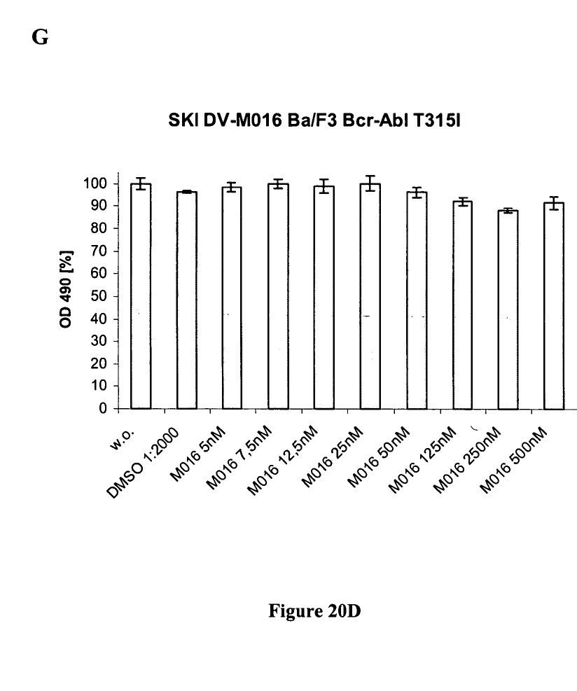

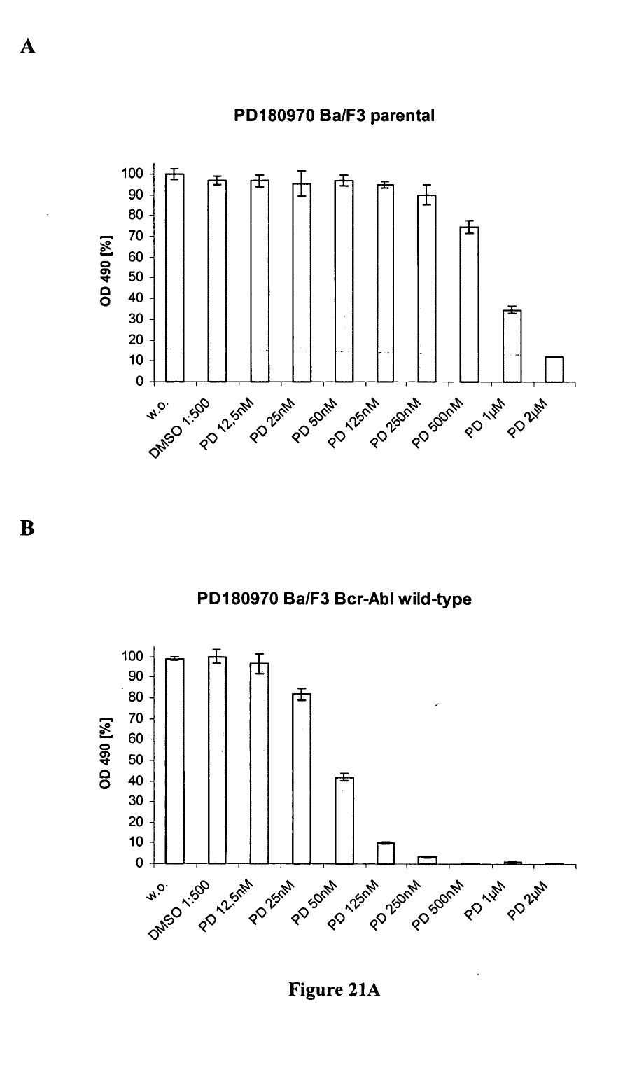

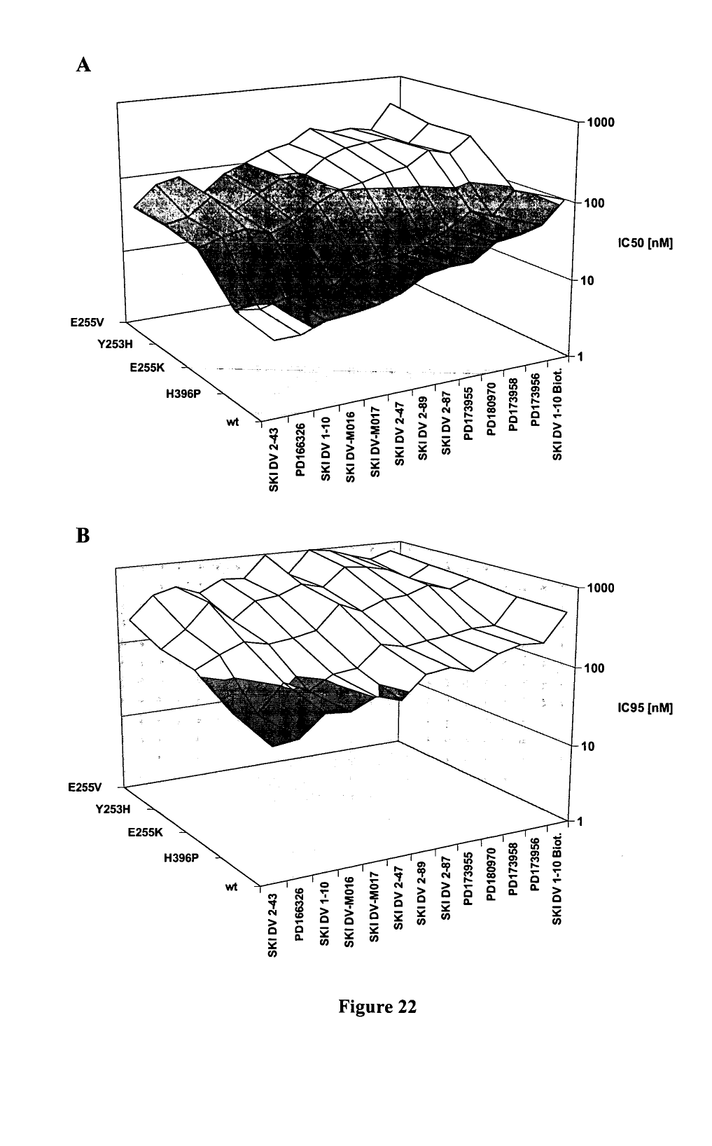

Pyrido-pyrimidines specifically inhibit the growth of Bcr-Abl transformed cells. Thirteen pyrido-[2,3-d]pyrimidine analogues were examined that differed in the substituents located at position 2 of the pyrimidine-ring (see FIG. 18). Tetrazolium-based proliferation assays with Ba/F3 cells transformed with Bcr-Abl wild-type were performed. PD166326 and SKI DV-M016 were the most inhibitory derivatives tested and exhibited IC₅₀-values of 9 nM and 12 nM, and IC₉₅ values of 45 nM and 75 nM, respectively after 48 hours of culture (see FIGS. 19B and 20B). IC₅₀ values for growth inhibition covered a range from 9 nM (SKI DV-2-43 and PD166326) to 115 nM (SKI DV 1-10 biotinyl) with a mean IC₅₀ of 32.5 nM. Most of the compounds displayed IC₅₀ values in the range of 10 nM to 100 nM in wild-type Bcr-Abl expressing cells after 48 hours of culture (see FIG. 22A, front row, purple area). Ninety-five percent of growth inhibition in Bcr-Abl wild-type expressing Ba/F3 cells required a 3.8 to 7.7 fold (mean 5.45 fold) increase in concentrations of inhibitors compared to IC₅₀ values, resulting in IC₉₅ values for growth inhibition that were between 40 nM (SKI DV 2-43) and 515 nM (SKI DV 1-10 biotinyl, see FIG. 22B, front row). Largely similar results were obtained in 32D cells expressing Bcr-Abl wild-type. Specific inhibition of cells expressing wild-type Bcr-Abl occured at a wide range of concentrations. The most active compounds exhibited unspecific inhibition of parental cells with IC₅₀ values that were 100 fold higher than in cells transformed with wild-type Bcr-Abl.

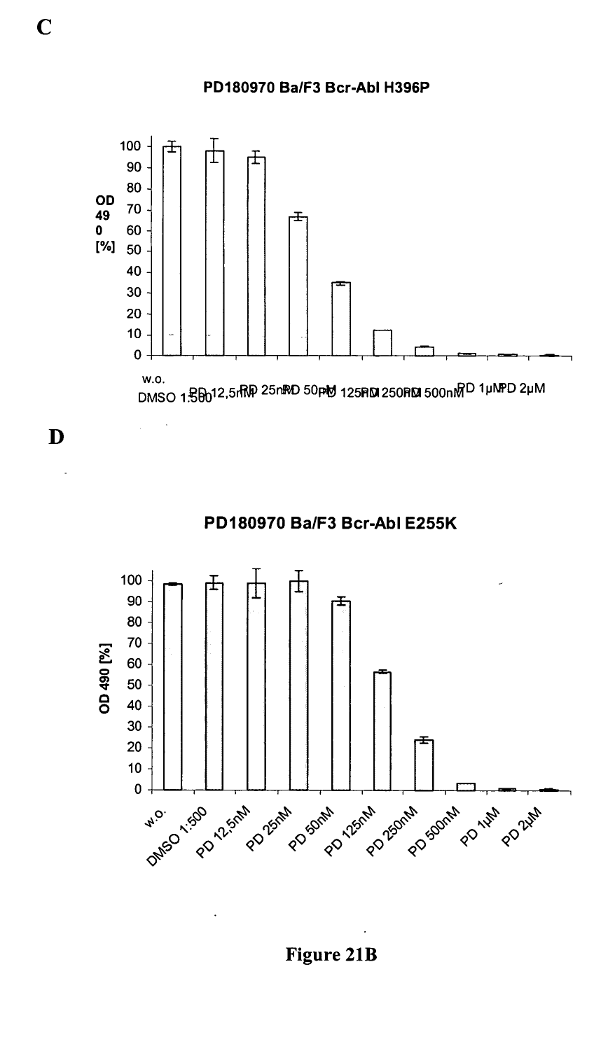

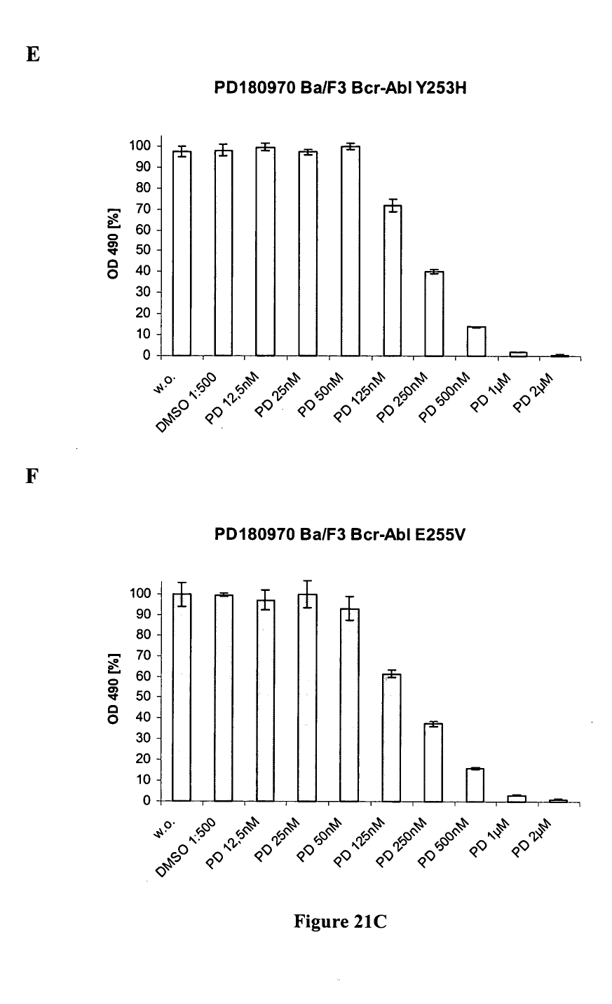

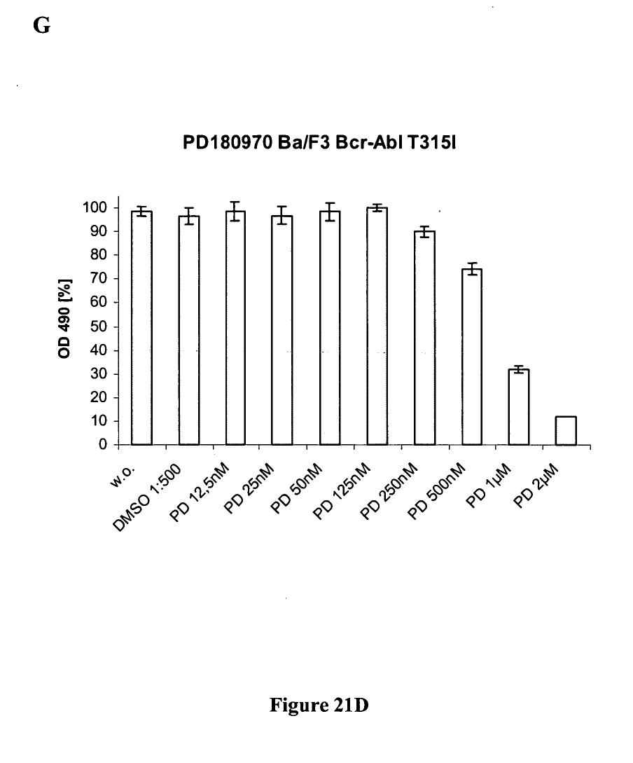

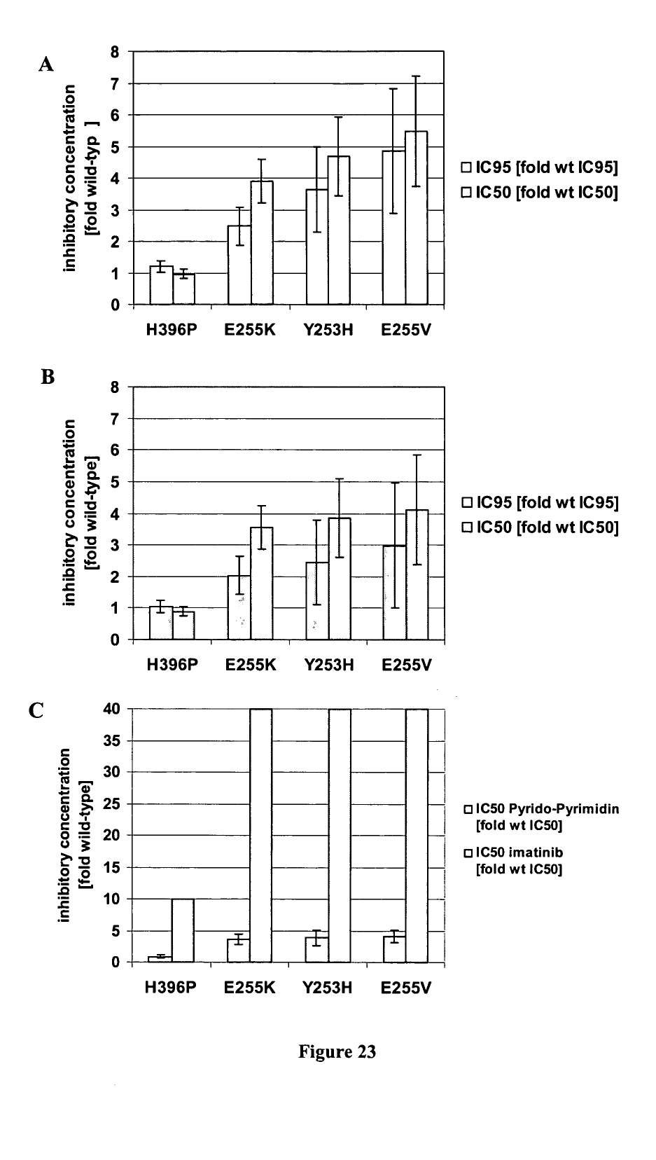

Differential growth inhibition of wild-type versus mutant Bcr-Abl. When Bcr-Abl wild-type and the activation loop mutant H396P were compared, both IC₅₀, and IC₉₅ values were identical for all pyrido-pyrimidines tested (compare FIGS. 19B and 19C for PD166326, FIGS. 20B and 20C for SKI DV-M016, first and second rows in FIGS. 22A and 22B, and FIGS. 23A and 23B, first two columns). In contrast, a 50 percent growth inhibition of the activation loop mutant H396P required a 10-fold increase in imatinib concentrations (FIG. 23C, second column). Specific inhibition of a Bcr-Abl dependent phenotype was also observed when nucleotide-binding loop (P loop) mutations were introduced in Ba/F3 cells. All compounds exhibited an efficient and specific suppression of growth in cells expressing the P loop mutants E255K (FIGS. 19D, 20D, 22A and B, third row), Y253H (FIGS. 19E, 20E, 22A and B, fourth row), and E255V (FIGS. 19F, 20F, 22A and B, fifth row), whereas IC₅₀ values for inhibition of P loop mutants with imatinib were not reached even with a 40-fold increase in imatinib concentrations (FIG. 23C, columns 4, 6, and 8) (von Bubnoff, N., Schneller, F., Peschel, C., and Duyster, J. BCR-ABL gene mutations in relation to clinical resistance of Philadelphia-chromosome-positive leukaemia to ST1571: a prospective study. Lancet, 359: 487-491, 2002; incorporated herein by reference). In opposition to activation loop and P loop mutants, none of the substances was capable of suppressing growth in Ba/F3 cells expressing T3151 (see FIGS. 19G and 20G). When the IC₅₀ and IC₉₅ values for growth inhibition in wild-type and mutant BCR-ABL expressing cells were compared, the fold increase in inhibitor concentration necessary to compensate the lower sensitivity of P loop mutants was 3.9/2.5 fold for IC50/95 in E255K, 4.7/3.6 fold for Y253H, and 5.5/4.9 fold for E255V (see FIG. 23A). When a subgroup of five inhibitors was analysed (SKI DV-M016, SKI DV 2-87, PD173958, PD173956, and SKI DV 1-10-biotinyl), the mean fold increase in substance concentration corresponding to IC₉₅ values of the least sensitive mutant E255V compared to wild-type decreased to three (range 1.5 to 4.7, see FIG. 23B). Comparing this subgroup with the remaining inhibitors, the smaller increment in IC₉₅ values from wild-type to P loop mutants is represented by a lower slope along the Z-axis, and represented by insections along the X-axis in FIG. 22B. In comparison to the most active pyrido-pyrimidines, PD180970 required higher concentrations for inhibition of growth, and displayed unspecific inhibition of parental Ba/F3 cells at concentrations, that were necessary for effective growth suppression of P loop mutants (see FIG. 21).

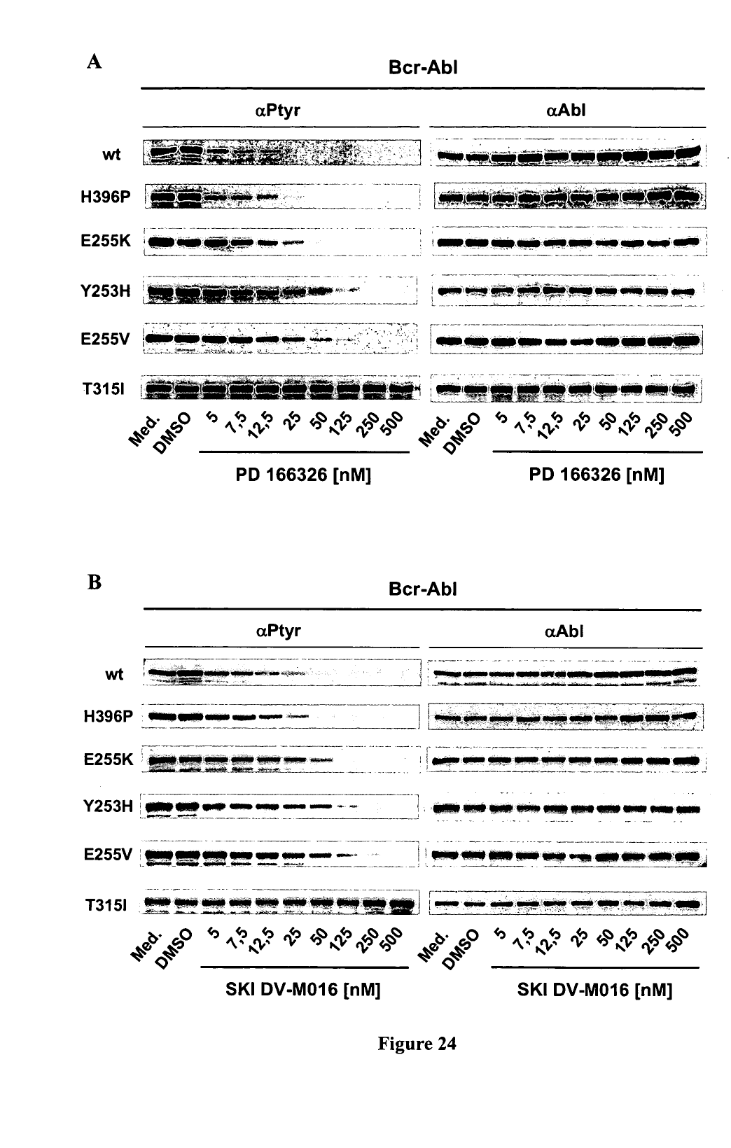

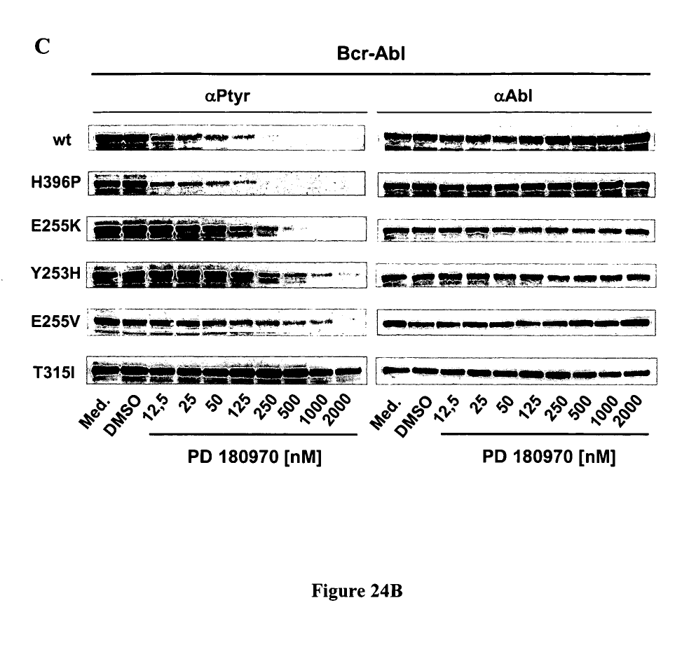

Growth suppression is accompanied by inhibition of Bcr-Abl kinase activity. To investigate whether the observed effects on cell growth resulted from inhibition of Bcr-Abl kinase activity, we cultered parental Ba/F3 cells and Ba/F3 cells expressing either wild-type or mutant forms of Bcr-Abl in the presence of PD166326, SKI DV-MO 16 and PD180970 at different concentrations, and analysed cellular extracts for the phosphotyrosine content of Bcr-Abl. Inhibition of cellular growth correlated well with the disappearance of Bcr-Abl tyrosine phosphorylation (see FIG. 24). A strong effect was observed using PD166326 in Ba/F3 cells expressing Bcr-Abl wild-type, where the IC₅₀ value for phosphorylation was below 5 nM (see FIG. 24A, top panel), paralleling the observed effects on growth (compare FIG. 20B). In cells expressing the activation loop mutant H396P, the effects of PD166326 again were identical to wild-type (see FIG. 24A, second panel). As for Bcr-Abl wild-type and H396P, tyrosine phosphorylation of the P loop mutants E255K, Y253H, and E255V disappeared at concentrations, that were in between IC₅₀ and IC₉₅ values for growth inhibition: At 125 nM in E255K, and at 250 nM in Y253H and E255V, respectively (see FIG. 24A). As expected from the growth assays, activity of Bcr-Abl/T3151 was not affected (see FIG. 24A, bottom panel). SKI DV-MO 16 performed similar, but inhibition of Bcr-Abl tyrosine phosphorylation required slightly higher concentrations compared to PD166326 (see FIG. 24B). This was again in line with the effects of SKI DV-MO 16 on cell growth (compare FIG. 20). Compatible with its lower activity in cellular growth suppression (see FIG. 21), the application of PD180970 necessitated higher concentrations for Bcr-Abl inhibition. Thus, complete disappearance of phoysphotyrosine Bcr-Abl was seen at 250 nM for wild-type and H396P, 1 μM for E255K, and 2 μM for Y253H and E255V, respectively. As observed for PD166326 and SKI DV-M016, PD180970 did not impair activity of Bcr-Abl/T3151 (see FIG. 23C).

Inhibition of wild-type Bcr-Abl was further validated by in vitro kinase assays using purified Abl catalytic domain. The resulting IC₅₀ values for SKI DV-M016, PD166326, and SKI DV 2-43 (see Table 1) were in accordance with the observed cellular effects.

TABLE 1
IC₅₀ values of Abl kinase assays for the most active pyrido-pyrimidines.
In vitro kinase assays were performed using 10 nM purified Abl catalytic
domain. Initial rates were measured, and IC₅₀ values were determined by
non-linear regression analysis.

| | IC50 [nM] |
|---|---|
| | (mean ± SEM) |
| SKI DV-M016 | 4.2 ± 0.6 |
| PD166326 | 4.3 ± 0.8 |
| SKI DV 2-43 | 4.3 ± 0.5 |

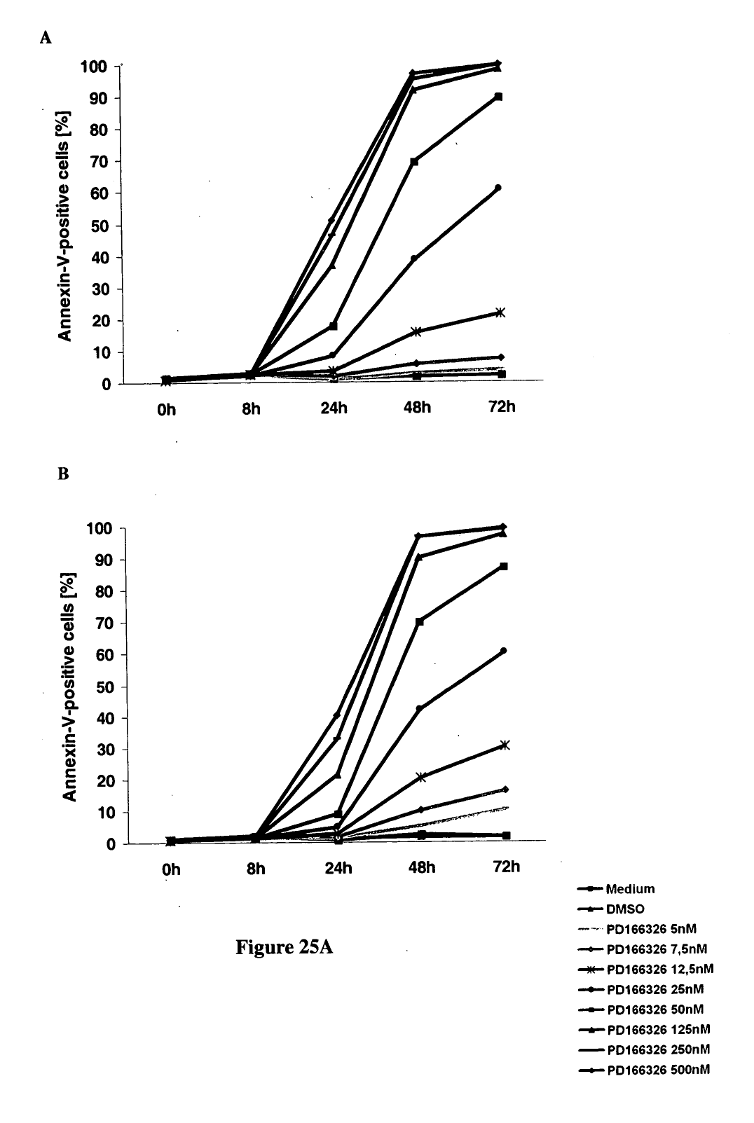

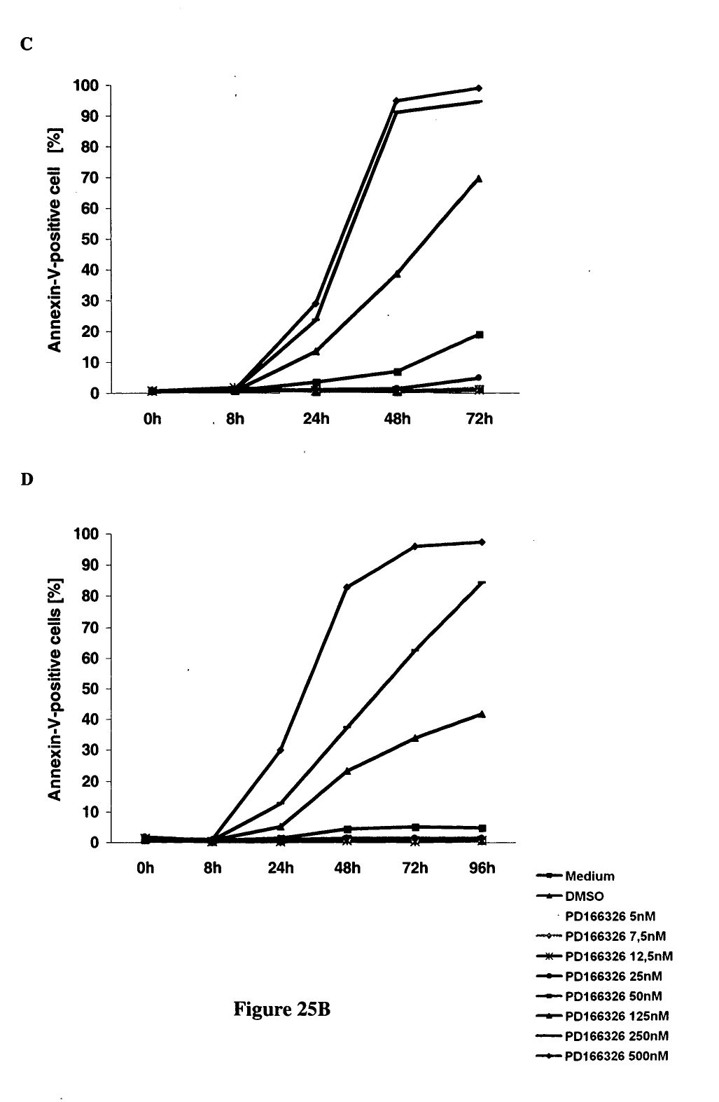

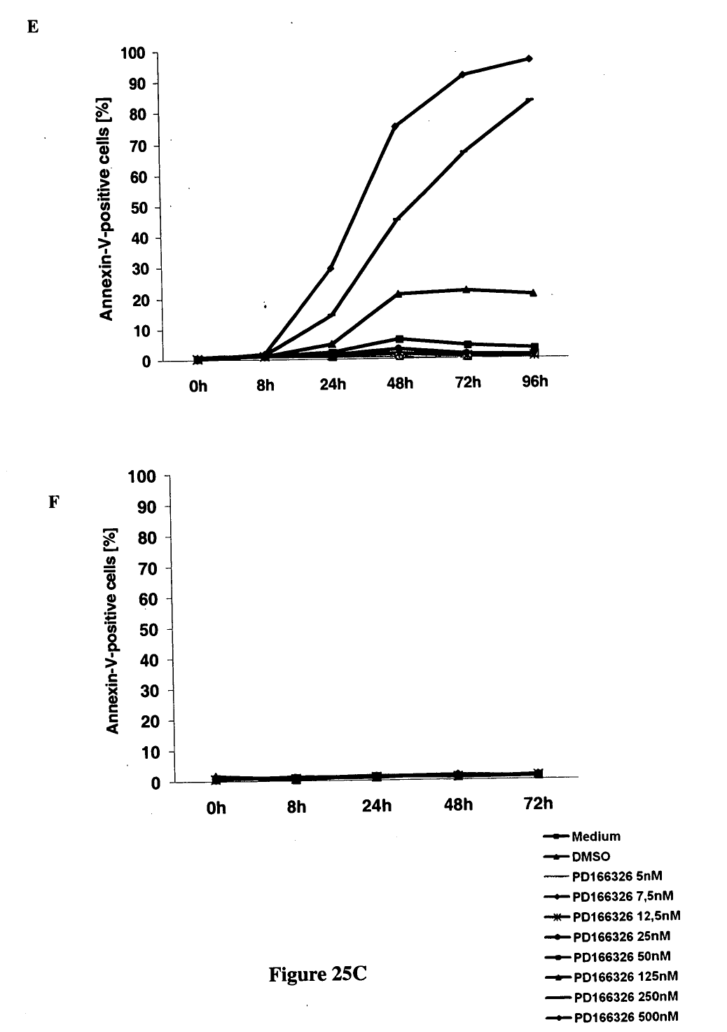

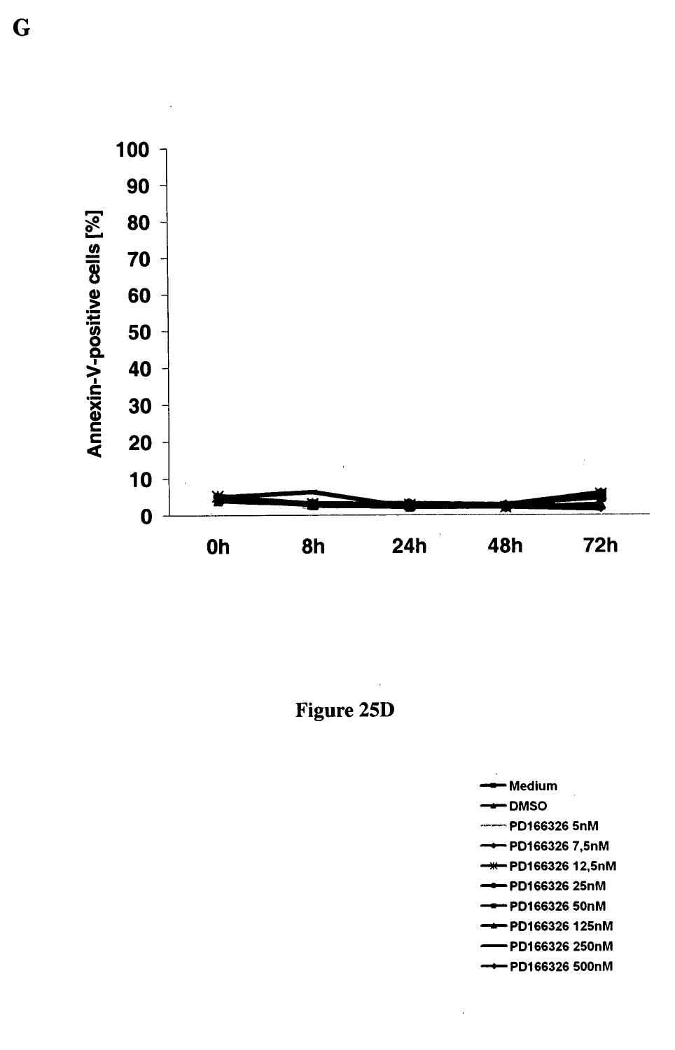

PD166326 specifically induces apoptosis in Ba/F3 cells expressing wild-type and mutant Bcr-Abl. We next examined whether inhibition of Bcr-Abl by a pyrido-pyrimidine gives rise to apoptotic cell death in a Bcr-Abl-dependent phenotype expressing wild-type and mutant forms of Bcr-Abl. PD166326 initiated apoptosis in Ba/F3 cells expressing wild-type (see FIG. 25A) as well as activation loop (FIG. 25B) and P loop (FIGS. 25C, D, and E) mutant forms of Bcr-Abl, as determined by detection of Annexin V-positive cells. Viability of parental Ba/F cells (see FIG. 25G) and cells expressing Bcr-Abl/T3151 (FIG. 25F) was not affected. The content of Annexin V-positive cells was in accordance with the fraction of dead cells as measured by trypan exclusion. Relative sensitivity corresponded to the activity of PD166326 seen in growth and phosphorylation assays, and concentrations that blocked autophosphorylation of Bcr-Abl and inhibited cellular proliferation, at the same time instituted apoptosis.

Discussion

Like imatinib, pyrido-pyrimidines bind to the ATP-binding site, a highly conserved nucleotide-binding pocket within the kinase domain of protein tyrosine kinases, thereby blocking access of ATP (Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; each of which is incorporated herein by reference). Crystal structure analysis of Abl in complex with PD173955 and imatinib suggested that the mode of binding to Abl may differ between 2-phenylaminopyrimidine-type (imatinib) and pyrido-[2,3-d]pyrimidine-type compounds (Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; incorporated herein by reference). The activation loop (residues 381-402 in Abl), a highly conserved region in most kinases located at the amino-terminal of the ATP-binding site, controls catalytic activity by switching between different states in a phosphorylation-dependent manner. Imatinib exclusively binds the closed conformation (Schindler, T., Bornmann, W., Pellicena, P., Miller, W. T., Clarkson, B., and Kuriyan, J. Structural mechanism for STI-571 inhibition of abelson tyrosine kinase. Science, 289: 1938-1942, 2000; Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; incorporated herein by reference). In this conformation, the activation loop folds Y393, the major autophosphorylation site of Abl, towards the active site, superseding bound substrate, and the kinase is inactive. If Y393 gets phosphorylated, the activation loop adopts the open conformation, and the active site is accessible for substrate phosphorylation (Schindler, T., Bornmann, W., Pellicena, P., Miller, W. T., Clarkson, B., and Kuriyan, J. Structural mechanism for STI-571 inhibition of abelson tyrosine kinase. Science, 289: 1938-1942, 2000; Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; incorporated herein by reference). In contrast, the pyrido-pyrimidine PD173955, one of the compounds tested here, was co-crystalized with Abl with the activation loop in an open conformation, and inhibited Abl independent of the phosphorylation state of Abl, suggesting that PD173955 binds irrespective of the conformation of the activation loop (Schindler, T., Bornmann, W., Pellicena, P., Miller, W. T., Clarkson, B., and Kuriyan, J. Structural mechanism for STI-571 inhibition of abelson tyrosine kinase. Science, 289: 1938-1942, 2000; Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; each of which is incorporated herein by reference). This observation can explain several findings: A much more potent inhibition of wild-type Abl than with imatinib is obviously a common feature of pyrido-pyrimidines. The most active pyrido-pyrimidines were by a factor of 28 more active in suppressing the growth of, and by a factor of 50 more active in blocking Bcr-Abl autophosphorylation in cells expressing wild-type Bcr-Abl than imatinib (IC₅₀ proliferation: PD166326 and SKI DV 2-43: 9 nM, imatinib: 250 nM; IC₅₀ Bcr-Abl autophosphorylation: PD166326 and SKI DV 2-43: 4 nM, imatinib: 200 nM). Imatinib, although making more interactions with Abl, may lose some of its binding energy for stabilizing the activation loop in the closed conformation (Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; incorporated herein by reference). The activation loop mutant H396P, while causing an increase in IC₅₀ for imatinib by a factor of 10 (see FIG. 23C), is inhibited as effectively as wild-type Bcr-Abl by all pyrido-pyrimidines tested here (FIG. 23A). As we have proposed earlier, an exchange of the histidine at position 396 may lead to an extended conformation of the activation loop that impairs binding of imatinib (von Bubnoff, N., Schneller, F., Peschel, C., and Duyster, J. BCR-ABL gene mutations in relation to clinical resistance of Philadelphia-chromosome-positive leukaemia to ST1571: a prospective study. Lancet, 359: 487-491, 2002; von Bubnoff, N., Peschel, C., and Duyster, J. Resistance of Philadelphia-chromosome positive leukemia towards the kinase inhibitor imatinib (ST1571, Glivec): a targeted oncoprotein strikes back. Leukemia, in press: 2003; each of which is incorporated herein by reference). A pyrido-pyrimidine-type inhibitor does not require a specific conformation of the activation loop to bind. Therefore, inhibition of H396P by pyrido-pyrimidines is possible in the presence of H396P. In a previous study, cellular IC₅₀ values for PD173955, PD166326, and PD180970 in cells expressing wild-type Bcr-Abl demonstrated to be lower than the IC₅₀ values reported here (Wisniewski, D., Lambek, C. L., Liu, C., Strife, A., Veach, D. R., Nagar, B., Young, M. A., Schindler, T., Bornmann, W. G., Bertino, J. R., Kuriyan, J., and Clarkson, B. Characterization of potent inhibitors of the Bcr-Abl and the c-kit receptor tyrosine kinases. Cancer Res., 62: 4244-4255, 2002; incorporated herein by reference). In that report, the IC₅₀ value for [³H]-thymidine incorporation of glycophorin A negative (R10 negative) MO7e/p210 cells with PD166326 was 0,4 nM, with PD1739551 nM, and with imatinib 35-40 nM; in our study, the respective IC₅₀ values were 9 nM, 30 nM, and 200 nM, respectively. Several reasons may explain this finding. First, different assays were employed. We used tetrazolium-based proliferation assays. A direct comparison of terazolium- and [³H] thymidine-based proloferation assays with R10(−) cells and PD166326 revealed that incorporation of [³H] thymidine was three to four fold more sensitive in comparison to tetrazolium-based assays (data not shown). Second, compared to other Bcr-Abl-positive cell lines, sublines of MO7e/p210 cells seem to be be particularly sensitive to inhibition of Bcr-Abl (Wisniewski, D., Lambek, C. L., Liu, C., Strife, A., Veach, D. R., Nagar, B., Young, M. A., Schindler, T., Bornmann, W. G., Bertino, J. R., Kuriyan, J., and Clarkson, B. Characterization of potent inhibitors of the Bcr-Abl and the c-kit receptor tyrosine kinases. Cancer Res., 62: 4244-4255, 2002; each of which is incorporated herein by reference).

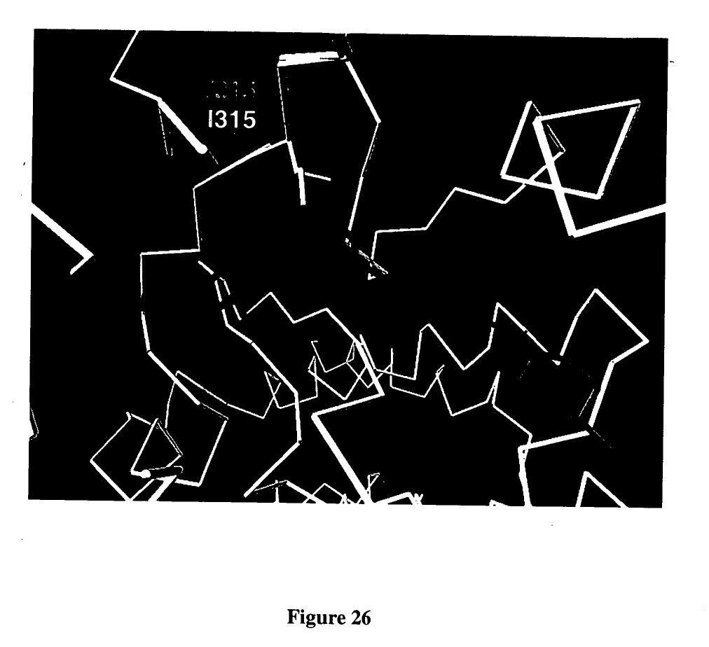

In marked contrast to Bcr-Abl wild type, pyrido-pyrimidines had no advantage over imatinib for inhibition of Bcr-Abl/T3151. None of the compounds inhibited proliferation of Ba/F3 or 32D cells transformed by Bcr-Abl/T3151 or inhibited autophosphorylation of Bcr-Abl/T3151 in Ba/F3 cells. Both imatinib, and the pyrido-pyrimidine PD173955 interact with the threonine at position 315 of c-Abl. Imatinib forms a H-bond with T315 not present with 1315 (Schindler, T., Bornmann, W., Pellicena, P., Miller, W. T., Clarkson, B., and Kuriyan, J. Structural mechanism for STI-571 inhibition of abelson tyrosine kinase. Science, 289: 1938-1942, 2000; Nagar, B., Bornmann, W. G., Pellicena, P., Schindler, T., Veach, D. R., Miller, W. T., Clarkson, B., and Kuriyan, J. Crystal structures of the kinase domain of c-Abl in complex with the small molecule inhibitors PD173955 and imatinib (STI-571). Cancer Res., 62: 4236-4243, 2002; each of which is incorporated herein by reference), and an exchange to isoleucine at this position adds an extra hydrocarbon group in its side chain presumably causing a steric clash with both type of compounds (see FIG. 26). Bcr-Abl/T3151 may therefore be insensitive not only to the compounds tested here, but as well to other pyrido-pyrimidines carrying a dichlorphenyl substituent at the same position. If a modification of the dichlorphenyl substituent would be sufficient to circumvent the clash with isoleucine side chain of T3151 remains to be shown. Alternatively, presence of T3151 may necessitate inhibition of biologically relevant proteins other than Bcr-Abl, e.g., inhibition of the molecular chaperone HSP90, that was reported to induce degradation of Bcr-Abl/T3151 (Gorre, M. E., Ellwood-Yen, K., Chiosis, G., Rosen, N., and Sawyers, C. L. BCR-ABL point mutants isolated from patients with imatinib mesylate-resistant chronic myeloid leukemia remain sensitive to inhibitors of the BCR-ABL chaperone heat shock protein 90. Blood, 100: 3041-3044, 2002; incorporated herein by reference). Parallel to our investigations, another group reported that one of the most active compounds tested here, PD166326, was active in Bcr-Abl E255K, while Bcr-Abl T315I was largely resistant. This is in line with our results for PD166326 in those two particular mutants. However, that report described some activity of PD166326 in growth assays of Ba/F3 Bcr-Abl T3151, with an IC₅₀ value of 150 nM. In contrast, as stated above, we were not able to see any difference between Bcr-Abl T3151 and their parental counterparts looking at proliferation, Bcr-Abl autophosphorylation, and apoptosis in different cell lines.

The region that is most frequently mutated in cases of imatinib resistant Ph+leukemia is the nucleotide-binding (P) loop, a highly conserved region of the kinase domain involved in ATP binding, with Y253 and E255 being affected in the majority of cases (Shah, N. P., Nicoll, J. M., Nagar, B., Gorre, M. E., Paquette, R. L., Kuriyan, J., and Sawyers, C. L. Multiple BCR-ABL kinase domain mutations confer polyclonal resistance to the tyrosine kinase inhibitor imatinib (ST1571) in chronic phase and blast crisis chronic myeloid leukemia. Cancer Cell, 2: 117-125, 2002; Corbin, A. S., La Rosee, P., Stoffregen, E. P., Druker, B. J., and Deininger, M. W. Several Bcr-Abl kinase domain mutants associated with imatinib mesylate resistance remain sensitive to imatinib. Blood, 2003; Branford, S., Rudzki, Z., Walsh, S., Parkinson, I., Grigg, A., Szer, J., Taylor, K., Herrmann, R., Seymour, J. F., Arthur, C., Joske, D., Lynch, K., and Hughes, T. The detection of BCR-ABL mutations in imatinib-treated CML patients is virtually always accompanied by clinical resistance and mutations in the ATP phosphate-binding loop (P-loop) are associated with a poor prognosis. Blood, 2003; each of which is incorporated herein by reference). The P loop mutations examined here are highly resistant to imatinib with cellular IC50 values greater than 40-fold higher than wild-type Bcr-Abl (see FIG. 6C) (von Bubnoff, N., Schneller, F., Peschel, C., and Duyster, J. BCR-ABL gene mutations in relation to clinical resistance of Philadelphia-chromosome-positive leukaemia to ST1571: a prospective study. Lancet, 359: 487-491, 2002; each of which is incorporated herein by reference), and by far exceed concentrations measured in vivo (Druker, B. J. and Mauro, M. J. ST1571: targeting BCR-ABL as therapy for CML. Oncologist., 6: 233-238, 2001; incorporated herein by reference). P loop mutations in patients with resistance towards imatinib were associated with a particular poor prognosis (Branford, S., Rudzki, Z., Walsh, S., Parkinson, I., Grigg, A., Szer, J., Taylor, K., Herrmann, R., Seymour, J. F., Arthur, C., Joske, D., Lynch, K., and Hughes, T. The detection of BCR-ABL mutations in imatinib-treated CML patients is virtually always accompanied by clinical resistance and mutations in the ATP phosphate-binding loop (P-loop) are associated with a poor prognosis. Blood, 2003; incorporated herein by reference). Moreover, Y272F in c-Abl, corresponding to Y253F in Bcr-Abl, activates transformation by c-Abl (Allen, P. B. and Wiedemann, L. M. An activating mutation in the ATP binding site of the ABL kinase domain. J. Biol. Chem., 271: 19585-19591, 1996; incorporated herein by reference), and these mutations give rise to an increase of catalytic activity of c-Abl and Bcr-Abl in vivo (Roumiantsev, S., Shah, N. P., Gorre, M. E., Nicoll, J., Brasher, B. B., Sawyers, C. L., and Van Etten, R. A. Clinical resistance to the kinase inhibitor STI-571 in chronic myeloid leukemia by mutation of Tyr-253 in the Abl kinase domain P-loop. Proc. Natl. Acad. Sci. U.S.A, 99: 10700-10705, 2002; Allen, P. B. and Wiedemann, L. M. An activating mutation in the ATP binding site of the ABL kinase domain. J. Biol. Chem., 271: 19585-19591, 1996; Corbin, A. S., Buchdunger, E., Pascal, F., and Druker, B. J. Analysis of the structural basis of specificity of inhibition of the Abl kinase by ST1571. J. Biol. Chem., 277: 32214-32219, 2002; each of which is incorporated herein by reference). In contrast, T3151 has been demonstrated to decrease Bcr-Abl kinase activity to 60% of wild-type (Corbin, A. S., Buchdunger, E., Pascal, F., and Druker, B. J. Analysis of the structural basis of specificity of inhibition of the Abl kinase by ST1571. J. Biol. Chem., 277: 32214-32219, 2002; each of which is incorporated herein by reference).

In contrast to imatinib, all pyrido-pyrimidines tested here were potent inhibitors of P loop mutants. Cellular IC₉₅ values for inhibition with the most active compound SKI DV 2-43 were 110 nM for inhibition of E255K, 125 nM for Y253H, and 200 nM for inhibition of E255V. Concentrations of PD166326 that resulted in a complete growth inhibition and suppression of Bcr-Abl tyrosine phosphorylation, at the same time induced apoptosis of Ba/F3 cells transformed with either of the P loop mutant forms of BCR-ABL (see FIG. 25).

Our results for PD180970, one of the pyrido-pyrimidines tested here, are in line with a recent report showing activity of PD180970 in wild-type and several mutant forms of Bcr-Abl that were examined here as well (La Rosee, P., Corbin, A. S., Stoffregen, E. P., Deininger, M. W., and Druker, B. J. Activity of the Bcr-Abl kinase inhibitor PD180970 against clinically relevant Bcr-Abl isoforms that cause resistance to imatinib mesylate (Gleevec, ST1571). Cancer Res., 62: 7149-7153, 2002; incorporated herein by reference). Relative activity of PD180970 for growth inhibition of Ba/F3 cells expressing wild-type Bcr-Abl and Bcr-Abl H396P, E255K, Y253H, and T3151 is in full compliance with our results with cellular IC₅₀ values that were similar. However, compared to the most active compounds examined here, PD180970 required 5-fold higher concentrations for equivalent suppression of growth and inhibition of Bcr-Abl autophosphorylation, and displayed a narrow range of concentration where specific inhibition of a Bcr-Abl dependent phenotype occured (compare FIGS. 19 and 21; see FIGS. 22 and 24). Using PD180970, cellular IC95 values for Y253H (770 nM) and E255V (935 nM) are in the range or above the concentration where unspecific growth inhibition of parental Ba/F3 cells occurs (IC₅₀ 830 nM).

We conclude, that pyrido-pyrimidine-type small molecule ATP-competitors have two important advantages over imatinib: first, all pyrido-pyrimidines tested here are more potent inhibitors of wild-type Bcr-Abl than imatinib. This could help in completely eliminating the Ph+disease clone as fast as possible and hinder clones with Bcr-Abl amplification to maintain a baseline level of signaling that is sufficient for cell survival in the presence of imatinib. Second, pyrido-pyrimidines differ in their mode of binding to the kinase domain of Abl and therefore are active where imatinib fails. They are capable of suppressing some of the most frequently detected mutations of Bcr-Abl that cause resistance towards imatinib and may therefore be used not only to treat imatinib-resistant disease, but as well to prevent resistant disease clones to emerge. These properties of pyrido-pyrimidines advise therapeutic application in clinical trials. Therefore, ongoing and future studies will determine the pharmacokinetic properties of pyrido-pyrimidines.

### Example 5

### Pharmacokinetic Studies of PD166326, SKI-DV2-281, and SKI-DV2-43

Solvents. PD166326 was dissolved/suspended in a solution of 25% Cremophor +9% ethanol +12% DMSO in saline. SKI-DV281 was dissolved/suspended in a solution of 20% Cremophor +20% ethanol in saline. SKI-DV43 was dissolved/suspended in a solution of 25% Cremophor +9% ethanol +21% DMSO in saline.

Animal Experiments. For each compound, nineteen B6D2F1 mice were used. One mouse was used as control and the rest was divided into two groups with 9 mice per group. One milligram (0.059 μl at 17 mg/ml) of compound was administered to the mice through oral gavage in one group and intravenously in the other group. At each time point (total of 9 time points) after the drug administration, one mouse from each group was sacrificed and approximately 400 μl of blood was collected into heparinized tubes through cardiac puncture. The plasma samples were analyzed as described below.

Analytical. The analysis of drug level in plasma is performed with an automated LC-MS system, which consists of a Prospekt-2 automated sample processor, interfaced with an Agilent 1100 LC-MS. The Prospekt-2 consists of two parts, high pressure eluter (HPE) and automated cartridge exchanger (ACE). The Agilent unit consists of a binary pump, an autosampler, the instrument will perform extraction and analysis automatically. The ACE picks up a new C, 18 solid phase exchange (SPE) cartridge and places it into the clamp. The HPE activates the cartridge by passing acetonitrile and water through the cartridge. The autosampler picks up a predetermined amount of the plasma and the HPE flushes the plasma onto the SPE cartridge, washes it with water, then the sample is analyzed. When the cycle is completed, the ACE will fetch a new cartridge and cycle is repeated. The HPLC mobile phase for the analysis of PD166326 and SKI-DV2-43 consists of 55% acetonitrile and 45% 0.1% formic acid, for the analysis of SKI-DV2-281 is 45% acetonitrile and 55% 20 mM NH₄COOH. The flow rate is 0.4 ml per minute and the eluent is monitored with APCI positive SIM at 427.

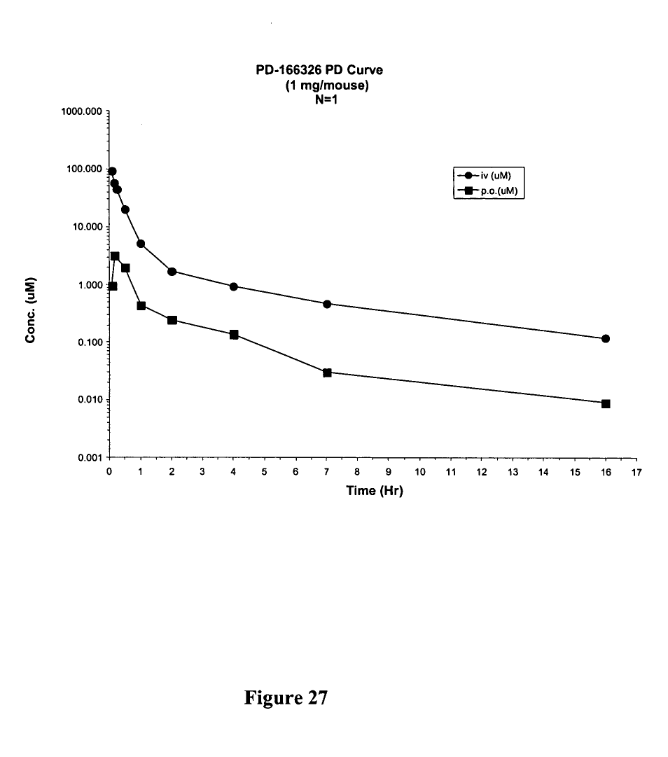

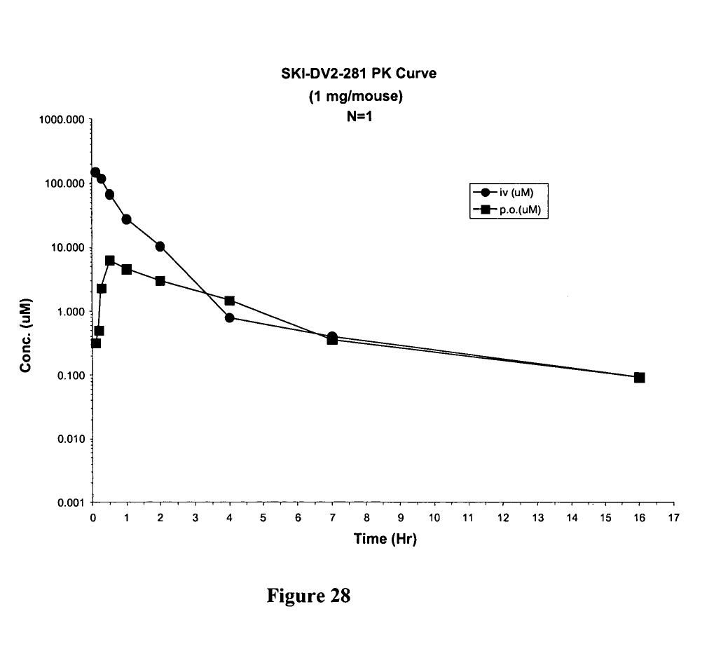

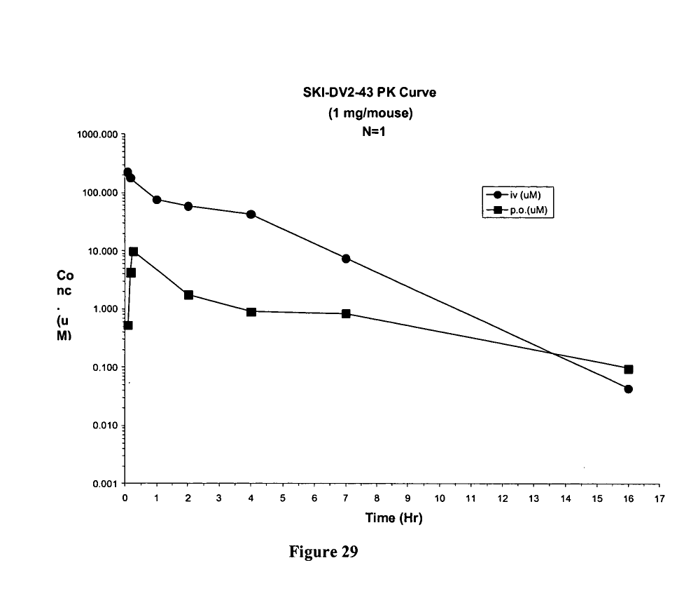

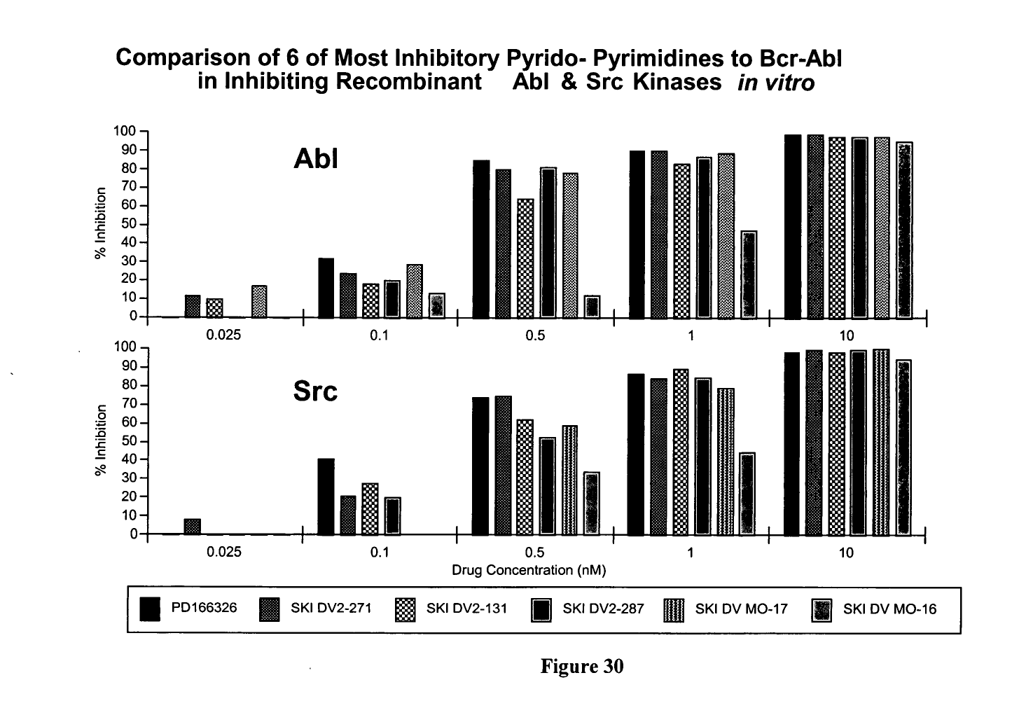

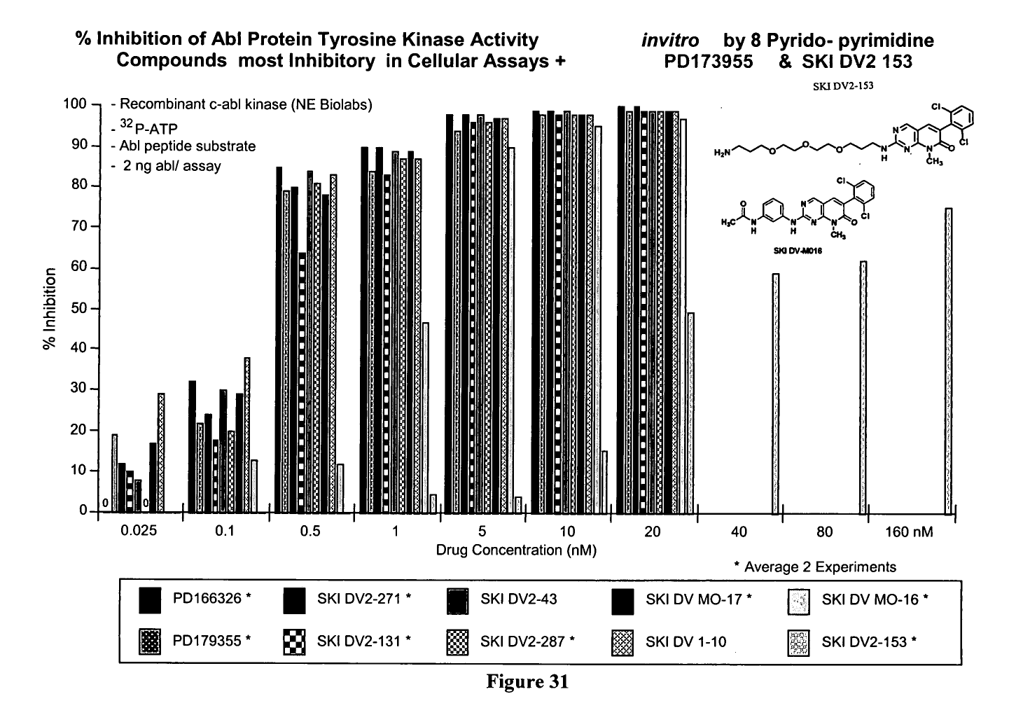

Data analysis and result. The pharmacokinetic curve for each compound was plotted intravenous versus oral (iv vs. po) (see FIGS. 27-29). Data analysis was performed with WinNonlin v.4 (Pharsight) using non-compartmental model. The result is summarized in the table. In comparison to PD166326, SKI-DV2-281 and SKI-DV2-43 show better pharmacokinetic profiles. Both SKI-DV2-281 and SKI-DV2-43 exhibit significantly increased Cₘₐₓ and AUC in either iv group or po group. SKI-DV2-281 has shown a better oral bioavailability. The MRT for SKI-DV2-281 po, SKI-DV2-43 po and iv are longer.

Comparison of Preliminary Pharmacokineticts of Pyrido-pyrimidine
Analogues in B6D2F1 Mice

| | Cmax | | AUG | | MRT | | Oral |
|---|---|---|---|---|---|---|---|
| | (uM) | | (uM × Min) | | (Min) | | Bioavail- |
| Compound | IV | PO | IV | PO | IV | PO | ability |
| PD166326 | 90 | 3.2 | 2395 | 158 | 103 | 126 | 6.6% |
| SKI-DV2-281 | 151 | 6.3 | 6381 | 1047 | 65 | 196 | 16.4% |
| SKI-DV2-43 | 224 | 10 | 20319 | 826 | 174 | 294 | 4% |

*The analysis of plasma compound level was performed with an automated LC-MS system. The data was analyzed by Winnonlin Noncompartmental Analysis Program (Pharsight).

1. A compound of the formula: 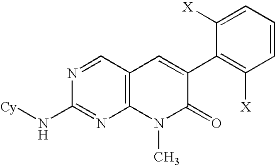
wherein each X is independently hydrogen, fluorine, chlorine, bromine, or iodine; and
Cy is a substituted or unsubstituted cylic aliphatic, cyclic heterocyclic, aryl, heteroaryl, alkylaryl, arylalkyl, alkylheteroaryl, or heteroarylalkyl moiety; and pharmaceutically acceptable derivatives thereof.

2. The compound of claim 1 of formula: 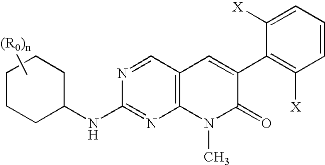
wherein each X independently is hydrogen, fluorine, chlorine, bromine, or iodine;
n is 1, 2, 3, 4, or 5; and
each R₀ is independently hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof.

3. The compound of claim 1, wherein X is chlorine.

4. The compound of claim 1, wherein n is 1.

5. The compound of claim 1 selected from the group consisting of the formulae:

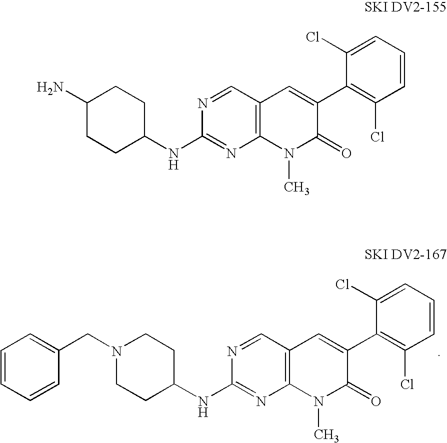

6. The compound of claim 1 of formula: 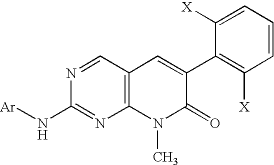
wherein each X is independently hydrogen, fluorine, chlorine, bromine, or iodine; and
Ar is a substituted or unsubstituted aryl, heteroaryl, alkylaryl, or alkylheteroaryl moiety; and pharmaceutically acceptable derivatives thereof.

7. The compound of claim 6, wherein X is chlorine.

8. The compound of claim 6, wherein Ar is a substituted or unsubstituted carbocyclic aromatic moiety.

9. The compound of claim 6, wherein Ar is a substituted or unsubstituted heterocyclic aromatic moiety.

10. The compound of claim 6, wherein Ar is a substituted or unsubstituted five- or six-membered aromatic moiety.

11. The compound of claim 6, wherein Ar is phenyl or pyridinyl.

12. The compound of claim 6 of formula: 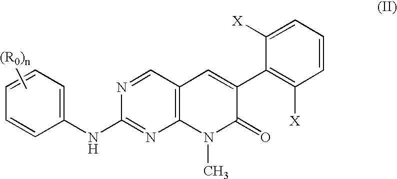
wherein each X independently is hydrogen, fluorine, chlorine, bromine, or iodine;
n is 1, 2, 3, 4, or 5; and
each R₀ is independently hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof.

13. The compound of claim 12, wherein X is chlorine.

14. The compound of claim 12, wherein n is 1, 2, or 3.

15. The compound of claim 12, wherein n is 1.

16. The compound of claim 12, wherein each R₀ is independently amino, hydroxy, hydroxymethyl, acetamido, iodo, fluoro, bromo, iodo, ethyl, propyl, cyclopropyl, butyl, cyclopbutyl, pentyl, hexyl, cyclohexyl, (6-biotinamido)hexamido, (2,3-dihydroxypropoxy)methyl, (2,3-dihydropropyl)amino, (acrylamido)phenylamido, or 4-methylpiperazinylcarboxy.

17. The compound of claim 6 of the formula: 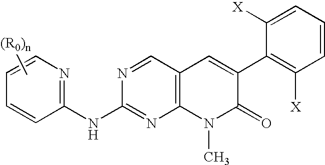
wherein each X is independently hydrogen, fluorine, chlorine, bromine, or iodine;
n is 1, 2, 3, 4, or 5; and
each R₀ is independently hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof.

18. The compound of claim 6 of formula: 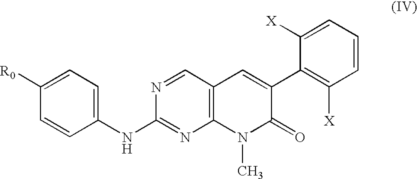
wherein each X is independently hydrogen, fluorine, chlorine, bromine, or iodine; and
R₀ is hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof.

19. The compound of claim 6 selected from the group consisting of the formulae:

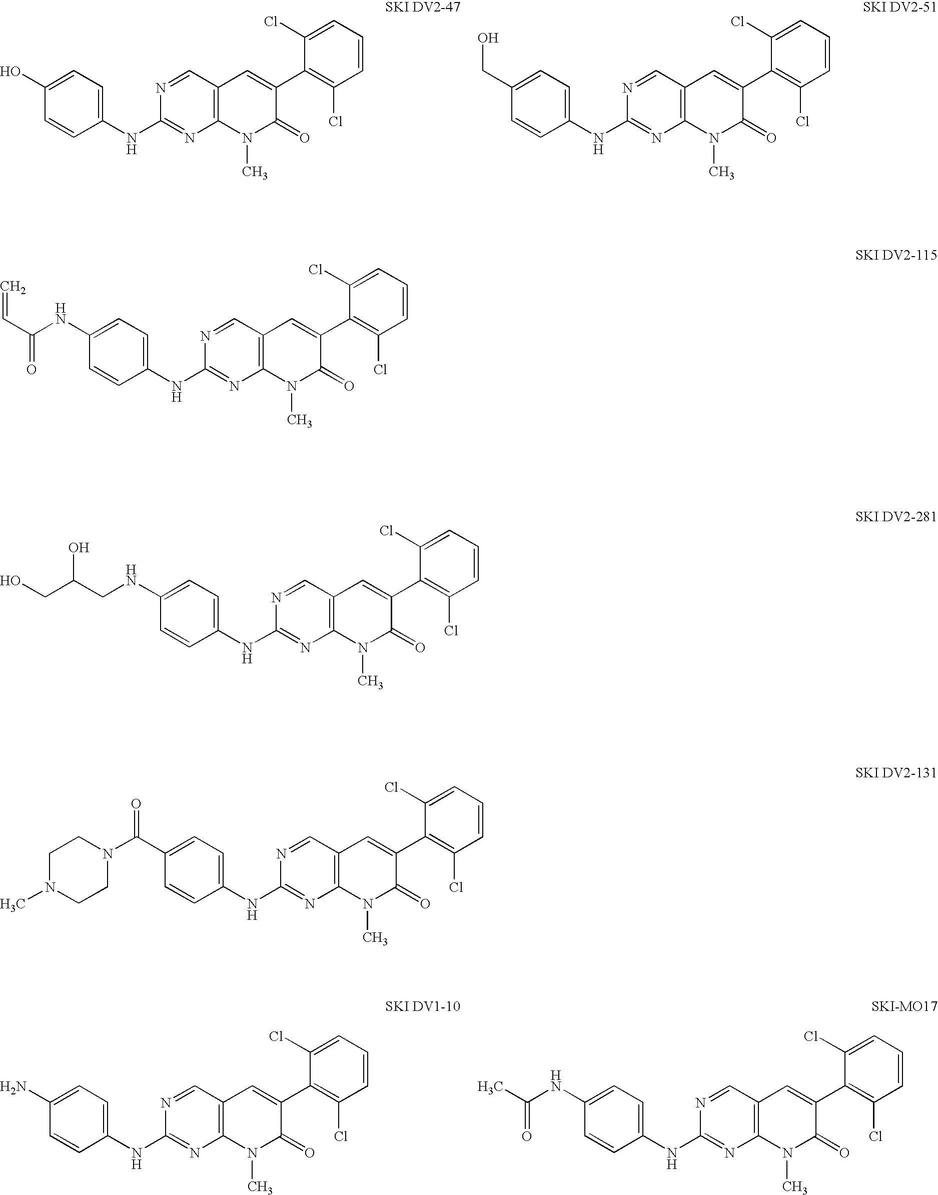

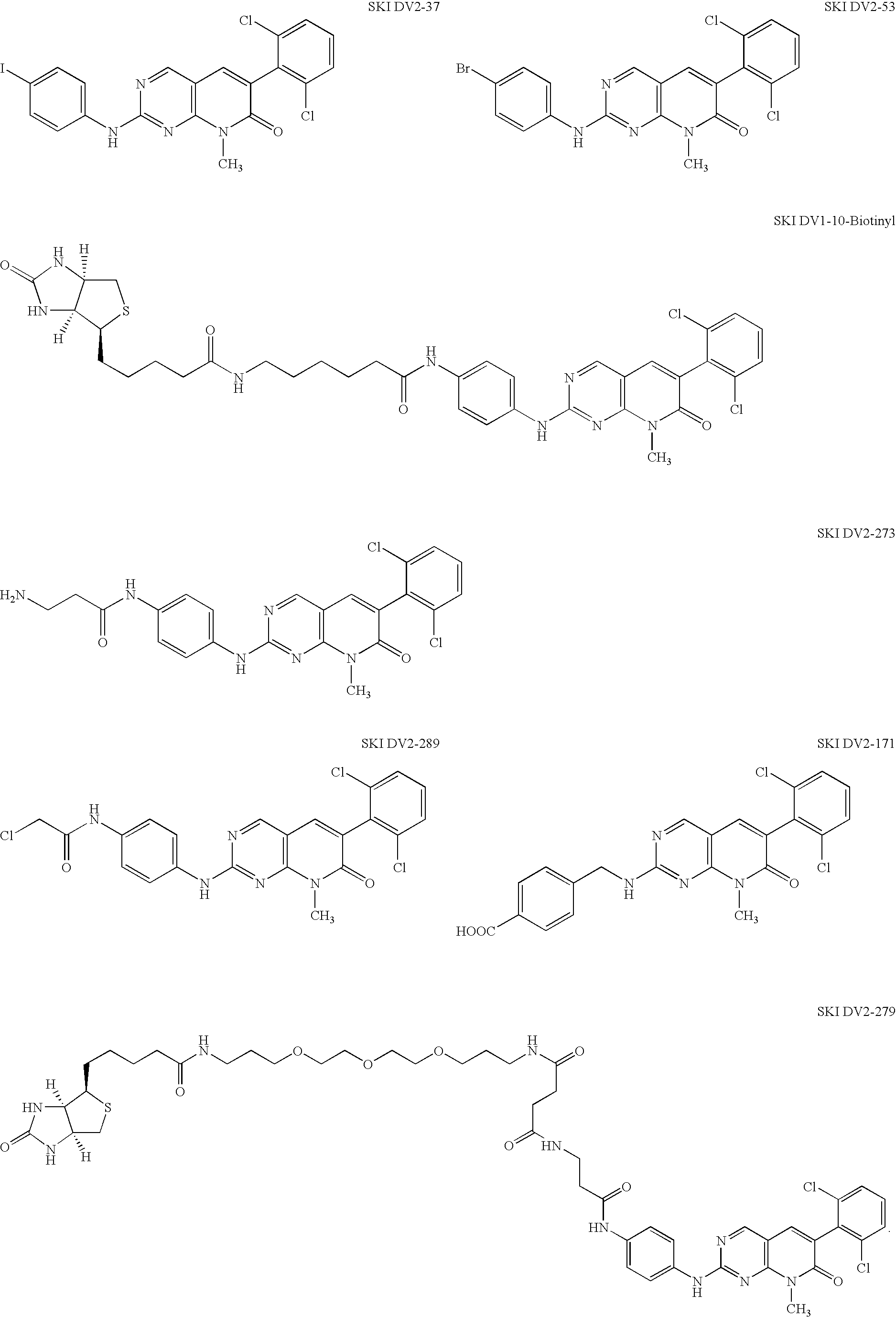

20. The compound of claim 6 of formula:

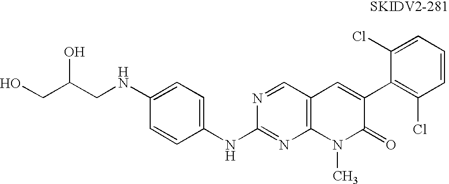

21. The compound of claim 6 of formula: 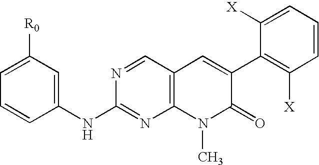
wherein each X is independently hydrogen, fluorine, chlorine, bromine, or iodine; and
R₀ is hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof.

22. The compound of claim 6 selected from the group consisting of formulae:

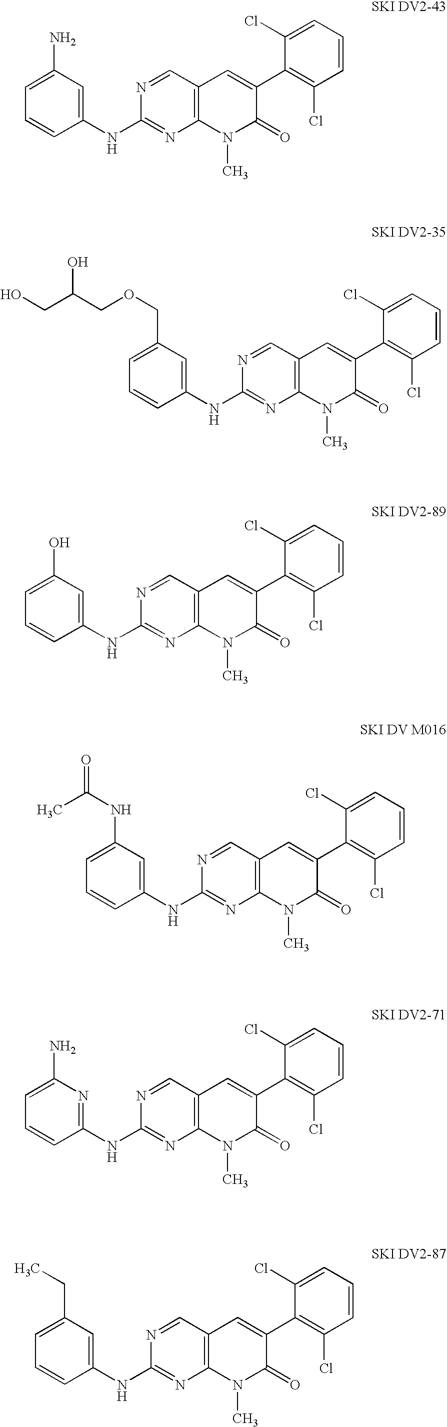

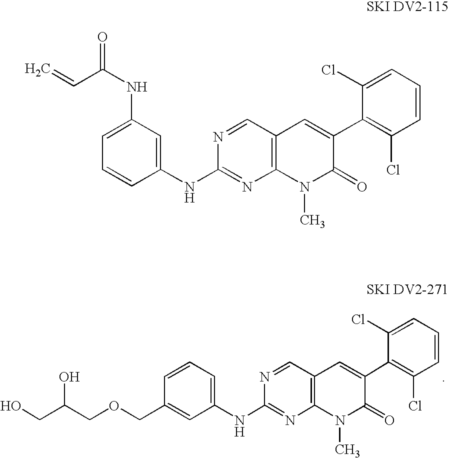

23. The compound of claim 6 of formula: 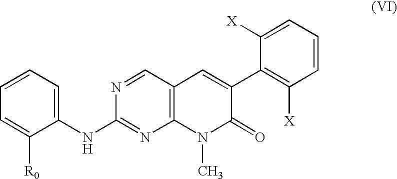
wherein each X is independently hydrogen, fluorine, chlorine, bromine, or iodine; and
R₀ is hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof.

24. The compound of claim 6 of formula: 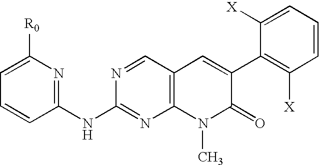
wherein each X is independently hydrogen, fluorine, chlorine, bromine, or iodine; and
R₀ is hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino; and pharmaceutically acceptable derivatives thereof.

25. A compound of formula: 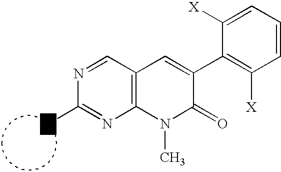
wherein each X is independently hydrogen, fluorine, chlorine, bromine, or iodine; and 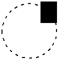
is a substituted or unsubstituted heterocyclic moiety, wherein 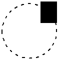
is aromatic or nonaromatic; and pharmaceutically acceptable derivatives thereof.

26. The compound of claim 25, wherein 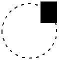
is a pyrrolidine, piperidine, aziridine, azetidine, pyridine, pyrrole, oxazole, thiazole, indole, purine, carbazole, imidazole, isoxazole, pyrazole, or isothiazole moiety.

27. The compound of claim 25 of formula:

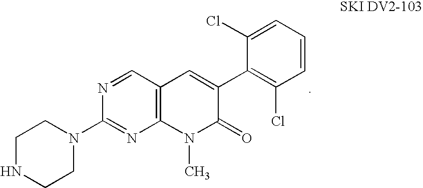

28. A compound of the formula: 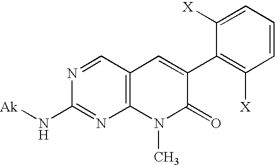
wherein each X is independently hydrogen, fluorine, chlorine, bromine, or iodine; and
Ak is a substituted or unsubstituted aliphatic or heterocyclicl moiety; and
pharmaceutically acceptable derivatives thereof.

29. The compound of claim 28 wherein X is chlorine.

30. The compound of claim 28 of the formula:

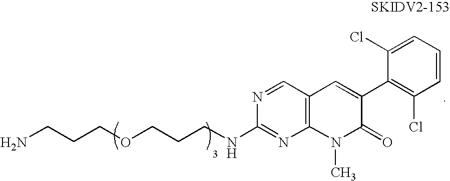

31. A method of preparing a compound of formula (II): 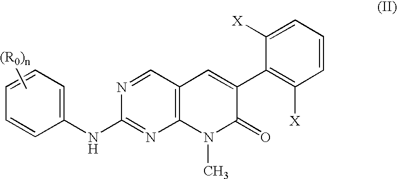
wherein each X independently is hydrogen, fluorine, chlorine, bromine, or iodine;
n is 1, 2, 3, 4, or 5; and
each R₀ is independently hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino;
the method comprising steps of:
(a) reacting 4-methylamino-2-methylsulfanyl-pyrimidine-5-carbaldehdye: 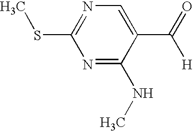
with a 2,6-dihalophenylacetonitrile, 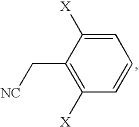
under basic conditions to form a pyrido[2,3-d]pyrimidin-7-ylideneamine of formula: 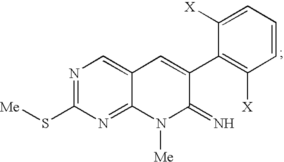
(b) treating the resulting pyrido[2,3-d]pyrimidin-7-ylideneamine with acetic anhydride and acid to form the corresponding pyrido[2,3-d]pyrimidin-7-one: 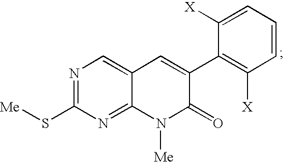
(c) oxidizing the pyrido[2,3-d]pyrimidin-7-one to form a sulfone of formula: 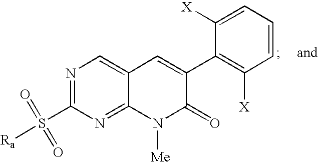
and
(d) coupling the sulfone with an aniline of formula: 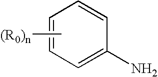
under suitable conditions to form the compound of formula (II).

32. A method of preparing a compound of formula (II): 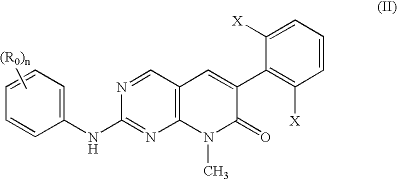
wherein each X independently is hydrogen, fluorine, chlorine, bromine, or iodine;
n is 1, 2, 3, 4, or 5; and
each R₀ is independently hydrogen; halogen; amino; protected amino; amino substituted with one or two alkyl or aryl moieties; alkoxy, carboxy, carboxaldehyde; acyl; linear or branched alkyl or cyclic acetal; substituted or unsubstituted, linear or branched, cyclic or acyclic aliphatic, heteroaliphatic, aryl, heteroaryl, arylalkyl, or heteroarylalkyl moiety, optionally substituted by one or more of hydroxy, protected hydroxy, alkoxy, carboxy, carboxaldehyde, linear or branched alkyl or cyclic acetal, halogen, amino, protected amino, amino substituted with one or two alkyl or aryl moieties, N-hydroximino, or N-alkoxyimino;
the method comprising steps of:
(a) reacting a sulfone having the structure: 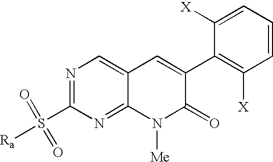
wherein Rₐ is a C₁-C₆ alkyl moiety;
with an aniline of structure: 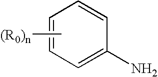
under suitable conditions to form the compound of formula (II).

33. A pharmaceutical composition comprising a compound of claim 1 and a pharmaceutically acceptable excipient.

34. A method of treating cancer comprising:
administering a therapeutically effective amount of a compound of claim 1 to a subject in need thereof.

35. The method of claim 34, wherein the therapeutically effective amount of the compound is an amount sufficient to deliver about 0.01 mg to about 75 mg compound per kg body weight.

36. The method of claim 34, wherein the cancer is drug resistant.

37. The method of claim 36, wherein the cancer is resistant to at least one other tyrosine kinase inhibitor.

38. The method of claim 36, wherein the cancer is resistant to Gleevec® (imatinib mesylate).

39. The method of claim 34 further comprising administering a therapeutically effective amount of an anti-neoplastic agent in addition to a compound of claim 1.

40. The method of claim 39 wherein the anti-neoplastic agent is a chemically unrelated tyrosine kinase inhibitor.
